(19) United States
(12) Patent Application Publication
(10) Pub. No.: US 2014/0259220 A1
(43) Pub. Date: Sep. 11, 2014

(54) MANIPULATING PHB GENES FOR PLANT BIOMASS ACCUMULATION AND YIELD

(71) Applicant: The Texas A&M University System, College Station, TX (US)

(72) Inventor: Shuhua Yuan, College Station, TX (US)

(73) Assignee: The Texas A&M University System, College Station, TX (US)

(21) Appl. No.: 14/198,543

(22) Filed: Mar. 5, 2014

Related U.S. Application Data
(60) Provisional application No. 61/773,011, filed on Mar. 5, 2013.

(51) Int. Cl. C12N 15/82

(52) U.S. Cl. CPC C12N 15/8261; C12N 15/8279
USPC 800/279; 435/417; 435/415; 435/414; 435/412; 435/419; 435/411; 435/418; 800/290; 800/298

(57) ABSTRACT

The present invention provides methods for increasing plant biomass and plant seed yield through overexpression of a PHB gene. Also provided are plants with increased biomass and seed yield comprising overexpression of a PHB gene produced by such methods. Plants described herein may be used, for example, for improved production of biofuels.

### CROSS REFERENCE TO RELATED APPLICATIONS

This application claims the benefit of U.S. Provisional Application No. 61/773,011, filed Mar. 5, 2013, herein incorporated by reference in its entirety.

### FIELD OF THE INVENTION

The present invention relates generally to the field of molecular biology. More specifically, the invention relates to plant genes involved in plant morphology and methods of use thereof.

### INCORPORATION OF SEQUENCE LISTING

The sequence listing that is contained in the file named “TAMC023US_ST25.txt,” which is 592 kilobytes as measured in Microsoft Windows operating system and was created on Mar. 5, 2014, is filed electronically herewith and incorporated herein by reference.

### BACKGROUND OF THE INVENTION

Genetic modification of plants has, in combination with conventional breeding programs, led to significant increases in agricultural yield over the last decades. Genetically modified plants may be selected for a single agronomic trait, for example by expression of a single enzyme coding sequence (e.g., enzymes that provide herbicide resistance). Genetic manipulation of genes involved in plant growth and yield may enable increased production of valuable commercial crops, resulting in benefits in agriculture and development of alternate energy sources such as biofuels. Accordingly, methods capable of increasing plant biomass and seed yield through gene regulation are described.

### SUMMARY OF THE INVENTION

In one aspect, the present invention provides a method of altering plant biomass comprising increasing expression of a PHB8 gene in a plant, wherein the biomass of the plant is increased when compared to a plant that lacks the increased expression. In another aspect, the invention provides a method of altering plant biomass comprising increasing expression of an ATP synthase gene in a plant, wherein the biomass of the plant is increased when compared to a plant that lacks the increased expression. In an embodiment, a plant with increased biomass comprises over-expression or increased expression of a gene that encodes a PHB8 or an ATP synthase (ATPase). In another embodiment, a plant having increased biomass in accordance with the invention may comprise over-expression or up-regulated expression of a PHB8 gene in combination with over-expression or up-regulated expression of an ATP synthase or ATPase. An ATP synthase in accordance with the invention may comprise multiple subunits or protein chains, for example, At5g08670, At5g08680, and At5g08690, set forth as SEQ ID NOs:11-13, respectively.

In certain embodiments, the plant may be a dicotyledonous plant such as Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. or a monocotyledonous plant such as maize, wheat, rice, sorghum (Sorghum bicolor), oats, barley, sugar cane, African oil palm (Elaeis guineensis), or switchgrass. In another embodiment, the plant has altered morphology when compared to a plant that lacks the increased expression, such as altered increased plant biomass or increased seed yield.

In another aspect, the invention provides a plant comprising increased expression of a PHB8 gene, wherein the biomass of the plant is increased when compared to a plant that lacks the increased expression. In embodiments, the invention provides a seed that produces such a plant, a seed produced by such a plant, or a DNA-containing plant part of such a plant. In another embodiment, the plant part is a protoplast, cell, meristem, root, leaf, node, pistil, anther, flower, seed, embryo, stalk or petiole.

In another aspect, the invention provides a method of altering seed yield in a plant, the method comprising increasing expression of a PHB8 gene in the plant, wherein the seed yield of the plant is increased when compared to a plant that lacks the increased expression. In certain embodiments, the plant may be a dicotyledonous plant such as Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. or a monocotyledonous plant such as maize, wheat, rice, sorghum (Sorghum bicolor), oats, barley, sugar cane, African oil palm (Elaeis guineensis), or switchgrass. In another embodiment, the plant has altered morphology when compared to a plant that lacks the increased expression, such as altered increased plant biomass or increased seed yield.

In another aspect, the invention provides a plant comprising increased expression of a PHB8 gene, wherein the seed yield of the plant is increased when compared to a plant that lacks the increased expression. In embodiments, the invention provides a seed that produces such a plant, a seed produced by such a plant, or a DNA-containing plant part of such a plant. In another embodiment, the plant part is a protoplast, cell, meristem, root, leaf, node, pistil, anther, flower, seed, embryo, stalk or petiole.

In another aspect, the invention provides a method of producing increased plant biomass, the method comprising: (a) obtaining a plant comprising increasing expression of a PHB8 gene, wherein the biomass of the plant is increased when compared to a plant that lacks the increased expression; (b) growing said plant under plant growth conditions to produce plant tissue from the plant; and (c) preparing biomass from said plant tissue. In certain embodiments, preparing biomass comprises harvesting said plant tissue or using the biomass for biofuel.

In another aspect, the invention provides a method for protecting a plant from pest infestation comprising: (i) increasing expression of a PHB8 gene in a plant; and (ii) providing in the diet of a plant pest an insecticidally effective amount of cells or tissue of the plant or part thereof; wherein the insecticidal properties of the plant are increased when compared to a plant that lacks the increased expression. In certain embodiments, the plant may be a dicotyledonous plant such as Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. or a monocotyledonous plant such as maize, wheat, rice, sorghum (Sorghum bicolor), oats, barley, sugar cane, African oil palm (Elaeis guineensis), or switchgrass. In another embodiment, the plant has altered morphology when compared to a plant that lacks the increased expression, such as altered increased plant biomass or increased seed yield.

In another aspect, the invention provides a polynucleotide molecule comprising a PHB8 sequence selected from the group consisting of: (a) a polynucleotide sequence comprising the nucleic acid sequence of SEQ ID NOs:1-10; (b) a polynucleotide sequence having at least about 90% sequence identity to the sequence of SEQ ID NOs:1-10, or (c) a polynucleotide sequence encoding a polypeptide sequence set forth as SEQ ID NOs:97-195. In an embodiment, the sequence is an Arabidopsis thaliana sequence or a homolog thereof. In another embodiment, the invention provides a polypeptide encoded by such a polynucleotide molecule. In another embodiment, the invention provides a recombinant nucleic acid construct comprising such a polynucleotide molecule operably linked to a heterologous promoter. In another embodiment, the invention provides such a recombinant nucleic acid construct, wherein the promoter is functional in plant cells.

In another aspect, the invention provides a polypeptide comprising a sequence selected from the group consisting of: (a) a polypeptide sequence comprising a PHB8 protein selected from the group consisting of SEQ ID NOs:97-195; (b) a polypeptide sequence having at least about 90% sequence identity to the sequence of SEQ ID NOs:97-195. In one embodiment, the sequence is an Arabidopsis thaliana sequence or a homolog thereof.

In another aspect, the invention provides a polynucleotide molecule comprising an ATP synthase sequence selected from the group consisting of: (a) a polynucleotide sequence comprising the nucleic acid sequence of SEQ ID NOs:11-13 and 59-96; (b) a polynucleotide sequence having at least about 90% sequence identity to the sequence of SEQ ID NOs:11-13 and 59-96; or (c) a polynucleotide sequence encoding a polypeptide sequence set forth as SEQ ID NOs:14-58. In one aspect, the sequence is an Arabidopsis thaliana sequence or a homolog thereof. In an embodiment, the invention provides a polypeptide encoded by such a polynucleotide molecule. In another embodiment, the invention provides a recombinant nucleic acid construct comprising such a polynucleotide molecule linked to a heterologous promoter. In another embodiment, the invention provides a recombinant nucleic acid construct, wherein the promoter is functional in plant cells.

In another aspect, the invention provides a polypeptide comprising a sequence selected from the group consisting of: (a) a polypeptide sequence comprising an ATP synthase protein selected from the group consisting of SEQ ID NOs:14-58; (b) a polypeptide sequence having at least about 90% sequence identity to the sequence of SEQ ID NOs:14-58. In an embodiment, the invention provides a polypeptide sequence, wherein the sequence is an Arabidopsis thaliana sequence or a homolog thereof.

In one aspect, the present invention provides a method of altering plant biomass comprising increasing expression of an ATP synthase gene in a plant, wherein the biomass of the plant is increased when compared to a plant that lacks the increased expression.

In certain embodiments, the plant may be a dicotyledonous plant such as Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. or a monocotyledonous plant such as maize, wheat, rice, sorghum (Sorghum bicolor), oats, barley, sugar cane, African oil palm (Elaeis guineensis), or switchgrass. In another embodiment, the plant has altered morphology when compared to a plant that lacks the increased expression, such as altered increased plant biomass or increased seed yield.

In another aspect, the invention provides a plant comprising increased expression of an ATP synthase gene, wherein the biomass of the plant is increased when compared to a plant that lacks the increased expression. In embodiments, the invention provides a seed that produces such a plant, a seed produced by such a plant, or a DNA-containing plant part of such a plant. In another embodiment, the plant part is a protoplast, cell, meristem, root, leaf, node, pistil, anther, flower, seed, embryo, stalk or petiole.

In another aspect, the invention provides a method of altering seed yield in a plant, the method comprising increasing expression of an ATP synthase gene in the plant, wherein the seed yield of the plant is increased when compared to a plant that lacks the increased expression. In certain embodiments, the plant may be a dicotyledonous plant such as Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. or a monocotyledonous plant such as maize, wheat, rice, sorghum (Sorghum bicolor), oats, barley, sugar cane, African oil palm (Elaeis guineensis), or switchgrass. In another embodiment, the plant has altered morphology when compared to a plant that lacks the increased expression, such as altered increased plant biomass or increased seed yield.

In another aspect, the invention provides a plant comprising increased expression of an ATP synthase gene, wherein the seed yield of the plant is increased when compared to a plant that lacks the increased expression. In embodiments, the invention provides a seed that produces such a plant, a seed produced by such a plant, or a DNA-containing plant part of such a plant. In another embodiment, the plant part is a protoplast, cell, meristem, root, leaf, node, pistil, anther, flower, seed, embryo, stalk or petiole.

In another aspect, the invention provides a method of producing increased plant biomass, the method comprising: (a) obtaining a plant comprising increasing expression of an ATP synthase gene, wherein the biomass of the plant is increased when compared to a plant that lacks the increased expression; (b) growing said plant under plant growth conditions to produce plant tissue from the plant; and (c) preparing biomass from said plant tissue. In certain embodiments, preparing biomass comprises harvesting said plant tissue or using the biomass for biofuel.

In another aspect, the invention provides a method for protecting a plant from pest infestation comprising: (i) increasing expression of an ATP synthase gene in a plant; and (ii) providing in the diet of a plant pest an insecticidally effective amount of cells or tissue of the plant or part thereof; wherein the insecticidal properties of the plant are increased when compared to a plant that lacks the increased expression. In certain embodiments, the plant may be a dicotyledonous plant such as Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. or a monocotyledonous plant such as maize, wheat, rice, sorghum (Sorghum bicolor), oats, barley, sugar cane, African oil palm (Elaeis guineensis), or switchgrass. In another embodiment, the plant has altered morphology when compared to a plant that lacks the increased expression, such as altered increased plant biomass or increased seed yield.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1: (A) Shows the phenotypes of Arabidopsis thaliana PHB knockout line phb8-1 and PHB8 over-expression line OE-11 compared to the wild-type (WT) phenotype. OE-11 comprises the PHB8 gene under the control of the CaMV 35S promoter. (B) Shows over-expression of PHB8 in Arabidopsis resulted in early bolting compared to WT plants.

FIG. 2: Shows stem size and seed yield of knockout line phb8-1, WT, and over-expression lines (OE-11-7). Asterisks indicate significant differences (p<0.05) between WT and OE11-7, or WT and phb8-1. Overexpression of the PHB8 gene in Arabidopsis resulted in an increase in stalk height of 20% (A), an increase in stem diameter of 20% (B), and an increase in seed number of 50% (C and D).

FIG. 3: Shows localization of PHB8 protein in the mitochondria. PHB8 fused with GFP (green fluorescent protein) accumulated in mitochrondria of protoplasts from Arabidopsis leaf. Left: Transient co-expression of PHB8-GFP; Middle: Mitochondrial marker fused with red fluorescent protein (RFP); and Right: Overlay of left and middle images.

FIG. 4: Shows ATP levels between WT, over-expression lines OE4-14 and OE11-7, and phb8-1 Arabidopsis lines. Arabidopsis phb8 over-expressing lines accumulated ATP to higher levels than WT and mutant lines. Asterisks indicate significant differences (p<0.05) between WT and OE4-14 and OE11-7. FW refers to fresh weight.

FIG. 5: Shows the phenotype of Arabidopsis lines over-expressing ATPase. (A) Shows fast growth rate (upper) and early flowering (lower) of Arabidopsis lines over-expressing ATPase compared to WT. (B) Shows the higher stem height of Arabidopsis lines over-expressing ATPase compared to WT.

FIG. 6: (A) Shows leaf area damage to WT (upper) and PHB8 over-expression (lower) rice lines made by the fall armyworm (Spodoptera frugiperda). (B) Shows qRT-PCR results demonstrating relative expression levels for WT and PHB8 over-expression transgenic rice lines.

### BRIEF DESCRIPTION OF THE SEQUENCE LISTING

SEQ ID NO:1—Sequence of the full-length cDNA of the PHB8 gene (AT3g01290) in Arabidopsis thaliana.

SEQ ID NO:2—Sequence of the full-length cDNA of the PHB8 gene (LOC_Os10g32700) in Oryza sativa Japonica.

SEQ ID NO:3—Sequence of the full-length cDNA of the PHB8 gene (ZM_BFb0049N19) in Zea mays.

SEQ ID NO:4—Sequence of the full-length cDNA of the PHB8 gene (SORBI_{—}02g022890) in Sorghum bicolor.

SEQ ID NO:5—Sequence of the full-length cDNA of the PHB8 gene (LOC100804982) in Glycine max.

SEQ ID NO:6—Sequence of the partial coding sequence of the PHB8 gene (AEZ00872.1) in Elaeis guineensis.

SEQ ID NO:7—Sequence of the partial coding sequence of the PHB8 gene (JN596997.1) in Arachis hypogaea.

SEQ ID NO:8—Sequence of the full-length cDNA of the PHB8 gene (LOC100245034) in Vitis vinifera.

SEQ ID NO:9—Sequence of the full-length cDNA of the PHB8 gene (RCOM_{—}0797990) in Ricinus communis.

SEQ ID NO:10—Sequence of the full-length cDNA of the PHB8 gene (MTR_{—}4g107760) in Medicago truncatula.

SEQ ID NO:11—Sequence of a mitochondrial ATP synthase beta-subunit (AT5G08670).

SEQ ID NO:12—Sequence of a mitochondrial ATP synthase beta-subunit (AT5G08680).

SEQ ID NO:13—Sequence of a mitochondrial ATP synthase beta-subunit (AT5G08690).

SEQ ID NOs: 14-58—Protein sequences of homologs of ATP synthase (ATPase).

SEQ ID NO:59-96—DNA coding sequences of homologs of ATP synthase (ATPase).

SEQ ID NO:97-195—Protein sequences of homologs of PHB8.

### DETAILED DESCRIPTION OF THE INVENTION

The present invention provides a method of altering biomass in a plant by increasing expression of the PHB8 gene. Plants of the present invention that overexpress the PHB8 gene demonstrate increased biomass, such as increased plant stalk height, increase plant biomass, increased plant stem diameter, increased crop yield, increased number of seeds, increased number of siliques, increased number of seeds per silique, and increased levels of ATP when compared to a plant that lacks the increased expression.

While the PHB genes in mammals are known in the art to be associated with cancer and aging, and overexpression of PHB genes in mammals has been reported to result in decreased cell division, overexpression of PHB in plants in accordance with the present invention results in increased plant biomass, such as size and/or number of seeds produced, and thus may also function in other areas of plant development and growth.

In one embodiment, a plant in accordance with the invention having increased biomass may comprise over-expression of a PHB gene, such as PHB8 (SEQ ID NOs:1-10). In another embodiment, a plant with increased biomass may comprise over-expression of a gene that encodes a mitochondrial protein. Mitochondrial proteins that may be useful for the present invention may include, but are not limited to, an ATP synthase (ATPase) or an NAD(P)H dehydrogenase. In another embodiment, a plant having increased biomass in accordance with the invention may comprise over-expression or up-regulated expression of a PHB8 gene in combination with over-expression or up-regulated expression of an ATP synthase or ATPase. An ATP synthase in accordance with the invention may be an F1/F0-type ATP synthase, which produces ATP via a proton gradient. An ATP synthase in accordance with the invention may comprise multiple subunits or protein chains, for example, At5g08670, At5g08680, and At5g08690, set forth as SEQ ID NOs:11-13, respectively, or another ATP synthase (ATPase) sequence set forth as SEQ ID NOs:59-96. In accordance with the invention, nucleic acid and/or protein sequences may share sequence identity at the nucleic acid or amino acid level. For example, such sequences may share 100%, 99%, 98%, 97%, 96%, 95%, 94%, 93%, 92%, 91%, 90%, 89%, 88%, 87%, 86%, 85%, 84%, 83%, 82%, 81%, 80% sequence identity, or the like.

Without being limited to a particular theory, a plant useful for the present invention may be a dicot, for example Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. In another embodiment, a plant useful for the present invention may be a monocot, for example maize, wheat, rice, sorghum (Sorghum bicolor), oats, barley, sugar cane, African oil palm (Elaeis guineensis), or switchgrass.

### I. NUCLEIC ACIDS, POLYPEPTIDES AND PLANT TRANSFORMATION CONSTRUCTS

Certain embodiments of the current invention concern isolated nucleic acid sequences comprising a PHB coding sequence, set forth as SEQ ID NOs:1-10. Complements to any nucleic acid sequences described herein are also provided.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. Methods to determine “identity” are designed to give the largest match between the sequences tested. Moreover, methods to determine identity are codified in publicly available programs. “Identity” can be readily calculated by known methods including, but not limited to, those described in Lesk, ed., (1988); Smith, ed., (1993); Griffin, and Griffin, eds., (1994); von Heinje, (1987); Gribskov and Devereux, eds., (1991); and Carillo and Lipman, (1988). Computer programs can be used to determine “identity” between two sequences these programs include but are not limited to, GCG (Devereux, 1984); suite of five BLAST programs, three designed for nucleotide sequences queries (BLASTN, BLASTX, and TBLASTX) and two designed for protein sequence queries (BLASTP and TBLASTN) (Coulson, 1994; Birren, et al., 1997). The BLASTX program is publicly available from NCBI and other sources (BLAST Manual, Altschul, S., et al., NCBI NLM NIH, Bethesda, Md. 20894; Altschul, S., et al., 1990). The well known Smith Waterman algorithm can also be used to determine identity.

Parameters for polypeptide sequence comparison include the following: Algorithm: Needleman and Wunsch (1970); Comparison matrix: BLOSUM62 from Hentikoff and Hentikoff, (1992); Gap Penalty: 12; and Gap Length Penalty: 4. A program which can be used with these parameters is publicly available as the “gap” program from Genetics Computer Group, Madison Wis. The above parameters along with no penalty for end gap may serve as default parameters for peptide comparisons.

Parameters for nucleic acid sequence comparison include the following: Algorithm: Needleman and Wunsch (1970); Comparison matrix: matches=+10; mismatches=0; Gap Penalty: 50; and Gap Length Penalty: 3. A program which can be used with these parameters is publicly available as the “gap” program from Genetics Computer Group, Madison Wis. The above parameters may serve as the default parameters for nucleic acid comparisons.

As used herein, “hybridization,” “hybridizes,” or “capable of hybridizing” is understood to mean the forming of a double- or triple-stranded molecule or a molecule with partial double- or triple-stranded nature. Such hybridization may take place under relatively high-stringency conditions, including low salt and/or high temperature conditions, such as provided by a wash in about 0.02 M to about 0.15 M NaCl at temperatures of about 50° C. to about 70° C. for 10 min. In one embodiment of the invention, the conditions are 0.15 M NaCl and 70° C. Stringent conditions tolerate little mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

The nucleic acids provided herein as SEQ ID NOs:1-10 may be from any source, e.g., identified as naturally occurring in a plant, or synthesized, e.g., by mutagenesis of SEQ ID NOs:1-10. In an embodiment, the naturally occurring sequence may be from any plant. In some embodiments, the plant may be a dicotyledonous plant, for example, Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp. In other embodiments, a plant useful for the present invention may be a monocotyledonous plant, for example maize, wheat, rice, sorghum (Sorghum bicolor), oats, barley, sugar cane, African oil palm (Elaeis guineensis), or switchgrass.

Coding sequences may be provided in a recombinant vector operably linked to a heterologous promoter functional in plants, in either sense or antisense orientation. Expression constructs may also be provided comprising these sequences, including antisense oligonucleotides thereof. In other embodiments, plants and plant cells transformed with the sequences may be provided. The construction of vectors which may be employed in conjunction with plant transformation techniques using these or other sequences according to the invention will be known to those of skill of the art in light of the present disclosure (see, for example, Sambrook et al., 1989; Gelvin et al., 1990). The techniques of the current invention are thus not limited to any particular nucleic acid sequences.

The choice of any additional elements used in conjunction with the PHB coding sequences may depend on the purpose of the transformation. One of the major purposes of transformation of crop plants is to add commercially desirable, agronomically important traits to the plant, as described above.

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce genes corresponding to, e.g., an entire biosynthetic pathway, into a plant.

Particularly useful for transformation are expression cassettes which have been isolated from such vectors. DNA segments used for transforming plant cells will generally comprise the cDNA, gene, or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. Preferred components likely to be included with vectors used in the current invention are as follows.

A. Regulatory Elements

Exemplary promoters for expression of a nucleic acid sequence include plant promoters such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), α-tubulin, actin (Wang et al., 1992), cab (Sullivan et al., 1989), PEPCase (Hudspeth and Grula, 1989) or those promoters associated with the R gene complex (Chandler et al., 1989). Tissue-specific promoters such as leaf specific promoters, or tissue selective promoters (e.g., promoters that direct greater expression in leaf primordia than in other tissues), and tissue-specific enhancers (Fromm et al., 1986) are also contemplated to be useful, as are inducible promoters such as ABA- and turgor-inducible promoters. Any suitable promoters known in the art may be used to express PHB coding sequences in a plant. In an embodiment of the invention, the CaMV35S promoter may be used to express PHB coding sequences in a plant.

The DNA sequence between the transcription initiation site and the start of the coding sequence, i.e., the untranslated leader sequence, can also influence gene expression. One may thus wish to employ a particular leader sequence with a transformation construct of the invention. In an embodiment, leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure. In some embodiments, sequences that are derived from genes that are highly expressed in plants may be used for expression of PHB coding sequences.

It is envisioned that PHB coding sequences may be introduced under the control of novel promoters, enhancers, etc., or homologous or tissue-specific or tissue-selective promoters or control elements. Vectors for use in tissue-specific targeting of genes in transgenic plants will typically include tissue-specific or tissue-selective promoters and may also include other tissue-specific or tissue-selective control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters, which have higher activity in roots.

B. Terminators

Transformation constructs prepared in accordance with the invention may include a 3′ end DNA sequence that acts as a signal to terminate transcription and allow for the polyadenylation of the mRNA produced by coding sequences operably linked to a promoter. In one embodiment of the invention, the native terminator of a PHB coding sequence may be used. Alternatively, a heterologous 3′ end may enhance the expression of sense or antisense PHB coding sequences. Examples of terminators that may be used in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II gene from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, Golgi apparatus, and outside of the cellular membrane). By facilitating the transport of the protein into compartments inside and outside the cell, these sequences may increase the accumulation of gene products by protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit or signal peptide will transport the protein to a particular intracellular or extracellular destination, respectively, and will then be post-translationally removed.

D. Marker Genes

By employing a selectable or screenable marker, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

One beneficial use of the sequences provided by the invention may be in the alteration of plant phenotypes by genetic transformation with PHB coding sequences. The PHB coding sequence may be provided with other sequences. Where an expressible coding region that is not necessarily a marker coding region is employed in combination with a marker coding region, one may employ the separate coding regions on either the same or different DNA segments for transformation. In the latter case, the different vectors are delivered concurrently to recipient cells to maximize cotransformation.

### II. GENETIC TRANSFORMATION

Additionally provided herein are transgenic plants transformed with the above-identified recombinant vector encoding a PHB, or a sequence modulating down-regulation thereof.

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, including alfalfa (Thomas et al., 1990), it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. Gateway™ and other recombination-based cloning technology is also available in vectors useful for plant transformation. In addition, Agrobacterium containing both armed and disarmed Ti genes can be used for the transformations. In those plant strains where Agrobacterium-mediated transformation is efficient, it is the method of choice because of the facile and defined nature of the gene transfer.

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

The transgenic plants of the present invention expressing heterologous PHB can be of any species. In some embodiments, the transgenic plant is a dicotyledonous plant, for example a plant used in biomass and forage crop production such as a poplar, a willow, a eucalyptus, a hemp, a Medicago sp., a Lotus sp., a Trifolium sp., a Melilotus sp., a Vinca sp., a Nicotiana sp., a Vitis sp., a Ricinus sp., or a Glycine sp. The plant can be an R₀ transgenic plant (i.e., a plant derived from the original transformed tissue). The plant can also be a progeny plant of any generation of an R₀ transgenic plant, wherein the transgenic plant has the nucleic acid sequence from the R₀ transgenic plant.

Seeds of the any above-described transgenic plants may also be provided, particularly where the seed comprises the nucleic acid sequence. Additionally contemplated are host cells transformed with the above-identified recombinant vector. In some embodiments, the host cell is a plant cell.

Also contemplated herein is a plant genetically engineered to increase expression of a PHB, where the PHB comprises a protein product of SEQ ID NOs:1-10, where the protein product (e.g. a polypeptide) alters plant morphology. In an embodiment, the altered plant morphology may be increased plant biomass or increased seed yield. Such plants are described in the Examples, and may be useful, e.g., as commercial plants, due to their increased plant size and seed number.

The plants of these embodiments having increased or enhanced expression of PHB may be of any species. The species may be any monocotyledonous or dicotyledonous plant, such as those described herein. One of skill in the art will recognize that the present invention may be applied to plants of other species by employing methods described herein and others known in the art.

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. A medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. The rate of cell growth also will vary among cultures initiated with the array of media that permit growth of that cell type.

Tissue that can be grown in a culture includes meristem cells, Type I, Type II, and Type III callus, immature embryos and gametic cells such as microspores, pollen, sperm, and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

### III. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce, into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad-spectrum herbicide bialaphos. Another example of a herbicide which is useful for selection of transformed cell lines in the practice of the invention is the broad-spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived therefrom. U.S. Pat. No. 4,535,060 describes the isolation of EPSPS mutations which confer glyphosate resistance on the EPSPS of Salmonella typhimurium, encoded by the gene aroA. The EPSPS gene from Zea mays was cloned and mutations similar to those found in a glyphosate resistant aroA gene were introduced in vitro. Mutant genes encoding glyphosate resistant EPSPS enzymes are described in, for example, International Patent WO 97/4103.

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 weeks, then transferred to media conducive to maturation of embryoids. Cultures are transferred every 2 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soilless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated in from about 6 weeks to 10 months after a transformant is identified, depending on the initial tissue. During regeneration, cells are grown on solid media in tissue culture vessels. Illustrative embodiments of such vessels are Petri dishes and Plant Cons. Regenerating plants can be grown at about 19 to 28° C. After the regenerating plants have reached the stage of shoot and root development, they may be transferred to a greenhouse for further growth and testing.

To confirm the presence of the exogenous DNA or “transgene(s)” in the regenerating plants, a variety of assays may be performed. Such assays include, for example, “molecular biological” assays, such as Southern and northern blotting and PCR™; “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot northern hybridizations. These techniques are modifications of northern blotting and will only demonstrate the presence or absence of an RNA species.

The expression of a gene product is often determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes that change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### IV. EVALUATION OF BIOMASS PRODUCTION

A plant useful for the present invention may be an R₀ transgenic plant. Alternatively, the plant may be a progeny plant of any generation of an R₀ transgenic plant, where the transgenic plant has the nucleic acid sequence from the R₀ transgenic plant.

The plants overexpressing PHB may also be used to produce increased plant biomass, for example by obtaining the above-identified plant overexpressing PHB, growing said plant under plant growth conditions to produce plant tissue from the plant; and preparing biomass from said plant tissue. The biomass can be subsequently used for any purpose, for example to produce biofuel.

### V. BREEDING PLANTS OF THE INVENTION

In addition to direct transformation of a particular plant genotype with a construct prepared according to the current invention, transgenic plants may be made by crossing a plant having a selected DNA of the invention to a second plant lacking the construct. For example, a selected PHB coding sequence can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein, the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a plant of a starting line with a plant of a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. DEFINITIONS

Expression: The combination of intracellular processes, including transcription and translation, undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found. In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell in which the DNA complement has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Plant Materials and Growth Conditions

All Arabidopsis plants were in the ecotype Col-0 background. Seeds were sown on soil and stratified at 4° C. for 2 days. Plants were grown at 23° C. under a medium-day photoperiod (12 h of light, 12 h of dark) in a growth chamber (light intensity ˜150 μE/s·m).

### Example 2

### Constructs and Stable Transformation

The full-length coding sequence of the Arabidopsis thaliana PHB8 gene was cloned into vector pEARLYGATE100 using the Gateway® recombination cloning system (Invitrogen). Agrobacterium strain GV3101 containing gentamycin and rifampicin resistant genes was used for stable transformation. The standard floral dipping method was performed (Zhang et al., Nat Protocol, 1(2):641-646, 2006). Briefly, approximately 20-30 healthy plants with inflorescences and some mature siliques were selected. After clipping off the siliques, the aerial parts of the plants were dipped in Agrobacterium suspension with 5% sucrose and 0.02% Silwet L-77 (OD=0.8) for approximately 20 seconds. Dipped plants were then placed under a cover to maintain high humidity for 16 to 24 hours. Seeds were collected after one month of normal growing conditions and dried for one week. Transformed seeds were selected on MS medium plates with 10 mg/L Basta. Seedlings were transplanted into soil and allowed to grow. PCR and RT-PCR were used to confirm the over-expression transgenic lines.

### Example 3

### Protoplast Transient Expression Assay

The full-length PHB8 gene and mitochondrial marker gene coding sequences (Nelson et al., Plant J, 51:1126-1136, 2007) were cloned into rebuilt vectors pX-DG and pX-DR (Chen et al., Plant Physiol, 150:1111-1121, 2009) driven by the CaMV 35S promoter. The GFP and RFP fusion constructs were transiently transformed into Arabidopsis protoplasts using the protocol described previously (Yoo et al., Nat Protocol, 2:1565-1572, 2007). Arabidopsis leaves were digested in cellulose/macroenzyme solution for more than 3 hours in the dark. The protoplasts released were transformed with PEG/Ca at room temperature for 30 seconds. Localization of fusion proteins were observed with a confocal microscope.

### Example 4

### Measurement of ATP

Sterilized seeds were plated on MS medium and stratified for 2 days at 4° C. After growing in dark at 23° C. for 2 weeks, albino seedling leaves were collected. For ATP extraction, the leaves were ground in liquid nitrogen and resuspended in TCA solution. ATP was measured by the ATP ENLITEN® ATP Assay System Bioluminescence Detection Kit (Promega) according to the manufacturer's instructions.

### Example 5

### Organelle Enrichment and Protein Isolation

For proteomic analysis of a PHB8 over-expression line, the organelle enrichment procedure was developed based on previous methods (Santoni, Meth Molec Biol, 355:93-109, 2007) with some modification. Five grams of fresh aerial tissue of Arabidopsis PHB8 over-expression plants were collected and washed in ice-cold water to remove the soil. A blender was used to disrupt the tissue after adding a 2:1 (mL medium/g fresh weight) homogenization buffer (50 mM TRIZMA base, 500 mM Sucrose, 10% Glycerol, 20 mM EDTA-Na2, 20 mM EGTA, 50 mM NaF, 5 mM beta-glycerophosphate, 1 mM phenantroline, 0.6% PVP40, 10 mM ascorbic acid, 1 mM leupeptin, 5 mM DTT, 1 mM Na-orthovanadate, pH 8.0, adjusted with MES). The homogenate was then filtered through Miracloth to remove plant debris. Centrifugation of the filtered homogenate was conducted at 1000×g for 5 minutes to remove the nuclei. The supernatant was then centrifuged at 26000×g for 25 minutes to pellet organelles.

For protein isolation of enriched organelles, pre-cooled methanol with protease inhibitor was added to the organelle-enriched pellet collected after the centrifugation. The sample was then vortexed for 30 seconds. The mixture was incubated at −20° C. then centrifuged at 16000×g for 5 minutes at 4° C. The supernatant was removed and the pellet was washed twice in methanol solution. The resulting pellet was again washed in pre-cooled acetone and centrifuged at 16000×g for 5 minutes at 4° C. Residual acetone was removed by SpeedVac, and Reagent Type 4 Working Solution was used to incubate the pellet for 15 minutes at room temperature. The pellet was then centrifuged at 16,000×g for 30 minutes, and the supernatant was collected and stored at −80° C. for future proteomics use.

### Example 6

### MudPIT Proteomic Analysis

MudPIT-based shot-gun proteomics was carried out to analyze each sample. Approximately 100 μg of protein was digested with Trypsin Gold, Mass Spectrometry Grade (Promega, Wis., USA) with 1:40 w/w at 37° C. for 24 h. The digested peptides were desalted using a Sep-Pak plus C18 column (Waters Limited, ON, Canada) and then loaded onto a biphasic (strong cation exchange/reversed phase) capillary column using a pressure tank. The 2D back column was composed of 5 cm of C18 reverse-phase (RP) resin and 3 cm of strong cation exchange (SCX) resin. The back column was then connected to a 15-cm-long 100 μm-ID C18 column (packed in-house with the same C18 reverse phase in the back column) and sprayed through a SilicaTip (New objective, Inc. Woburn, Mass.). The two-dimensional liquid chromatography (LC) separation and tandem mass spectrometry conditions followed the protocols previously described (Washburn et al., Nat Biotechnol, 19(3):242-247, 2001). Before SCX separation, a 1-h RP gradient from 100% Solvent A (95% H2O, 5% ACN, and 0.1% formic acid) to 100% Solvent B (30% H₂O, 70% ACN, and 0.1% formic acid) was configured to move peptides from C18 resin to SCX resin in the back column. The SCX LC separation was performed with 11 salt pulses containing increasing concentrations of ammonium acetate. Each salt pulse was followed by a 2-h reverse-phase gradient from 100% Solvent A to 60% Solvent B. The LC eluent was directly nanosprayed into a linear ion trap mass spectrometer, Finnigan LTQ (Thermo Fisher Scientific, San Jose, Calif.). The mass spectrometer was set to the data-dependent data acquisition mode, and full mass spectra were recorded on the peptides over a 300-1700 m/z range, followed by five tandem mass (MS/MS) events for the most abundant ions from the first MS analysis. The Xcalibur data system (Thermo Fisher Scientific, San Jose, Calif.) was used to control the LC-LTQ system and collect the data.

### Example 7

### Statistical Data Analysis of Mass Spectra

Tandem mass spectra were extracted from the raw files and converted into an MS2 file. The MS2 file was searched against the Arabidopsis protein database downloaded from The Arabidopsis Information Resource (TAIR), which contains reverse sequence and common contaminant proteins. A DBDigger algorithm was used for searching. The validity of peptide/spectrum matches was assessed in DTASelect2.0 using a 0.05 false discovery cutoff, with a cross-correlation score (XCorr) larger than 1, and a normalized difference in cross-correlation scores (DeltaCN) larger than 0.08. Proteins with more than two peptides were identified as detected and were recorded.

### Example 8

### PHB8 Over-Expression Lines Show an Increased in Biomass

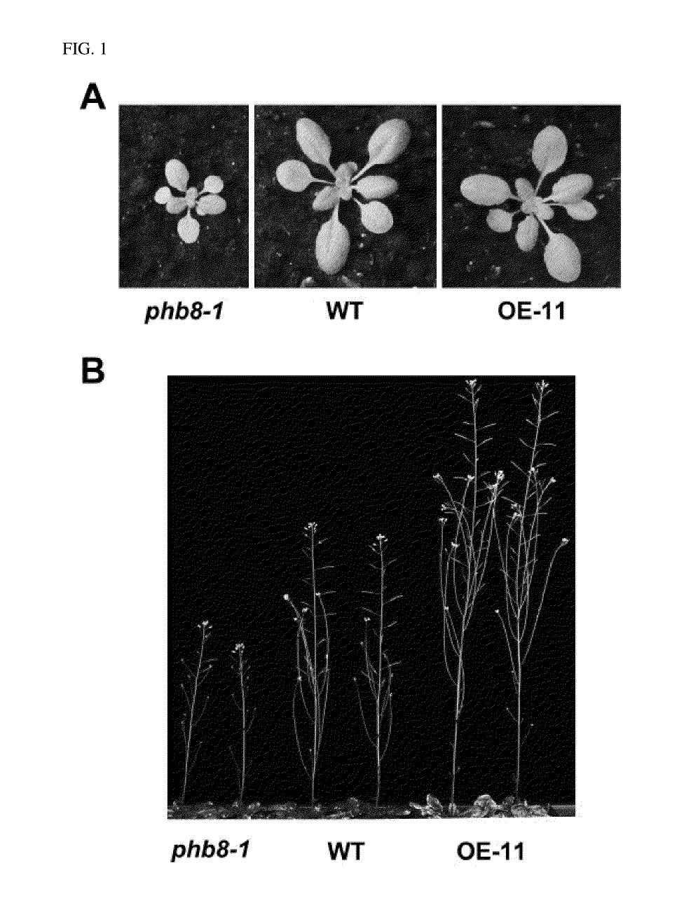

An Arabidopsis T-DNA mutant line that is smaller and exhibits delayed flowering than wild-type (WT) was identified (FIG. 1). Southern blot analysis revealed that this line is the result of a single-copy T-DNA insertion at the third exon of PHB8, a member of the prohibitin gene family (Di et al. 2010). Thus, the mutant was designated phb8-1. RT-PCR confirmed phb8-1 to be a knock-out mutant.

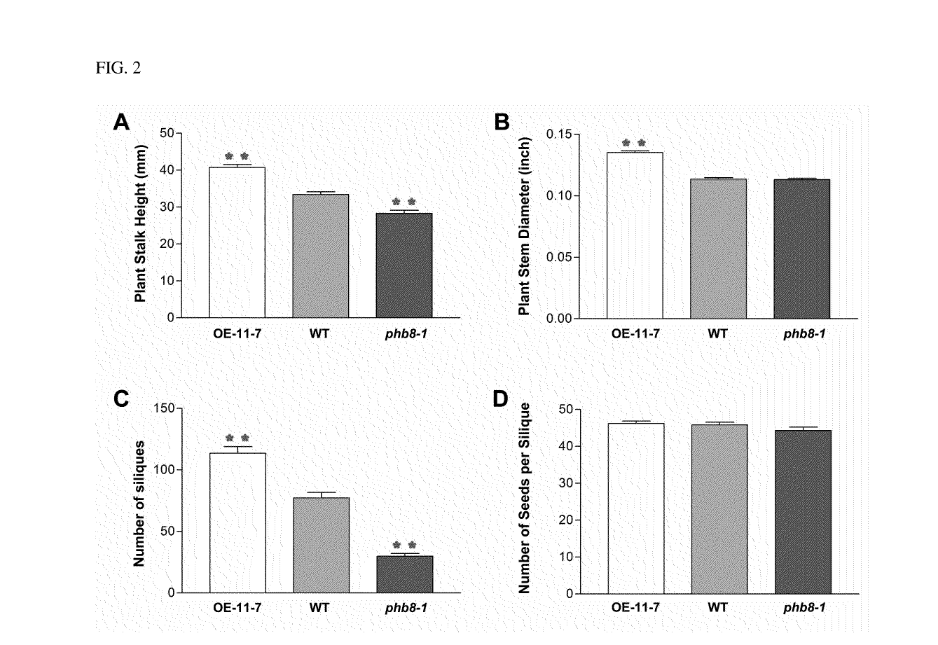

To determine the function of PHB8, the full-length cDNA controlled by the 35S promoter was introduced into WT plants. Two independent over-expression lines, OE-4 and OE-11, were identified by qRT-PCR. The bolting time of the two over-expression lines appeared earlier than WT (FIG. 1B), and the over-expression lines exhibited a significant increase in biomass yield. The stem height and diameter of 60-day-old OE-11 plants was measured and determined to be increased by approximately 20% and 19%, respectively, over WT plants. Conversely, stem height in phb8-1 plants was decreased by approximately 15%, although the stem diameter was similar to WT (FIG. 2A-B). To investigate the seed yield, the total number of siliques per plant were counted. OE-11 plants produced approximately 46% more siliques, while phb8-1 plants produced approximately 60% fewer siliques, when compared to WT plants (FIG. 2C). Further, the number of seeds per silique showed no obvious differences between WT, phb8-1, and OE-11 plants (FIG. 2D), indicating that the PHB8 over-expression line produced more seeds than WT.

### Example 9

### PHB8 Localizes to Mitochondria

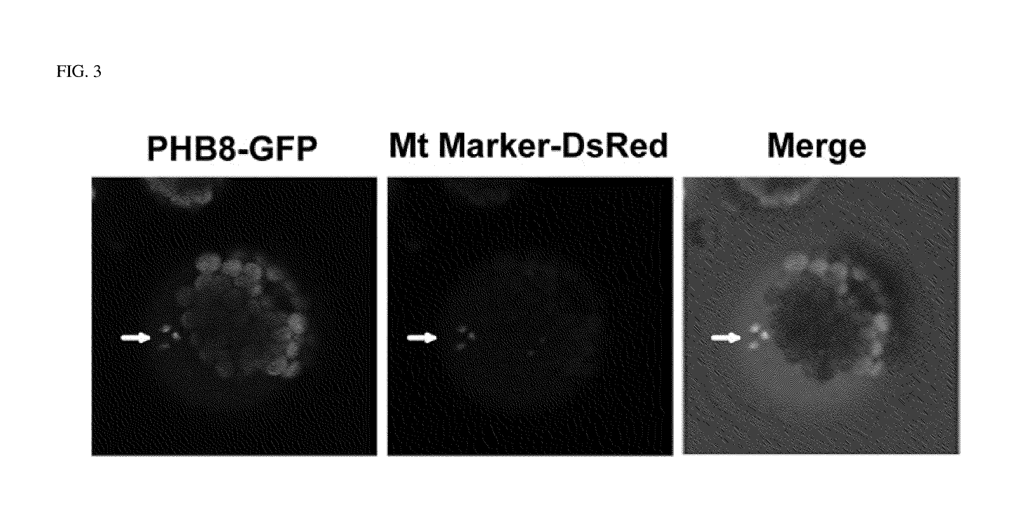

To determine the subcellular localization of PHB8, PHB8-GFP (green fluorescent protein) fusion protein was generated and transiently transformed into Arabidopsis protoplasts. A fluorescent signal was detected at the mitochondria under normal conditions. The results were supported by co-localization of PHB8-GFP and mitochondrial marker fusion RFP (FIG. 3).

### Example 10

### Proteomic Analysis of PHB8 Over-Expression Line and Wild Type

Based on the mitochondrial localization of PHB8 and the increasing biomass phenotype of PHB8 over-expression lines, a mitochondrial-enriched proteomic study was conducted to investigate proteome change between the PHB8 over-expression line and WT. Statistical analysis indicated significant expression change for many proteins (Table 1). The ATP synthase beta chain protein (AT5G08680, SEQ ID NO:12) showed a 275-fold up-regulation in the over-expression line. AT5G08680 is an F1/F0 type ATPase, which produces ATP through a proton gradient. Another up-regulated protein in mitochondria was an NAD(P)H dehydrogenase (AT1G07180). This result indicated that the PHB8 over-expression line may function in stabilizing proteins in the mitochondrial electron transportation chain to produce more ATP.

TABLE 1
Top 10 up-regulated and down-regulated proteins
for PHB8 over-expression lines

| | Fold | | |
|---|---|---|---|
| Locus | Change | pValue | Description |
| AT5G08680.1 | 274.8 | 0.000772 | ATP synthase beta chain, |
| | | | mitochondrial, putative |
| | | | (SEQ ID NO: 12) |
| AT4G20890.1 | 77.9 | 4.76E−05 | tubulin beta-9 chain |
| AT3G11250.1 | 10.09 | 0.00934 | 60S acidic ribosomal protein P0 |
| AT1G07180.1 | 7.49 | 0.011903 | Internal NAD(P)H dehydrogenase |
| | | | in mitochondria |
| AT3G01290.1 | 5.2 | 0.000117 | band 7 family protein |
| AT2G40290.1 | 5.1 | 0.022288 | eukaryotic translation initiation |
| | | | factor 2 subunit 1 |
| AT4G19120.1 | 4.9 | 0.018542 | early-responsive to dehydration |
| | | | stress protein |
| AT3G09740.1 | 4.7 | 0.026761 | syntaxin 71 |
| AT5G61970.1 | 4.4 | 0.004326 | signal recognition particle-related/ |
| | | | SRP-related |
| AT2G30620.1 | 4.2 | 0.015241 | histone H1.2 |
| AT1G29965.1 | −4.0 | 0.006277 | 60S ribosomal protein L18A |
| AT4G16150.1 | −4.1 | 0.008749 | calmodulin-binding protein, |
| AT3G16530.1 | −4.3 | 0.0305 | legume lectin family protein |
| AT5G42220.1 | −4.4 | 0.002328 | ubiquitin family protein |
| AT4G30490.1 | −4.5 | 0.005992 | AFG1-like ATPase family protein, |
| AT1G56190.1 | −4.7 | 0.01118 | phosphoglycerate kinase, putative |
| AT5G39830.1 | −4.7 | 0.001504 | DegP protease, putative |
| AT3G17170.1 | −5.0 | 0.014827 | ribosomal protein S6 family |
| AT5G47930.1 | −5.5 | 0.000126 | 40S ribosomal protein S27 |
| AT1G02930.1 | −6.3 | 0.001052 | glutathione S-transferase, putative |
| AT5G26860.1 | −6.4 | 0.010671 | similar to Lon protease, putative |

### Example 11

### PHB8 Over-Expression Lines Exhibit a Higher ATP Level

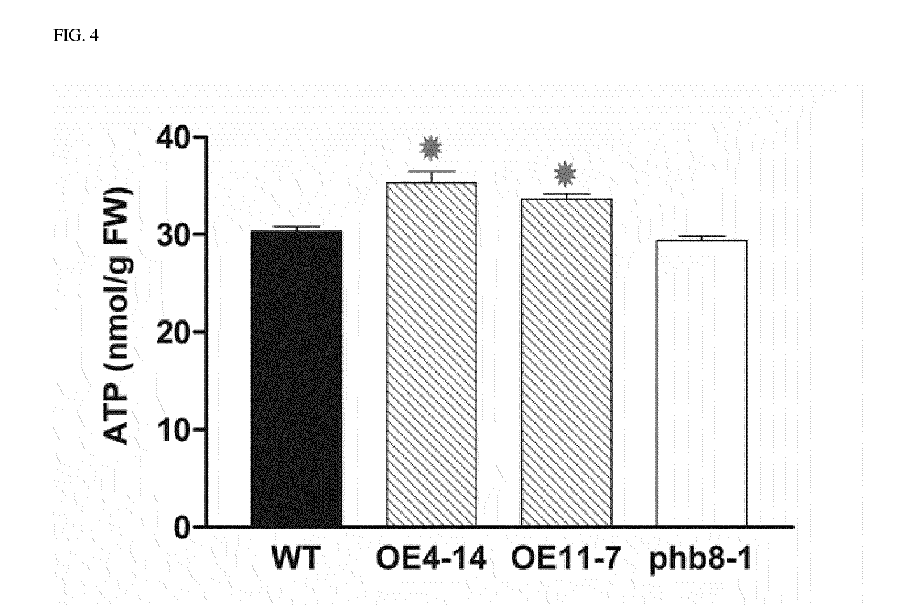

Proteomic data analysis revealed that ATPase was enriched in PHB8 over-expression lines. Results indicated that the ATP level may have some difference between over-expression lines and WT. Thus, the ATP levels in WT, phb8-1, and two over-expression lines were measured. ATP levels in OE-4 and OE-11 lines were found to be approximately 16% and 11% higher than WT, respectively, while phb8-1 plants exhibited a decrease in ATP levels of approximately 2.9% compared to WT (FIG. 4).

### Example 12

### ATPase Over-Expression in Arabidopsis

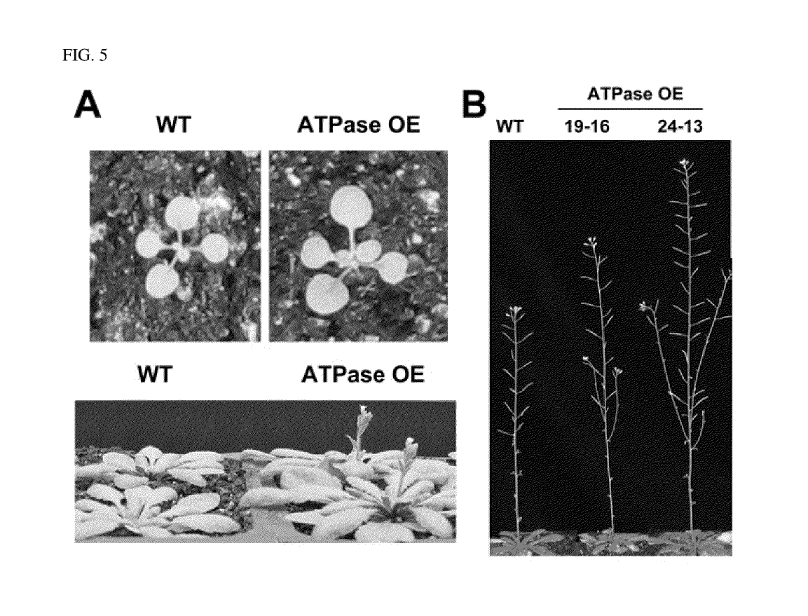

As described above, ATPase was found to be highly up-regulated in a proteomics study. An ATPase over-expression construct was transformed into Arabidopsis and the phenotype was observed. As shown in FIG. 5, the Arabidopsis line over-expressing F0/F1-type ATPase showed rapid growth (FIG. 5A, upper), early flowering (FIG. 5A, lower), and increased stem height (FIG. 5B). The similar phenotypes observed for ATPase over-expressing lines and PHB8 over-expressing lines corroborates the regulation of F0/F1-type ATPase by PHB8 in Arabidopsis.

### Example 13

### Insecticidal Properties of PHB Over-Expression in Rice

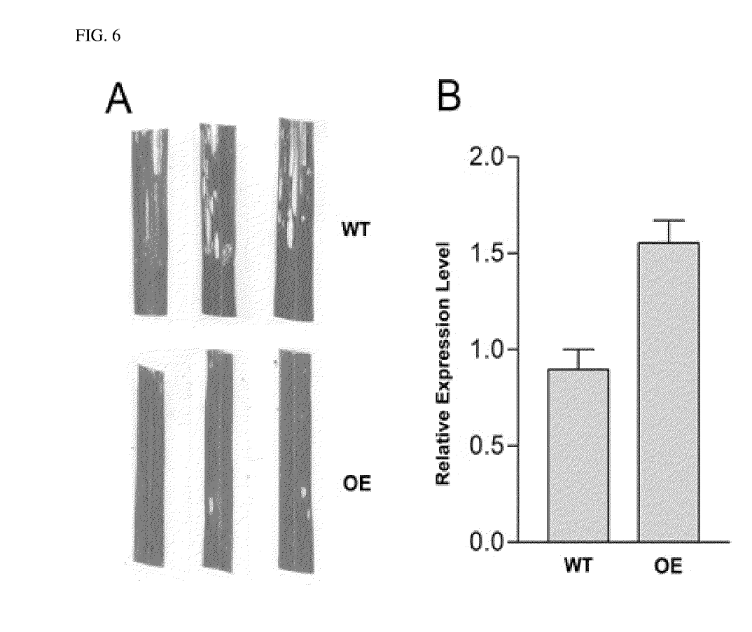

Over-expression analyses for PHB8 rice homologs in Nipponbare rice (Oryza sativa japonica) were conducted. Insect treatments were carried out by feeding WT and PHB8-over-expressing rice plants to fall armyworm larvae. Middle fragments of the third leaf were cut and inserted into 0.7% agarose to maintain moisture. One 3rd instar fall armyworm larva was put on the leaf and kept in a 28° C. growth chamber with a 14/10-h light cycle. Larvae were removed from the leaves 48 hours after inoculation. The death rate of larvae fed on leaves of WT rice was approximately 20%, compared to approximately 90% for larvae fed on leaves of PHB8 over-expression lines during the 48 hour incubation. As shown in FIG. 6, leaf area damage was greater for WT plants (15%) compared to PHB8 over-expression transgenic rice (2%), indicating that PHB8 homology in rice is related to insect defense.

What is claimed is:

1. A method of altering plant biomass comprising increasing expression of a PHB8 gene in a plant, wherein the biomass of the plant is increased when compared to a plant that lacks the increased expression.

2. The method of claim 1, wherein the plant is a dicotyledonous plant.

3. The method of claim 2, wherein the dicotyledonous plant is selected from the group consisting of Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp.

4. The method of claim 1, wherein the plant is a monocotyledonous plant.

5. The method of claim 4, wherein the monocotyledonous plant is selected from the group consisting of maize, wheat, rice, sorghum (Sorghum bicolor), oats, barley, sugar cane, African oil palm (Elaeis guineensis), or switchgrass.

6. The method of claim 1, wherein the plant has altered morphology when compared to a plant that lacks the increased expression.

7. The method of claim 6, wherein the altered morphology is increased plant biomass.

8. The method of claim 6, wherein the altered morphology is increased seed yield.

9. A plant comprising increased expression of a PHB8 gene, wherein the biomass of the plant is increased when compared to a plant that lacks the increased expression.

10. A seed that produces the plant of claim 9.

11. A seed produced by the plant of claim 9.

12. A DNA-containing plant part of the plant of claim 9.

13. The plant part of claim 12, further defined a protoplast, cell, meristem, root, leaf, node, pistil, anther, flower, seed, embryo, stalk or petiole.

14. A method of altering seed yield in a plant, the method comprising increasing expression of a PHB8 gene in the plant, wherein the seed yield of the plant is increased when compared to a plant that lacks the increased expression.

15. The method of claim 14, wherein the plant is a dicotyledonous plant.

16. The method of claim 15, wherein the dicotyledonous plant is selected from the group consisting of Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp.

17. The method of claim 14, wherein the plant is a monocotyledonous plant.

18. The method of claim 17, wherein the monocotyledonous plant is selected from the group consisting of maize, wheat, rice, sorghum (Sorghum bicolor), oats, barley, sugar cane, African oil palm (Elaeis guineensis), or switchgrass.

19. The method of claim 14, wherein the plant has altered morphology when compared to a plant that lacks the increased expression.

20. The method of claim 19, wherein the altered morphology is increased plant biomass.

21. The method of claim 19, wherein the altered morphology is increased seed yield.

22. A plant comprising increased expression of a PHB8 gene, wherein the seed yield of the plant is increased when compared to a plant that lacks the increased expression.

23. A seed that produces the plant of claim 22.

24. A seed produced by the plant of claim 22.

25. A DNA-containing plant part of the plant of claim 22.

26. The plant part of claim 25, further defined a protoplast, cell, meristem, root, leaf, node, pistil, anther, flower, seed, embryo, stalk or petiole.

27. A method of producing increased plant biomass, the method comprising:
(a) obtaining a plant comprising increasing expression of a PHB8 gene, wherein the biomass of the plant is increased when compared to a plant that lacks the increased expression;
(b) growing said plant under plant growth conditions to produce plant tissue from the plant; and
(c) preparing biomass from said plant tissue.

28. The method of claim 27, wherein preparing biomass comprises harvesting said plant tissue.

29. The method of claim 27, further comprising using the biomass for biofuel.

30. A method for protecting a plant from pest infestation comprising:
(i) increasing expression of a PHB8 gene in a plant; and
(ii) providing in the diet of a plant pest an insecticidally effective amount of cells or tissue of the plant or part thereof;
wherein the insecticidal properties of the plant are increased when compared to a plant that lacks the increased expression.

31. The method of claim 30, wherein the plant is a dicotyledonous plant.

32. The method of claim 31, wherein the dicotyledonous plant is selected from the group consisting of Arabidopsis, peanut (Arachis hypogaea), barrel medic (Medicago trunculata), carrot, soybean (Glycine max), cotton, Brassica, canola, tomato, potato, alfalfa, grape, clover, poplar, willow, eucalyptus, hemp, a Lotus sp., a Vinca sp., a Nicotiana sp., a Vitis sp., or a Ricinus sp.

33. The method of claim 30, wherein the plant is a monocotyledonous plant.

34. The method of claim 33, wherein the monocotyledonous plant is selected from the group consisting of maize, wheat, rice, sorghum (Sorghum bicolor), oats, barley, sugar cane, African oil palm (Elaeis guineensis), or switchgrass.


SEQUENCE LISTING (195 sequences)

SEQ ID NO: 1 - DNA, 858 bp, Arabidopsis thaliana
atggggaatcttttctgttgcgtgcttgtgaagcaatcagatgttgcggtcaaggagagatttggcaaattccaaaaagtacttaatccaggtctccaatttgttccatgggtcatcggtgattacgtcgccggtacactgacccttcgtcttcagcaactcgatgttcagtgtgaaaccaaaacaaaggacaatgtgtttgtgacagtggttgcatccatacaatacagagtcttagctgacaaggcaagtgatgctttttacagactcagcaatccaaccacccaaatcaaagcctacgtctttgatgtgatcagagcatgtgttccaaagctgaacttggacgatgtgttcgagcagaagaatgaaattgccaaatctgtggaagaagagctagacaaagccatgactgcttatggttacgaaatccttcaaaccctaattatcgacattgagcctgatcaacaggttaaacgtgccatgaacgaaatcaacgccgcggcgaggatgagagtggcagcgagcgaaaaagcagaggctgagaaaatcattcagatcaaaagagcagagggtgaagcagagtcaaagtacctgtcgggactcggaatcgctcggcagagacaagcgatcgtggacggtctgagagacagtgttctagggttcgcaggaaacgtgccagggacgtcagcgaaggatgtgttggacatggtgatgatgactcagtactttgacacaatgagagatatcggagcaacttctaaatcctctgcggtgtttatccctcacggtccaggcgccgtctctgacgtggcagcgcagattcgaaatggattattacaggccaacaatgcctcctaa

SEQ ID NO: 2 - DNA, 879 bp, Oryza sativa
atgggaaaattggtggcagcaataggtaaattgctctgctgtgtacaagttgaccagtcaacagtaggcatcaaggaaagatttgggaaatatgaggaggtgcttgatcctggatgtcactgcgtgccatggattattggaagccgtgttgctggtgagcttacactaaggcttaggcagctagatgtacgttgtgagacaaagacaaaggacaatgtttttgttacggttgttgcatccattcaatatcgagcaatggaggacaaagcaagcgatgcatactacaagctgagcaacccaaaatcccaaatccaatcttatgtctttgatgttatcagggcaagtattcccaaactagaattggacgatgcttttctgcaaaagaatgaaatagcaagagctgtggaggaggagcttgagaaggcaatgctagcttatggttatgagattgtgcaaacactgattgttgacatagaaccagatgagaaagttaagagggcgatgaacgaaattaatgctgctgcaaggctacgtgttgcagcaaatgagaaggcagaggcagagaagatcattcagatcaagagggccgagggagaggctgaagccaagtatctgtctgggcttggtattgcaaggcagcggcaggctatagttgatgggctaagagacagtgtacttggcttctctggcaatgttcctgggacttcagctaaggatgtgatggatttagtcctgctgacgcagtactttgacactatgaaagagatcggttcaacatccaaatcttcagcaattttcctccctcatggccctggtgctgttgcagatattgccagtcagatccgtgatggatgtcttcaggctcatcaaacaaagtga

SEQ ID NO: 3 - DNA, 855 bp, Zea mays
atgggtcagattttgggtttagttcaggttgatcagtcaactgtagccatcaaagaaaattttggcaagttcagcgaggtcctggagcctggttgccacttcctgccctggtgcatagggcagcagattgctggttacctctccttgcgtgtgcgccagctggatgtccgctgcgaaacaaagacaaaggacaatgtctttgtcactgttgttgcatctgttcaatatcgcgctcttgctgataaggcatctgacgccttctacaagctgagcaacaccagggaacaaatccagtcatacgtatttgatgtcatcagagctactgttccgaagctgggcttggacgatgcatttgagcagaagaatgagatcgcaaaagcagtggaagaggagcttgaaaaggcaatgtctacttatggctatcagatcgtgcaaacgctaattgttgatattgagcctgacgaccgcgtcaagagagcaatgaacgagatcaatgcagctgctaggatgagggtggcagccagtgagaaagctgaggctgagaagatactccagatcaagaaagccgagggagaggcggaatccaagtacctggctggcgtgggtattgcaaggcagcgccaggccattgtggatgggctgagggacagtgtgctcgccttctcagagaacgttccaggcaccactgccaaggacatcatggacatggtcctggtcacccagtactttgacaccatgagggagattggggcctcctccaagtcctcttcggtgttcatcccccacggtcctggagctgtcaaggatgtctcggcgcagataagagatggcctcctgcaggctaatctgcactga

SEQ ID NO: 4 - DNA, 855 bp, Sorghum bicolor
atgggtcaggctttgggtttagtgcaggttgatcagtcaactgtagccatcaaagaaaacttcggcaagttcagtgaggtcctggagcctggttgccacttcctgccctggtgcatcgggcagcagattgctggttacctctccttgcgtgtgcgccagctggatgtccgctgtgaaacaaagacaaaggacaatgtctttgtcactgttgttgcatctgtccaatatcgtgctctcgctgaaaaggcatctgacgccttctacaagctgagcaacactagggaacaaattcagtcgtatgtatttgatgtcatcagagctactgttccaaagctggacttggacgatgcatttgagcagaagaatgacatcgcgaaagcagtcgaagaggagcttgaaaaggcaatgtctatgtacggctatgagatagtgcaaacgctaattgttgatattgagcctgatgaccgtgtcaagagagcaatgaacgaaatcaatgcagctgctagaatgagggtggcagccagtgagaaagctgaggctgagaagatactccagatcaagaaagccgaaggagaggcggaatccaagtacctggctggtgttggtattgcaaggcagcgccaggccattgtggacgggctgagggacagtgtgctcgccttctcagagaacgttccaggcaccactgccaaggacatcatggacatggtcctggtcacccagtactttgacaccatgaaggagattggggcctcctccaagtcctcttcagtgttcatcccccatggccctggagctgtcaaggatgtcgcggcgcagataagggatggcctcctgcaggctaaactgcagtga

SEQ ID NO: 5 - DNA, 855 bp, Glycine max
atggggaatcttttttgttgtgtgcaagttgatcaatcaacagtggctataagagaaggatttggacgatttgagaaggtacttcagccaggatgccattgcatgccatggttccttggaaaacaacttgctggtcatctctctcttcggctacagcaactggatcttcgatgtgagaccaaaacaaaggataatgtctttgtcaacgttgttgcttctattcaatatcgtgccctggcagagaaggccaatgatgctttttacaaattgagcaatacaaagacccaaattcaagcctatgtttttgatgtaattagggcaagtgttccaaaactaaacttagatgatgcttttgagcagaaaaatgaaattgccaaagctgtggaagaagaacttgagaaggctatgtcagcttatgggtatgaaattgttcaaacactgattgttgatatagatccagatgagcatgtgaagcgggccatgaatgaaatcaatgctgctgcaagattgaggatggcagctaatgagaaggcagaggcagaaaagatcttgctaattaaacgagccgagggtgaggctgagtctaagtatctctctggactgggtattgctcgccaacgccaagcaattgtggatggcttgagagatagtgtgcttggattctcagttaatgtacctgggacaactgcaaaagatgtcatggacatggtccttgtcactcagtattttgacactatgaaagaaattggtgctgcctctaagtcttctgctgtgttcattccacatggacctggtgctgttcgtgatgtagctggtcaaattcgggagggacttcttcaggcttctcatcagtag

SEQ ID NO: 6 - DNA, 849 bp, Elaeis guineensis
tcattgcgtgtgcagcaacttgatgttcgttgtgaaacaaagacaaaggataatgtgtttgttactgttgttgcatctatacaataccgtgctcttgctgataaggcatctgatgctttctatagacttagcaacacaagggaacagattcaatcctatgtttttgatgtcatcagggcgagtgttccaaagctgaacttagatgatgtattcgaacagaagaatgatattgcgaaagctgtggaagatgaacttgaaaaggcaatgtcaatgtatggatatgagatagttcaaacactcattgtggatattgagcctgacgagcatgttaagagggcaatgaacgagatcaatgcagctgccaggctgagggtggcagcaaatgaaaaggctgaagctgagaaaatactgcagatcaagcgtgctgaaggagatgcagaatccaagtacttagcggggcttggtatagcacgccaacgccaggccattgtggatgggctgagggacagcgtgcttgccttctctgtaaatgtacctgggaccacagccaaggatgtcatggatatggtgctggtgacacagtactttgacaccatgaaagagattggagcatcctcaaaggcctcttctgtgttcatccctcatgggcctggggctgtgagggacattgctgcacaaataagggatggcctcctccaagctagcaccactcagcagtaggaccaaatggaggctagtttcgtggggatggattagcagtttaaaacggttgattgtttcttttcttctaaagtgctttaatttggtgccggtatttggaagatgaagcagaaaacaaaacataaggtg

SEQ ID NO: 7 - DNA, 333 bp, Arachis hypogaea
acccaaattcaagcctatgtttttgatgttattcgcgctagtgtgccaaaattagagttggatgcggtgtttgaacaaaagaatgatatagcaaagtctgttgaggaggagcttgagaaggccatgtctacctacggatatcagatagtccagaccctgattgtggatatcgaacctgacactaatgttaagagagccatgaatgagatcaatgcagctgctagactgagggtggctgcaaatgagaaagctgaagcagaaaagattctgcaaatcaagaaagctgagggagaggcagagtctaagtatctttctggtctgggtattgctcgc

SEQ ID NO: 8 - DNA, 1171 bp, Vitis vinifera
gtaatcttgtaatcgaagagcgcagaaaacatgggtaatctgcttggttgcatacaagtcgatcaatccacagtagctatcaaggagagatttggcaagtttgaggaagtgcttgagccaggatgccattgcttgccttggtgctttgggagtcagcttgctggccatctttcacttaggttgcaacagttggatgtgcgttgtgagaccaagacaaaggacaatgtgtttgtcaatgttgttgcttctatacaatatcgtgccctggcagataaggcaaatgatgctttttacaaactcagcaatacaaggtcccagatccaggcttatgtttttgatgttattagagcaagtgttccaaagctcaatctggacgatgcatttgagcaaaaaaatgaaattgctaaatctgtggaagatgaacttgaaaaggctatgtctgcctatgggtatgagattgtacaaacactcattgttgatatagaaccagatgagcatgtgaagcgagcaatgaatgaaatcaatgctgctgcaagaatgagggtggcagccaatgagaaggcggaggctgagaaaattttgcaaatcaagcgtgcagagggtgaggctgagtccaagtatctatcagggcttggtattgctcgtcagcgtcaagcaattgttgatggcttgagagacagtgtgctaggcttctcagtcaatgttccaggcaccactgcgaaggatgtgatggacatggtcctcgtcacacagtacttcgacaccatgaaggaaattggtgctgcttctaaatcctcagctgtgttcatcccccatggacccggagctgttcgtgatgtggctactcagattcgtgatggacttcttcagagtaccctctcacatcagtaattgtcttatgtttacaaggttttttctatttttaggcttgtcactgaaacaactttgatgtgaagtttatgttcatgttgatctctcagggtgtttagtgtggtttggttttatttcatggtcagtgcaatctgtccattgctattattgtacatatgaactcaaagatttgtcacatgagccgtatacttgaatttgattttaaactttgagagatttcttaatgctaatgctagtttctcctgcaagaatatattggatattggtttctgtttcacct

SEQ ID NO: 9 - DNA, 1068 bp, Ricinus communis
gcacggctttgttaattccatctgaaacggaaaacgtatttctcgctcgtcaccttttcctagaaattacaattccttgcaatcgagttttctcctcctcctctccactatatgtatatatatacatgtgtatagaattctccctcactttcgtcttcttatctccgtgtttgcgttttagtgctttttcgcttttaatctaaacagaaaatgggacaagccctaggatgtgttcaagtggaccagtcaacagttgctatcaaggaaacctttgggaaatttgatgatgtgcttgaacctggttgtcattgtttgccttggtgtttgggaagccagttagctggtcacctctctttacgtgtgcagcagcttgatgttcgatgtgaaaccaaaaccaaggacaatgtgttcgttactgtggttgcttctattcaataccgagcactggcagaaaaggctgcagatgcattttataagctcagtaataccagagcacagatccaagcctatgtatttgatgttattcgggcaagtgttccaaagttggacttggattcagcctttgaacaaaagaatgatatagcaaaagctgtggaaaatgagcttgaaaaggccatgtcacattacggatttgagatagttcagactcttattgtggatattgagccagatgagcatgtgaagagggcaatgaacgagattaatgcagctgctagaatgagggtggctgccagtgaaaaagccgaagcagagaagatattgcagattaagcgagctgagggagatgcagagtcaaaatatctggcaggtcttggtatagctaggcagcgccaggccatcgtagatgggctgagagacagtgttctagctttctccgagaatgtaccagggaccagttcgaaggatgtcatggacatggttctggtgacacagtactttgataccatgaaggagattggtgcatcctcaaaatccagctctgtcttcattcctcatggacccggtgctgtgagagatattgccacacagatcagggatggtctgcttcaggccaactcgactcggtag

SEQ ID NO: 10 - DNA, 900 bp, Medicago truncatula
atgcttcttcatctttcccttgcaacaactgaaaagaaaatggggaatcttgtgtgttgtgtgcaagttgatcaatctcaagtggctatgaaagaaggttttggaaaatttgaaaaggtgcttcagccgggatgccattgcatgccatggttccttggaaaaagaattgctggtcatctctctcttcgggtacaacaattggatatcaaatgtgagaccaagacaaaggataacgtctttgtcaatgttgttgcttctattcaataccgggccttggcagacaaggcgaatgatgcattttacaaacttagcaacacaagaaaccaaattcaagcttatgtttttgatgtaattagggcaagtgttccaaaactcaacttggatgatacttttgagcagaaaaatgaaattgcaaaagctgtggaagaagaacttgagaaggctatgtcagcttatggatatgaaattgttcaaacactgattactgatatagagccagatgtccatgtgaagcgggctatgaatgaaatcaatgcagctgcaagaatgaggttggcagctaaagagaaggctgaagcagagaaaatcttgcaaattaagcgagccgagggtgaggctgagtctaaatatctctctgggttgggtattgctcgccagcgtcaagcgattgtggatggtttgagagacagtgtgattggattttcagttaatgtaccagggacgactgcaaaagatgtcatggatatggtccttgtcactcaatactttgacactatgaaagaaattggtgctgcctccaagtcttctgctgtgttcattccacatggacctggtgctgttcgtgatgtagctagccaaattcgtgatggacttctccagggttctctatctcatcagtaa

SEQ ID NO: 11 - DNA, 1671 bp, Arabidopsis thaliana
atggcgtctcggagagtcttatcatctcttctccgttcatcttccggtagatctgccgccaaattaggcaaccgtaaccccaggcttccttctccttcacccgcgcgtcccgccgctccatgtagctacctcctcggccgcgtcgccgagtacgcgacctcttcacctgcaagctcagctgcgccatcttctgctcctgctaaggatgaggggaagaagacctatgattacggtggcaaaggtgcgatcgggcgtgtttgccaggtcattggtgccattgtcgatgtgagatttgaggatcaggaaggattgccaccaatcatgacatctctcgaggtgcaggatcaccccacaaggctagtgcttgaggtgtctcatcacttgggtcagaatgtcgtcaggaccattgctatggatggtactgagggtctcgttcgtggaaggaaagtcctcaacactggcgctccaatcactgtacctgtcggaagggctactcttggacgtatcatgaatgttcttggagaacctattgacgaaagaggcgaaattaagaccgaacattacttacctattcacagagatgcaccggctttggttgatctagctactgggcaagagatcctggccactggtattaaggttgttgatcttcttgctccttaccaaagaggaggaaagattggtctctttggcggtgctggtgttgggaaaactgtgcttattatggagctgatcaacaatgttgccaaagctcatggtggtttctctgtgtttgctggtgtgggagaacgaacccgtgaaggcaatgacttgtacagagaaatgattgagagtggtgtcatcaagctaggcgaaaagcagtctgagagcaagtgtgctctagtgtacggacaaatgaatgagcccccgggtgcccgtgcccgtgttggactgactggtttgactgttgccgagtatttccgtgatgctgaaggccaagacgttttgcttttcattgacaacattttccgtttcactcaggccaactctgaagtgtctgctttactcggtcgtatcccgtctgctgtgggataccagccaactctggcttctgatcttggtgctcttcaagagcgaattactaccaccaagaaagggtctatcacctcagtccaagccatctatgtacctgctgatgatttgactgatcctgctcctgctacaacttttgctcacttggacgccacaactgtgctctcaagacagatttctgagcttggtatctaccctgctgtggatcctttggattcaacatcccgtatgctgtcacctcacattctgggagaggagcattacaacacggctcgtggcgtgcagaaagttctacagaactacaagaacttgcaagatattattgcaattttgggtatggatgagctaagtgaagatgacaagttgactgttgcccgtgcccgtaagatccagagattcttgagtcagccgttccacgttgctgagatcttcactggtgcccctggaaaatatgtcgaccttaaagaaaatatcaacagtttccagggtttgttggatggcaagtacgatgatctttccgaacaatcgttttacatggttggaggtatcgatgaggtggttgcaaaggcagagaagatcgctaaagagtcagcagcttaa

SEQ ID NO: 12 - DNA, 1680 bp, Arabidopsis thaliana
atggcgagtcggcgaatcttatcatcgcttctccgttcttcttcgagtagatctacttctaaatcctccttgatcgggagccgaaacccgaggcttttatcccccggtcccgcgcatggagccgctccatgtgggactctccttggccgagtcgccgagtattcgacttcttctccggctaattcggctgcaccatcttctgctcctgctaaagatgagggaaagaagacctatgattacggtggcaaaggcgcgatcgggcgtgtgtgtcaggttattggtgccattgttgatgtgagattcgaagatcaggagggattgcctccgatcatgacatctctcgaggtacaggatcatccaacaaggttggtccttgaggtgtctcatcacttgggtcaaaatgtcgttaggaccattgctatggatggtactgagggtctcgtccgtggaaggaaggttctcaacactggtgctccaatcactgttcctgttggaagggccacccttggacgtatcatgaacgttcttggagaacctattgacgagagaggcgaaattaagaccgagcattacttacccattcacagagacgctccagctttggttgatttggccaccggtcaagagattctcgctactggtattaaggttgttgatctccttgctccttaccaaagaggaggaaagattgggctttttggcggtgctggtgttgggaaaacagtgctcattatggaactgattaacaatgttgccaaagctcatggtggtttctccgtgtttgctggtgtgggagaacggacccgtgaaggcaatgatttgtacagagaaatgattgagagtggtgtcatcaagctaggagagaaacagtctgagagcaaatgtgccctagtgtatggacaaatgaatgagcccccgggtgctcgtgcccgtgttggactgactggtttgactgttgctgagtatttccgtgatgctgaaggccaagatgtcttgcttttcattgacaacatcttccgtttcacccaggccaactctgaagtgtctgcgttgctcggtcgtatcccgtctgctgtggggtaccagccaactctggcttccgatcttggtgctcttcaagagcgaatcacaaccaccaagaaaggttcaattacctctgtccaagccatctatgtccctgctgatgatttgacagatcctgctcctgccacaacttttgctcacttggatgccacaactgtgctttcaagacagatttctgagcttggtatctaccctgctgtggatcctttggattcgacgtcccgtatgctctcgccccacattctaggtgaggagcactacaacacagctcgtggtgtgcagaaagtgttgcagaattacaagaatttgcaagatattatcgccattttgggaatggatgagctaagtgaagatgacaagctgactgttgcccgtgcccgtaagatccagagattcttgagtcagccattccatgttgctgaaatcttcactggtgcccctggaaaatatgtcgaccttaaagaaaacatcaacagtttccagggtttgttggatggcaagtacgatgatctttccgaacaatcgttttacatggttggaggtatcgatgaggtggttgcaaaggcagagaagatcgctaaagagtcagcagcttag

SEQ ID NO: 13 - DNA, 1671 bp, Arabidopsis thaliana
atggcgtctcgaagagttttatcatctcttctccgttcatcttccggtagatctgccgccaaattagtcaaccgaaaccccaggcttccttctccttcacccgcgcgtcacgccgctccatgtagctacctcctcggccgcgtcgccgagtatgcgacctcttcaccggcaagctctgctgcgccatcctctgctcctgctaaggatgaggggaaaaagacctatgattacggtggcaaaggtgcgatcgggcgtgtttgtcaggtcatcggtgccattgtcgatgtgagattcgaggatcaggaaggattgcctccgatcatgacatctcttgaggttcaggatcatcccacaaggctggtgcttgaggtgtctcatcatttgggtcaaaatgtcgttaggaccattgctatggatggtactgagggtcttgtccgtggaaggaaggttctcaacactggtgctccaatcactgtacctgttggaagagctacccttggccgtatcatgaatgtgcttggagaacccattgacgagagaggcgaaatcaagaccgagcattacttacctattcacagagatgctccggctttggttgatctagccactgggcaagagatcctggccactggtattaaggttgttgatcttcttgctccttaccaaagaggaggaaagattggtctctttggcggtgctggtgttgggaaaactgtgcttattatggagctgatcaacaatgttgccaaagctcatggtgggttctctgtgtttgctggtgtgggggaacgaacccgtgagggcaatgacttgtacagagaaatgattgagagtggtgtcatcaagctaggcgagaagcagtctgagagcaaatgtgccctagtgtatggacaaatgaatgagcccccgggtgctcgtgcccgtgttggactgactggtttgactgtggctgagtatttccgtgatgctgaaggccaagatgtcttgcttttcattgacaacattttccgtttcactcaggccaactctgaagtgtctgctttgctcggtcgtattccgtctgctgtgggttaccagccaactctggcttctgatcttggtgctctgcaagagcgaatcacaaccaccaagaaagggtctatcacctcagtccaagccatctatgtccctgctgatgatttgacggatcctgctcctgccacaacttttgctcacttggacgccacaaccgtgctgtcaagacagatttctgagctcggtatctatcctgctgtggatcctttggattcgacatcccgtatgctctccccccacattctgggtgaggagcactacaacactgctcgtggtgtgcagaaagtgttgcagaactacaaaaatttgcaagatattattgccattttgggaatggatgagctaagtgaagatgacaagctgactgttgcccgtgcccgtaagatccagagattcttgagtcagccattccatgttgctgagatcttcacgggtgcccctggaaaatacgttgatcttaaggaaaacatcaacagtttccagggtctactcgacggtaagtacgatgatctttccgaacaatcgttttacatggttggaggtatcgatgaggtggttgcaaaggcagagaagatcgctaaagagtcagcagcttaa

SEQ ID NO: 14 - protein, 559 aa, Arabidopsis lyrata
MASRRILSSLLRSSSGRSTSKSSLIGSRNPRLSSPGPAHRAAPCGTLLGRVAEYSTSSPANSAAPSPAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTEHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLSEQSFYMVGGIDEVVAKAEKIAKESAA

SEQ ID NO: 15 - protein, 559 aa, Capsella rubella
MASRRILSSLLRSSTGRSTSKSSLIGSRNPRLSSPGPAHRAAPCATLLGRVAEYSTSSPANSAAPSSAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTDHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLPEQSFYMVGGIDEVVAKAEKISKESAA

SEQ ID NO: 16 - protein, 556 aa, Arabidopsis lyrata
MASRRVLSSLLRSSSGRSAAKLGNRNPRLPSPSPARHAAPCSYLLGRVAEYATSSPASSAAPSSAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTEHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLSEQSFYMVGGIDEVVAKAEKIAKESAA

SEQ ID NO: 17 - protein, 556 aa, Arabidopsis thaliana
MASRRVLSSLLRSSSGRSAAKLGNRNPRLPSPSPARPAAPCSYLLGRVAEYATSSPASSAAPSSAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTEHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLSEQSFYMVGGIDEVVAKAEKIAKESAA

SEQ ID NO: 18 - protein, 556 aa, Arabidopsis thaliana
MASRRVLSSLLRSSSGRSAAKLVNRNPRLPSPSPARHAAPCSYLLGRVAEYATSSPASSAAPSSAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTEHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLSEQSFYMVGGIDEVVAKAEKIAKESAA

SEQ ID NO: 19 - protein, 621 aa, Capsella rubella
MFPTFHHQNYMSSMISILNFDLPTFLFILWIPKGQGSLFLIRQSAYVVTLAFPLCLFSISLTHLLMASRRVLSSLLRSSSGRSAAKLGNRNPRLPSPSPARHAAPCSYLLGRVAEYATASPASSAAPSSAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTDHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLPEQSFYMVGGIDEVVAKAEKISKESAA

SEQ ID NO: 20 - protein, 558 aa, Capsella rubella
MASRRVLSSLLRSSSGRSAAKLGNRNPRLPSPSPARHAAPCSYLLGRVAEYATASPASSAKDAPSSAPAKDEGKKTYDYGGKGAIGRVCQVIGAIVDVRFEDQEGLPPIMTSLEVQDHPTRLVLEVSHHLGQNVVRTIAMDGTEGLVRGRKVLNTGAPITVPVGRATLGRIMNVLGEPIDERGEIKTDHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEIFTGAPGKYVDLKENINSFQGLLDGKYDDLPEQSFYMVGGIDEVVAKAEKISKESAA

SEQ ID NO: 21 - protein, 579 aa, Eutrema salsugineum
MELRFRFCCYPSISSLSHTTNPLMASRRVLSSLLRSSSGRSAAKFGSRSPRIPSPSPARCVSPFGDLLGRVAEYSTSSPANSAAPSSAPAKDEGKKTYDYGGKGAIGKVCQVIGAIVDVRFEDQEGLPPIMTSLEVQDHPTRLVLEVSHHLGQNVVRTIAMDGTEGLVRGRRVLNTGAPITVPVGRATLGRIMNVLGEPIDERGEIKTDHYLPIHRDAPALVDLATGQEILATGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEYFRDAEGQDVLLFIDNIFRFTQANSEVSALLGRIPSAVGYQPTLASDLGALQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKLTVARARKIQRFLSQPFHVAEIFTGAPGKYVDLKENINSFQGLLDGKYDDLPEQSFYMVGGIDEVVAKAEKISKESAA

SEQ ID NO: 22 - protein, 562 aa, Solanum lycopersicum
MASRRLIASLLRSSAQRGAGSGGGPISRSSVANSIARPASRASPKGFLLNRAVQYATSAAAPASKPSTPPKSSGSEVSGKITDEFTGAGAIGKVCQVIGAVVDVRFDDGLPPILTALEVLDNQIRLVLEVAQHLGENMVRTIAMDGTEGLVRGQRVLNTGSPITVPVGRSTLGRIMNVIGEAIDERGPITTDHFLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKMTVARARKIQRFLSQPFHVAEVFTGAPGKYVDLKESINSFQGVLDGKYDDLSEQSFYMVGGIDEVIAKAEKIAKESAA

SEQ ID NO: 23 - protein, 556 aa, Solanum tuberosum
MASRRLMASLLRSTAQRGGAISRSPLANSIPKTTSRASPAGFLLNRAVKYATSAAPADKSATPPKSSGNEPSGKITDEFTGAGSVGKVCQVIGAVVDVRFDEGLPPILTALEVLDNQIRLVLEVAQHLGENMVRTIAMDGTEGLVRGQRVLNTGSPITVPVGRSTLGRIMNVIGEPIDERGDITTDHFLPIHREAPAFVEQATEQEILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKMTVARARKIQRFLSQPFHVAEVFTGAPGKYVDLKESINSFQGVLDGKYDDLSEQSFYMVGGIEEVIAKAEKIAKESAA

SEQ ID NO: 24 - protein, 557 aa, Solanum lycopersicum
MASRRLMASLLRSTAQRGGAISRSPLANSIPKATSRASPAGFLLNRAVKYATSAAAPADKSATPPKSSGNEPTGKITDEFTGAGAVGKVCQVIGAVVDVRFDEGLPPILTALEVLDNQIRLVLEVAQHLGENMVRTIAMDGTEGLVRGQRVLNTGSPITVPVGRSTLGRIMNVIGEPIDERGDITTDHFLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKMTVARARKIQRFLSQPFHVAEVFTGAPGKYVDLKESINSFQGVLDGKYDDLSEQSFYMVGGIEEVIAKAEKIAKESAA

SEQ ID NO: 25 - protein, 562 aa, Solanum tuberosum
MASRRLIASLLRSSAQRGAGSGGGPISRSSIANSIARPASRASPKGFLLNRAVQYATSAAAPASKPSTPPKSSDSEASGKITDEFTGAGAIGKVCQVIGAVVDVRFDDGLPPILTALEVLDNQIRLVLEVAQHLGENMVRTIAMDGTEGLVRGQPVLNTGSPITVPVGRSTLGRIMNVIGEAIDERGPITTDHFLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKMTVARARKIQRFLSQPFHVAEVFTGAPGKYVDLKESINSFQGVLDGKYDDLSEQSFYMVGGIDEVIAKAEKIAKESAA

SEQ ID NO: 26 - protein, 558 aa, Fragaria vesca
MASRRLLSTLARSSVRRSPSKSPISNSHPRLTSPSPPPHRASPYGYLLSRVAEYATAAAAETKSVSPAGPKDGAKGKITDEFTGKGAIGQVCQVIGAVVDVRFEEGLPPILTALEVLDNSIRLVLEVAQHLGESMVRTIAMDGTEGLVRGQRVLNTGSPITVPVGRSTLGRIMNVIGEPIDHRGDITTEHYLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQADSKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEVFTGAPGKYVELKESIQSFQGVLDGKFDDLSEQSFYMVGGIEEVIAKAEKIAKENA

SEQ ID NO: 27 - protein, 559 aa, Glycine max
MASRRFVSSLIRSSLRRSQSKPSISASASRLTSSNRASPHGYLLNRVAEYATAAAAATAPPSAPPPGKKEVSGGGKITDEFTGKGSIGQVCQVIGAVVDVRFDEGLPPIMTALEVLDHSSRLVLEVAQHLGEGVVRTIAMDATEGVVRGWRVLNTGSPITVPVGRATLGRIINVIGEPIDDKGEINTEHYLPIHREAPAFVEQETAQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFVDNIFRFTQANSEVSALLGRIPSAVGYQPTLSTDLGALQERITTTKKGSITSVQAIYVPADDLTDPAPATTFAHLDATTVLSRQISELGIYPAVDPLDSTSRMLSPLILGADHYETARGVQKVLQNYKNLQDIIAILGMDELSEDDKLTVARARKIQRFLSQPFHVAEVFTGAPGKYVELKENITSFQGVLDGKYDDLPEQSFYMVGGIEEVIAKAEKIAKESAAS

SEQ ID NO: 28 - protein, 559 aa, Amborella trichopoda; identical to SEQ ID NO: 27

SEQ ID NO: 29 - protein, 551 aa, Solanum lycopersicum
MASRRFFSSMLRSSIRHSSTKSSLTNSIHRSSPVGHLLHRAVKYATAAAAKEAPAPQKKPTTIKGTGGKITDEYTGAGALGSVCQVIGAVVDVRFDEGLPPILTALEVLDHDIRVVLEVAQHMGENMVRTIAMDGTEGLVRGQRVLNTGSPIKVPVGRATLGRIINVIGEPIDERGDLKTEHYLPIHREAPSFVEQATEQQILVTGIKVVDLLAPYQRGGKIGLFGGAGVGKTVLIMELINNVAKAHGGFSVFAGVGERTREGNDLYREMIESGVIKLGEKQGESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKLTVARARKIQRFLSQPFHVAEVFTGAPGKYVELKESIQSFQGVLDGKYDDLSEQSFYLVGGIEEVIAKAEKIAKESAS

SEQ ID NO: 30 - protein, 558 aa, Cucumis sativus
MASRRLLSSLLRSSARRASSRSPFSNPNPRISPSTTSRASPRGYILSRVAEYATSAAATAPPSPPPPAKDAGTGGKITDEFTGAGSIGQVCQVIGAVVDVRFDEGLPPILTALEVLDHSIRLVLEVAQHLGENMVRTIAMDGTEGLVRGRRVLNTGSPITVPVGRATLGRIINVIGEPIDERGDLKTDHYLPIHREAPAFVDQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKMTVARARKIQRFLSQPFHVAEVFTGAPGKYVDLKESIGSFQGVLDGKYDDLPEQSFYMVGGIEEVIAKAEKIAKESAA

SEQ ID NO: 31 - protein, 559 aa, Glycine max
MASRRLVSSLIRSSLRRSQSKPSISASTSRLTSSNRASPHGYLLNRVAEYATAAAAATTPPSPPPPGKKELGGGGKITDEFTGKGAIGQVCQVIGAVVDVRFDEGLPPIMTALEVLDHSSRLVLEVAQHLGEGVVRTIAMDATEGVVRGWRVLNTGSPITVPVGRATLGRIINVIGEPIDAKGEINTEHYLPIHREAPAFVEQETAQQILVTGIKVVDLLAPYQRGGKIGLFGGAGVGKTVLIMELINNVAKAHGGFSVFAGVGERTREGNDLYREMIESGVIKLDDKQSESKCALVYGQMNEPPGARARVGLTGLTVAEHFRDAEGQDVLLFVDNIFRFTQANSEVSALLGRIPSAVGYQPTLSTDLGALQERITTTKKGSITSVQAIYVPADDLTDPAPATTFAHLDATTVLSRQISELGIYPAVDPLDSTSRMLSPLILGADHYETARGVQKVLQNYKNLQDIIAILGMDELSEDDKLTVARARKIQRFLSQPFHVAEVFTGAPGKYVELKENVASFQGVLDGKYDDLPEQSFYMVGGIEEVIAKAEKIAKESAAS

SEQ ID NO: 32 - protein, 553 aa, Brachypodium distachyon
MATRRALSSLLRSASRVRGASPSPLPRAAPHRPSPAGYLFNRAAAYATSAAAQSKPTTTPPPTSAGKTGPGKITDEFTGAGAVGQVCQVIGAVVDVRFDEGLPPILTALEVLDNSIRLVLEVAQHLGENVVRTIAMDGTEGLVRGQRVLNTGSPITVPVGRATLGRIINVIGEPIDERGDIKTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEEHYNTARGVQKVLQNYKNLQDIIAILGMDELSEDDKLTVARARKIQRFLSQPFHVAEVFTGAPGKYVELKESVQSFQGVLDGKYDDLSEQSFYMVGGIDEVIAKAEKIAKENA

SEQ ID NO: 33 - protein, 554 aa, Vitis vinifera
MASRKLLSSLLRTSVRRPISRSVLSNPRSPLPRPSPVGHLLARAANYASSASAAAPAASTPSAAKGAGPSGKITDEFTGAGAIGQVCQVIGAVVDVRFDEGLPPILTALEVLDNSIRLVLEVAQHLGENMVRTIAMDGTEGLVRGQRVLNTGSPITVPVGRATLGRIINVIGEPIDERGDIKTDHFLPIHREAPSFVDQATEQQILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEVFTGAPGKYVELKESITSFQGVLDGKYDDLSEQSFYMVGGIEEVIAKAEKIAKESAA

SEQ ID NO: 34 - protein, 551 aa, Brachypodium distachyon
MATRRAISSIVRSASRIRAASPSPLPRAPLHRPSPAGFLLNRAAAYATSAAAQAAPATPPPTSAKHTGGKITDEFTGAGSVGQVCQVIGAVVDVRFDEGLPPILTALEVLDNSIRLVLEVAQHLGENVVRTIAMDGTEGLVRGQRVLNTGSPITVPVGRATLGRIINVIGEPIDERGDIKTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEEHYNTARGVQKVLQNYKNLQDIIAILGMDELSEDDKLTVARARKIQRFLSQPFHVAEVFTGAPGKYVELKESVQSFQGVLDGKYDDLSEQSFYMVGGIDEVIAKAEKIAKENA

SEQ ID NO: 35 - protein, 1349 aa, Setaria italica
MATRRALSSILRSASRLRAASPSPVPRAPLHHRPSPAGFLLNRAASYASSAAAQAAPAPPPPSTGKTTGGGKITDEFTGAGAIGQVCQVIGAVVDVRFDEGLPPILTALEVLDNNIRLVLEVAQHLGENMVRTIAMDGTEGLVRGQRVLNTGSPITVPVGRATLGRIMNVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVKSFQGVLDGKYDDLPEQSFYMVGGIEEVIAKAEKIAKESLKISPNGSLLLPTVSFPNQRISKPQISSALLASSYSPYPISKHFKHSPPPSSHPIPFPFRSLLSPLRRRIARFPPRRQPAAPSPSRPLDEATPSGEEGATGVARPHAMSSAVKEQLQQMSTTCDSLLLELNVIWDEVGEPHSARDRMLLELEQECLEVYRRKVDMANRCRAQLRQAIAEAEAELAGICSAMGEPPIHVRQSNQKLQGLREELNAIVPYLEEMRKKKVERWDQFVDVIDQIKKVASEIRPADFVPFKFPVDQSDLSVRKLEELTKELQSLQKEKSDRLKQVMEHLNTLHSLCEVLGIDFKQTVHDVHPSLGEADGSKNLSNSTIERLASAVNGLREMKVQRMQKLQDLASSMLELWNLMDTPLEEQQMFQNVTCNIAASEHEITEPNTLSVDYLSYVESEVLRLEQLKASKMKDLVLKKKTELEEHRRRAHLIGEEGYAAEFSTEAIEAGAVDPALVLEQIEAHIATVKEEAFSRKDILEKVERWLNACEEEAWLEDYNKDDNRYNAGRGAHLTLKRAEKARILVNKIPGMVDVLTTKILAWQKEREKEFTYDGVRLLSMLEEYMIVRQEKELEKKRQRDQKKIQDQIKAEQEALYGSKPSPSKPQSTKKVPRNSMGGANRRLSLGGATMQAPKTDILHSKTARAAKKTEELGTLSPSSRGLDIAGLPIKKLTFNASTLRETETPRKPFAQIMPGNNVSSTPARPTFNDNTEEENKTPKTFNAALDLKTPMTVTAPMQLAMTPSVGNKVIATPVSLFQEKPEQPMLPEEIEYSFEERRLAVYLARQMA

SEQ ID NO: 36 - protein, 1128 aa, Cicer arietinum
MASRRIVSSLIRSSLRPSQSKSSITASTSRLSSQSRASPHAYILNRVTEYATSAAAAAAPPPSQAPPSKKVAGGGGKVTDEHTGKGAVGQICQVIGAVVDVRFEEGLPPILTALEVLDQPNRLVLEVAQHLGEGIVRTIAMDATEGVVRGWRVLNTGSPISVPVGRATLGRIMNVIGEPIDEKGDFKTEHYLPIHREAPDFVEQATEQEILVTGIKVVDLLAPYQRGGKIGLFGGAGVGKTVLIMELINNVAKAHGGFSVFAGVGERTREGNDLYREMIESGVIKLGDKQGESKCALVYGQMNEPPGARARVGLTGLTVAEHFRDAEGQDVLLFVDNIFRFTQANSEVSALLGRIPSAVGYQPTLSTDLGGLQERITTTKKGSITSVQAIYVPADDLTDPAPATTFAHLDATTVLSRQISELGIYPAVDPLDSTSRMLSPLILGDEHYQTARGVQQVLQNYKNLQDIIAILGMDELSEDDKLTVARARKIQRFLSQPFHVAEVFTGAPGKYVELKENTNSFQGVLNGKYDDLPEQAFYMVGGIDEVIAKADKIAKENATSYSLSPLLSLKMASRRLASSLIRSSLRRSSSKPSISASTSRLSSQSRASPYGYLLNRVADYATSAAAAAAPSTPPAKKAVPGGGKITDEFTGKGAIGHVCQVIGAVVDVRFEEGLPPILTALEILDHETRLVLEVAQHLGEGVVRTIAMDATEGVVRGWRVLNTGSPITVPVGRATLGRIMNVIGEPIDEKGELKTEHYLPIHREAPAFVEQATEQEILVTGIKVVDMLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFVDNIFRFTQANSEVSALLGRIPSAVGYQPTLSTDLGGLQERITTTKKGSITSVQAIYVPADDLTDPAPATTFAHLDATTVLSRQISELGIYPAVDPLDSTSRMLSPLILGDEHYQTARGVQKVLQNYKNLQDIIAILGMDELSEDDKLTVARARKIQRFLSQPFHVAEVFTGAPGKYVELKENITSFQGVLDGKYDDLSEQAFYMVGGIDEVIAKAEKIANESHASA

SEQ ID NO: 37 - protein, 552 aa, Oryza sativa
MATRRALSSLVRAASRLRGASPAPRPRGPLHRPSPSGYLFNRAAAYATAAAAKEAAPPAPATGKATGGGKITDEFTGAGAVGQVCQVIGAVVDVRFDEGLPPILTALEVLDHNIRLVLEVAQHLGENMVRTIAMDGTEGLVRGQRVLNTGSPITVPVGRATLGRIMNVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVNSFQGVLDGKYDDLPEQSFYMVGGIEEVIAKAEKIAKESAS

SEQ ID NO: 38 - protein, 557 aa, Oryza sativa
MATRRALTSVLRSASRLRAASPSPCPRRAPLHPHRRPSPAGFLLNRAAAAYASSAAAQAAPAPPPASGKATGGGKITDEFTGAGAVGQVCQVIGAVVDVRFDEGLPPILTALEVLDHNIRLVLEVAQHLGENMVRTIAMDGTEGLVRGQRVLNTGSPITVPVGRATLGRIMNVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVQSFQGVLDGKYDDLPEQSFYMVGGIDEVIAKAEKIAKESAS

SEQ ID NO: 39 - protein, 558 aa, Citrus sinensis
MASRRLLSSLLRSSVRRSPSKSSFSNSSVFSPASTRRASPYGHLLSRVSEYATSAAATAPPAQTPKSDVKKGGGGKITDEFTGKGSIGQVCQVIGAVVDVRFDEGLPPILTALEVVDHSVRLVLEVAQHMGEGVVRTIAMDGTEGLVRGQRVLNTGSPITVPVGRVTLGRIMNVIGEPIDEKGDLKTEHYLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQADSKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEVFTGAPGKYVELKESIASFQGVLDGKYDDLPEQSFYMVGGIEEVIAKAEKIAKESAA

SEQ ID NO: 40 - protein, 560 aa, Cucumis sativus
MASRRLLSSLLRSSARRSSSRSPISSPNPRIPLSNTSRASPYGYLLNRVAQYATSAAAATPPPSSPPSAKSDVGPSGKITDEFTGAGSIGQVCQVIGAVVDVRFDEGLPPILTALEVLDHSIRLVLEVAQHLGENMVRTIAMDGTEGLVRGRRVLNTGSPITVPVGRATLGRIINVIGEPIDEKGDLKTDHYLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQAESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKLTVARARKIQRFLSQPFHVAEVFTGAPGKYVELKESITSFQGVLDGKYDDLPEQSFYMIGGIEEVIAKAEKIARESAA

SEQ ID NO: 41 - protein, 1127 aa, Medicago truncatula
MASRRIVSSLIRSSLRPSLSKSSITTSTSRLSSSQSRASPYAYLLNRVTEYATAAAAAAPAPPQTPPAKKVPDGGGKITDEFTGKGAIGQVCQVIGAVVDVRFEEGLPPILTALEVLDHSTRLVLEVAQHMGEGVVRTIAMDATEGVVRGWRVLNTGSPISVPVGRCTLGRIMNVIGEPIDHKGDFKTEHFLPIHREAPLFVEQATEQQILVTGIKVVDLLAPYQRGGKIGLFGGAGVGKTVLIMELINNVAKAHGGFSVFAGVGERTREGNDLYREMIESGVIKLGDQQSESKCALVYGQMNEPPGARARVGLTGLTVAEHFRDAEGQDVLLFVDNIFRFTQANSEVSALLGRIPSAVGYQPTLSTDLGGLQERITTTKKGSITSVQAIYVPADDLTDPAPATTFAHLDATTVLSRQISELGIYPAVDPLDSTSRMLSPLILGDEHYETARGVQQVLQNYKNLQDIIAILGMDELSEDDKLTVARARKIQRFLSQPFHVAEVFTGAPGKYVDLKENCTSFKGVLDGKYDDLSEQAFYMVGGIDEVIAKAEKIAKENAHLHSLSLSSKMASRRLVSSLIRSSLRRSSSKPSITASTSRLTSQSRASPYGYLLNRVADYATAAAAAPAPSAPPAKKEVPGGGKITDEFTGKGAVGQVCQVIGAVVDVRFEEGLPPILTALEVLDHSSRLVLEVAQHLGEGVVRTIAMDATEGVVRGWRVLNTGSPISVPVGRATLGRIMNVIGEPIDHKGEFITEHYLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFVDNIFRFTQANSEVSALLGRIPSAVGYQPTLSTDLGGLQERITTTKKGSITSVQAIYVPADDLTDPAPATTFAHLDATTVLSRQISELGIYPAVDPLDSTSRMLSPLILGDEHYQTARGVQQVLQNYKNLQDIIAILGMDELSEDDKLTVARARKIQRFLSQPFHVAEVFTGAPGKYVELKENTQSFQGVLDGKYDDLSEQAFYMVGGIDEVIAKAEKIAKESAASSS

SEQ ID NO: 42 - protein, 557 aa, Vitis vinifera
MASRRLLSSLLRSSVRRYSAKSPAISPRTPSPYPSSRPSPKGFLLNRAVDYATSAAAAASPPPPPPAKGAGPSGKITDEFTGAGAIGSVCQVIGAVVDVRFDEGLPPILTALEVLDNSIRLVLEVAQHLGENMVRTIAMDGTEGLVRGQRVLNTGSPITVPVGRATLGRIINVIGEPIDERGEIKTDHFLPIHREAPSFVDQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEVFTGAPGKYVELKESITSFQGVLDGKYDDLSEQSFYMVGGIEEVIAKAEKIAKESAA

SEQ ID NO: 43 - protein, 551 aa, Solanum tuberosum
MASRRFFSSMLRSSIRHSSTKSSLTSSIYRSSPAGHLLHRAVKYATAAAAKEAPAPQKKPTTIKETGGKITDEYTGAGALGSVCQVIGAVVDVRFDEGLPPILTALEVLDHDIRVVLEVAQHMGENMVRTIAMDGTEGLVRGQRVLNTGSPIRVPVGRATLGRIINVIGEPIDERGDLKTEHYLPIHREAPSFVEQATEQQILVTGIKVVDLLAPYQRGGKIGLFGGAGVGKTVLIMELINNVAKAHGGFSVFAGVGERTREGNDLYREMIESGVIKLGEKQGESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKLTVARARKIQRFLSQPFHVAEVFTGAPGKYVELKESIQSFQGVLDGKYDDLSEQSFYLVGGIEEVIAKAEKIAKESAS

SEQ ID NO: 44 - protein, 561 aa, Ricinus communis
MASRRLLSSLLRSSTRRSVSKSPLSNSNPKLSTPSPISTRRASSYGYLLNRTSEYATSAAAAAAPPSTSEGVKGSGGGKITDDFTGKGAIGQVCQVIGAVVDVRFDEGLPPILTALEVLDHSIRLVLEVAQHLGGSMVRTIAMDGTEGLVRGQKVLNTGSPITVPVGRATLGRIINVIGEPIDEKGDLKTDHFLPIHREAPSFVDQATEQQILVTGIKVVDMLAPYQRGGKIGLFGGAGVGKTVLIMELINNVAKAHGGFSVFAGVGERTREGNDLYREMIESGVIKLGDKQAESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQKFLSQPFHVAEVFTGAPGKYVELKESVASFQGVLDGKYDDLPEQSFYMVGGIEEVIAKADKIAKELAA

SEQ ID NO: 45 - protein, 553 aa, Zea mays
MASRRVVSSLLRSASRLRAASPAAPRPRGAPHRPSPAGYLFNRAAAYASAAAAQAAPATPPPATGKTGGGKITDEFTGAGAIGQVCQVIGAVVDVRYDEGLPPILTALEVLDNDIRLVLEVAQHLGENMVRTIAMDGTEGLVRGQRVLNTGSPITVPVGRATLGRIINVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVKSFQGVLDGKYDDLPEQSFYMVGGIEEVIAKAEKIAKESAS

SEQ ID NO: 46 - protein, 551 aa, Fragaria vesca
MFSSRKLLSTLLRSSLRRTSSAAARPPLSRSSPITRSPGYLLSCAAHYSTSSAAAVSAPPKSGTPSTTGKITDEFTGKGAIGQVCQVIGAVVDVRFQDGLPPILTALEVLDNSIRLVLEVAQHLGENMVRTIAMDGTEGLVRGQRVLNTGSPITVPVGRATLGRIMNVIGEPIDHRGEINTDHSLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQSDSKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEVFTGAPGKYVELKESITSFQGVLDGKYDDLSEQSFYMVGGIEEVIAKAEKIAKENA

SEQ ID NO: 47 - protein, 558 aa, Populus trichocarpa
MASRRLLSSLLRSSSRRSISKSTLSSTNPKVSASSTRRASPYGYLLNRAAEYATSAAAASPPSQPPPVQSEGKKGKVIDEFTGKGSIGHVCQVIGAVVDVKFDEGLPPILTALEVQGHSIRLVLEVAQHLGESVVRTIAMDGTEGLVRGQPVLNTGSPITVPVGRATLGRIINVIGEAIDEKGDLKTEHYLPIHREAPSFVEQATEQQVLVTGIKVVDLLAPYQRGGKIGLFGGAGVGKTVLIMELINNVAKAHGGFSVFAGVGERTREGNDLYREMIESGVIKLGDQQAESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEEHYNTARGVQKVLQNYKNLQDIIAILGMDELSEDDKLTVARARKIQRFLSQPFHVAEVFTGAPGKYVELKEGVQSFQGVLDGKYDDLPEQSFYMVGGIEEVIAKAEKISKESAA

SEQ ID NO: 48 - protein, 553 aa, Setaria italica
MASRRVVSSLLRSASRIRAASPAAPRPRAPPHRPSPAGYLFNRAAAYASSAAAQAAPATPPPATGKTGGGKITDEFTGAGAIGQVCQVIGAVVDVRFDEGLPPILTALEVLDNNIRLVLEVAQHLGENMVRTIAMDGTEGLVRGQRVLNTGSPITVPVGRATLGRIMNVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVKSFQGVLDGKYDDLSEQSFYMVGGIEEVIAKAEKIAKESAS

SEQ ID NO: 49 - protein, 497 aa, Oryza brachyantha
PAPPPASGKATGGGKITDEFTGAGAVGQVCQVIGAVVDVRFDEGLPPILTALEVLDNNIRLVLEVAQHLGENMVRTIAMDGTEGLVRGQHVLNTGSPITVPVGRATLGRIMNVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVQSFQGVLDGKYDDLPEQSFYMVGGIDEVIAKAEKIAKESAS

SEQ ID NO: 50 - protein, 553 aa, Zea mays
MASRRVVSSLLRSASRLRAASPAAPRPRAPPHRPSPAGYLFNRAAAYASSAAAQAAPATPPPATGKTGGGKITDEFTGAGAIGQVCQVIGAVVDVRFDEGLPPILTALEVLDNNIRLVLEVAQHLGENMVRTIAMDGTEGLVRGQRVLNTGSPITVPVGRATLGRIINVIGEPIDEKGDIKTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLDDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVKSFQGVLDGKYDDLPEQSFYMVGGIEEVIAKAEKIAKESAS

SEQ ID NO: 51 - protein, 554 aa, Setaria italica
MASRRLLSSLLRSSSSALRRAGAPSPAAPRRASPAGLLLARFAASSAAQPAPPSAAPSSSPASAAGKGKGGKITDEFTGAGAVGQVCQVIGAVVDVRFDEGLPPILTALEVLDNNIRLVLEVAQHLGENMVRTIAMDGTEGLVRGQRVLNTGSPITVPVGRATLGRIMNVIGEPIDEKGDIKTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVKSFQGVLDGKYDDLPEQSFYMVGGIEEVIAKAEKIAKESAS

SEQ ID NO: 52 - protein, 559 aa, Populus trichocarpa
MASRRLLSSLLRSTSRRSLSKSTLSNSHPKLSTSSTRRASPYGYLLNRAAEYATSAAAASPSSQPPPAKSEGTKGKIIDEFTGKGSIGHVCQVIGAVVDVKFDEGLPPILTALEVQGHSIRLVLEVAQHLGESVVRTIAMDGTEGLIRGQPVLNTGSPITVPVGRATLGRIINVIGEAIDEKGDLKTEHYLPIHREAPAFVEQATEQQILVTGIKVVDLLAPYQRGGKIGLFGGAGVGKTVLIMELINNVAKAHGGFSVFAGVGERTREGNDLYREMIESGVIKLGDQQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKLTVARARKIQRFLSQPFHVAEVFTGAPGKYVELKEGVESFQGVLDGKYDDLPEQSFYMVGGIEEVIAKAEKISKESATA

SEQ ID NO: 53 - protein, 546 aa, Sorghum bicolor
MATRRALSSILRSASRLRSASPSPCPRAPLHHRPSPAGFILNRAAAYASSAAAQAAPAPPPPSTGKKTGGGKITDEFTGAGAIGQVCQVIGAVVDVRFDEGLPPILTALEVLDNNIRLVLEVAQHLGENMVRTIAMDGTEGLVRGQRVLNTGSPITVPVGRATLGRIINVIGEPIDERGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGYVLFSGVLDGKYDDLPEQSFYMVGGIEEVIAKAEKIAKENAS

SEQ ID NO: 54 - protein, 553 aa, Physcomitrella patens
MSGRKALAAALRAGSRRLSSSPQPPKGARSFSPNASNGRAVANAAAKGLSRSIFNSARAVTPALARGMSTDNKSTDFTGAGSVGSIATVIGAVVDVKFEKGLPPILTALEVQDHNLRVVLEVAQHLGENTVRTIAMETTDGLVRGQRVLNTGSPIMVPVGRATLGRIINVIGEAIDERGDISREHVLGIHREAPAFVDQGTEMEILETGIKVVDLLAPYQRGGKIGLFGGAGVGKTVLIMELINNIAKAHGGFSVFAGVGERTREGNDLYKEMIESGVIKLGDKQMESKCALVYGQMNEPPGARARVGLTGLTVAEHFRDAEGQDVLFFIDNIFRFTQANSEVSALLGRIPSAVGYQPTLATDLGGLQERITTTQKGSITSVQAIYVPADDLTDPAPATTFAHLDATTVLSRQISELGIYPAVDPLDSTSRMLSPRILGEEHYYCARDVQKVLQNYKNLQDIIAILGMDELSEDDKLTVARARKMQRFLSQPFHVAEVFTGSPGKYVDLKDSVSGFRGVLDGKYDDLPEQAFYMVGGIEEVSQKAEKLAKDMA

SEQ ID NO: 55 - protein, 486 aa, Physcomitrella patens
MSTDNKSTDFTGAGSVGSIATVIGAVVDVKFEKGLPPILTALEVQDHNLRVVLEVAQHLGENTVRTIAMETTDGLVRGQRVLNTGSPIMVPVGRATLGRIINVIGEAIDERGDISREHVLGIHREAPAFVDQGTEMEILETGIKVVDLLAPYQRGGKIGLFGGAGVGKTVLIMELINNIAKAHGGFSVFAGVGERTREGNDLYKEMIESGVIKLGDKQMESKCALVYGQMNEPPGARARVGLTGLTVAEHFRDAEGQDVLFFIDNIFRFTQANSEVSALLGRIPSAVGYQPTLATDLGGLQERITTTQKGSITSVQAIYVPADDLTDPAPATTFAHLDATTVLSRQISELGIYPAVDPLDSTSRMLSPRILGEEHYYCARDVQKVLQNYKNLQDIIAILGMDELSEDDKLTVARARKMQRFLSQPFHVAEVFTGSPGKYVDLKDSVAGFRGVLDGKYDDLPEQAFYMVGGIEEVSQKAEKLAKDMA

SEQ ID NO: 56 - protein, 425 aa, Oryza brachyantha
MVRTIAMDGTEGLVRGQRVLNTGSPITVPVGRATLGRIMNVIGEPIDEKGDITTNHFLPIHREAPAFVEQATEQQILVTGIKVVDLLAPYQRGGKIGLFGGAGVGKTVLIMELINNVAKAHGGFSVFAGVGERTREGNDLYREMIESGVIKLGDKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHVLGEDHYNTARGVQKVLQNYKNLQDIIAILGMDELSEDDKLTVARARKIQRFLSQPFHVAEVFTGAPGKYVELKESVNSFQGVLDGKYDDLPEQSFYMVGGIEEVIAKAEKIAKESAS

SEQ ID NO: 57 - protein, 580 aa, Volvox carteri
MLSGTSRFAATCLRQVAQGRNTALLNAVRAFAAEPAAAASADVGFVSQVIGPVVDVRFDGELPSILSSLEVQGHSVRLVMEVAQHMGDNTVRCIAMDSTDGLVRGQKVLNTGAPIKVPVGRGTLGRIMNVIGEPVDEQGPIDAAEVWSIHRDAPEFTEQSTEQEILVTGIKVVDLLAPYQRGGKIGLFGGAGVGKTVLIMELINNVAKAHGGFSVFAGVGERTREGNDLYREMIESGVIKLGDKRGESKCTLVYGQMNEPPGARARVALTGLTVAEYFRDVEGQDVLLFVDNIFRFTQANSEVSALLGRIPSAVGYQPTLATDLGGLQERITTTTKGSITSVQAVYVPADDLTDPAPATTFAHLDATTVLSRSIAELGIYPAVDPLDSTSRMLNPNIIGAEHYNVARGVQKVLQDYKNLQDIIAILGMDELSEEDKLTVARARKIQRFLSQPFQVAEVFTGTPGKYVDLKDTIAAFKGILEGKYDDLPEMAFYMVGGIHEVVDKADKLAKEVAARKDEGKKAKSAEALKDVPSLDKLVSEIKEEVVEADDGLEEDFKAEAISSENMVLNEKGDKVPLPKK

SEQ ID NO: 58 - protein, 574 aa, Chlamydomonas reinhardtii
MLSSVRLAALRAGKTNSVFQAVRAFAAEPAAAATTDAGFVSQVIGPVVDVRFDGELPSILSALEVQGHNVRLVLEVAQHMGDNTVRCVAMDSTDGLVRGQKVVNTGSPIKVPVGRGTLGRIMNVIGEPVDEQGPIECSEVWSIHREAPEFTEQSTEQEILVTGIKVVDLLAPYQRGGKIGLFGGAGVGKTVLIMELINNVAKAHGGFSVFAGVGERTREGNDLYREMIESGVIKLGDKRGESKCTLVYGQMNEPPGARARVALTGLTVAEYFRDVEGQDVLLFVDNIFRFTQANSEVSALLGRIPSAVGYQPTLATDLGGLQERITTTTKGSITSVQAVYVPADDLTDPAPATTFAHLDATTVLSRSIAELGIYPAVDPLDSTSRMLNPNIIGAEHYNIARGVQKVLQDYKNLQDIIAILGMDELSEEDKLTVARARKIQRFLSQPFQVAEVFTGTPGKYVDLKDTISAFTGILQGKYDDLPEMAFYMVGGIHEVVEKADKLAKDVAARKDESKKAKSSEALKDVPSLEKMAGEIKDEVIDADDSLEEDFKAEAISSENMVLNEKGEKVPLPKK

SEQ ID NO: 59 - DNA, 2027 bp, Arabidopsis thaliana
attcatttgataaccctagtagctccttttttctctatctcgctttctctgtttcaccacagtcatggcgagtcggcgaatcttatcatcgcttctccgttcttcttcgagtagatctacttctaaatcctccttgatcgggagccgaaacccgaggcttttatcccccggtcccgcgcatggagccgctccatgtgggactctccttggccgagtcgccgagtattcgacttcttctccggctaattcggctgcaccatcttctgctcctgctaaagatgagggaaagaagacctatgattacggtggcaaaggcgcgatcgggcgtgtgtgtcaggttattggtgccattgttgatgtgagattcgaagatcaggagggattgcctccgatcatgacatctctcgaggtacaggatcatccaacaaggttggtccttgaggtgtctcatcacttgggtcaaaatgtcgttaggaccattgctatggatggtactgagggtctcgtccgtggaaggaaggttctcaacactggtgctccaatcactgttcctgttggaagggccacccttggacgtatcatgaacgttcttggagaacctattgacgagagaggcgaaattaagaccgagcattacttacccattcacagagacgctccagctttggttgatttggccaccggtcaagagattctcgctactggtattaaggttgttgatctccttgctccttaccaaagaggaggaaagattgggctttttggcggtgctggtgttgggaaaacagtgctcattatggaactgattaacaatgttgccaaagctcatggtggtttctccgtgtttgctggtgtgggagaacggacccgtgaaggcaatgatttgtacagagaaatgattgagagtggtgtcatcaagctaggagagaaacagtctgagagcaaatgtgccctagtgtatggacaaatgaatgagcccccgggtgctcgtgcccgtgttggactgactggtttgactgttgctgagtatttccgtgatgctgaaggccaagatgtcttgcttttcattgacaacatcttccgtttcacccaggccaactctgaagtgtctgcgttgctcggtcgtatcccgtctgctgtggggtaccagccaactctggcttccgatcttggtgctcttcaagagcgaatcacaaccaccaagaaaggttcaattacctctgtccaagccatctatgtccctgctgatgatttgacagatcctgctcctgccacaacttttgctcacttggatgccacaactgtgctttcaagacagatttctgagcttggtatctaccctgctgtggatcctttggattcgacgtcccgtatgctctcgccccacattctaggtgaggagcactacaacacagctcgtggtgtgcagaaagtgttgcagaattacaagaatttgcaagatattatcgccattttgggaatggatgagctaagtgaagatgacaagctgactgttgcccgtgcccgtaagatccagagattcttgagtcagccattccatgttgctgaaatcttcactggtgcccctggaaaatatgtcgaccttaaagaaaacatcaacagtttccagggtttgttggatggcaagtacgatgatctttccgaacaatcgttttacatggttggaggtatcgatgaggtggttgcaaaggcagagaagatcgctaaagagtcagcagcttagaagctggatccatctcttgcatatgcttaatacctgtctgtttcttgtgacaataacgaaaagaaaaaaaccactgcctgtgagttacccacaccaagcggaatttgagttccgtatttatagtttctttcgaattccttgagagctacgagatgatgctgaacatacccttcattcccttgtactcagatttttggttcatttacttccttgacttgttgaaataaaggttaagcaagtcttgcagattatgatttcttgatctcagactttttctttcaga

SEQ ID NO: 60 - DNA, 2015 bp, Arabidopsis lyrata
attcatttgataaccctagtagcccctttttctctgtttcacagtcatggcgagtcggcgaatcttatcatcgctcctccgttcttcttccggcagatctacttctaaatcctccttgatcgggagccgaaatccaaggctttcatcaccaggtcccgctcatcgagccgcgccatgtgggactctccttggccgagtcgctgagtattcgacttcttctccggctaattcggctgcgccatcgcctgctcctgctaaggatgaggggaagaagacctacgattacggtggcaaaggcgcgatcgggcgtgtttgtcaggttattggtgccattgttgatgttagattcgaagatcaggagggattgcctccgatcatgacatctctcgaggtacaggatcaccccacaaggttggtccttgaggtgtctcatcacttgggtcaaaatgtcgttaggaccattgctatggatggtactgagggtctcgtccgtggaaggaaagttctcaatactggcgctccaatcactgttcctgttggaagggccacccttggacgtatcatgaatgttcttggcgaacccattgacgagagaggcgaaattaagaccgagcattacttacccattcacagagacgctccagctttggttgatttggccaccggtcaagaaattcttgctactggtattaaggttgttgatctccttgctccttaccaaagaggaggaaagattgggctttttggcggtgctggcgttgggaaaacagtgctcattatggaactgatcaacaatgttgccaaagctcatggtggtttctccgtgtttgctggtgtgggagaaaggacccgtgaaggcaatgacttgtacagagaaatgattgagagtggtgtcatcaagctaggcgagaaacagtccgagagcaaatgtgctctagtgtatggacaaatgaatgagcccccgggtgcccgtgctcgtgttggactgactggtttgactgttgccgagtatttccgtgatgccgaaggccaggatgtcttgcttttcattgacaacatcttccgtttcacccaggccaactctgaagtgtctgctttgctcggtcgtatcccgtctgctgtgggttaccagccaaccctggcttccgatcttggtgctcttcaagagcgaatcactaccaccaagaaaggttcaattacctcagtccaagccatctatgtccctgctgatgatttgacagatcctgctcctgccacaacttttgctcacttggacgccacaactgtgctgtcaagacagatttctgagcttggtatctaccctgctgtggatcctttggattcgacgtcccgtatgctctcccctcacattttgggtgaggagcactacaacacagctcgtggtgtgcaaaaagtgctacagaactacaagaatttacaagatattatcgccattttgggaatggatgagttaagtgaagatgacaagctgactgttgcccgtgcccgtaagatccagagattcctgagtcagccattccatgttgctgaaatcttcactggtgcccctggaaaatatgtcgaccttaaagaaaacatcaacagtttccagggtttgttggatggcaagtacgatgatctttccgaacaatcgttttacatggttggaggtatcgatgaggtggttgcaaaggcagagaagatcgctaaagagtcagcagcttagaagctggattaatctctttcatatgcttaatacttgtctgtttcttgtgacaataacgaaaaaaaaaaccactgcctgtgagttacctacccccaccaagcggatttttagttccgtatttatagtttctttgggttcggattccaattgagagctacgaaatgatgctgaacatatccttcattcccttgtactcagatttttggttcatttacttccttgacttgttgaaataaaggttaagcaagtcttgcagattatgatttcttgatctcaggatttttctttc

SEQ ID NO: 61 - DNA, 1737 bp, Arabidopsis lyrata
acacacacccactcatggcgtctcggagagttttatcatctcttctccgttcttcttccggcagatctgctgccaaactaggcaaccgtaaccccaggcttccttctccttcacccgcgcgtcacgccgctccatgtagctacctcctcggtcgcgtcgccgagtacgccacctcttcaccggcaagctcagctgcgccatcctctgctcctgctaaggatgaggggaagaagacctatgattacggtggcaaaggtgcgatcgggcgtgtttgccaagtcatcggtgccattgttgatgtgagattcgaggatcaggaaggattgcctccaatcatgacatctctcgaggtgcaggatcaccccacaaggctggtacttgaggtgtcccaccatttgggtcaaaatgtcgtcaggaccattgctatggatggtactgagggtcttgtccgtggaaggaaagttctcaacactggtgctccaatcactgtgcctgttggaagagctacccttggacgtatcatgaacgttcttggagaacccattgacgagagaggcgaaattaagaccgaacattacttacctattcacagagatgcaccggctttggttgatctagctactgggcaagagatcctggccactggtattaaggttgttgatcttcttgctccttaccaaagaggaggaaagattggtctctttggcggagctggtgttgggaaaactgtgcttattatggaactgatcaacaatgttgcaaaagctcatggtggtttctctgtgtttgctggtgtgggagaacgaacccgtgaaggcaatgacttgtacagagaaatgattgagagtggtgtcatcaagctaggcgagaagcagtccgagagcaaatgtgctctagtgtatggacaaatgaatgagcccccgggtgcccgtgctcgtgttggactgactggtttgactgttgccgagtatttccgtgatgctgaaggccaggatgtcttgcttttcattgacaacatcttccgtttcacccaggccaactctgaagtgtctgctttgctcggtcgtatcccgtctgctgtgggttaccagccaactctggcttctgatcttggtgctcttcaagagcgaatcacaaccaccaagaaaggttcaattacctcagtccaagccatctatgtccctgctgatgatttgacagatcctgctcctgccacaacatttgctcacttggacgccacaactgtgctttcaagacagatttctgagcttggtatctatcctgctgtggatcctttggattcgacgtcccgtatgctctcgccccacattcttggtgaggagcactacaacactgctcgtggtgtgcaaaaagtgctacagaactacaagaacttgcaagatattatcgccattttgggtatggatgagctaagtgaagatgacaagctgactgttgcccgtgcccgtaagatccagagattcttgagtcagccattccatgttgctgaaatctttactggtgcccctggaaaatatgtcgaccttaaagaaaacattaacagtttccagggtttgttggatggcaagtacgatgatctttccgaacaatcgttttacatggttggaggtatcgatgaggtggttgcaaaggcagagaagatcgctaaagagtcggcagcttaagggattatgcttaatacttctcttcttcttttgttgacaataacgaaaaaaa

SEQ ID NO: 62 - DNA, 4160 bp, Capsella rubella
tttaggaggagttaaaaggagagacgagaagacgagagaggagagaagacaacattttcatattgtaaccctagcgagagagcccctttctctcttaatctcgctttctctgtttcacagtcatggcgagtcggcgaatcttatcatcgcttctccgttcatctacgggcagatctacttctaaatcctctttaatcgggagccgaaaccccaggctttcatctccaggtcccgcgcatcgagccgctccttgtgcgacactccttggccgagtcgctgagtattctacatcttctccggctaattccgctgcgccatcctccgctccagctaaggatgagggcaagaagacttatgattacggtggcaaaggtgcgatcgggcgtgtttgtcaggtcattggtgccattgttgatgttagattcgaagatcaggagggattacctccgatcatgacatctctcgaggttcaggatcatcccacgaggctggtgcttgaggtgtctcatcacttgggtcaaaatgtcgtcaggaccattgctatggatggtactgagggtctcgtccgcggaaggaaggttctcaacactggcgctccgatcacagttcctgttggaagagctactcttggacgtatcatgaatgttcttggagagcctattgacgagagaggcgaaatcaagaccgaccattacctacctattcacagagatgccccagctttggttgatctagccactgggcaagagatcctggccactggtattaaggttgttgatcttcttgctccttaccaaagaggaggaaaaattggtctttttggtggtgctggtgttgggaaaactgtgcttatcatggagctgatcaataatgttgccaaagctcatggtggtttctctgtgtttgctggtgtgggagaacgaactcgtgaaggcaatgacttgtacagagaaatgattgagagtggtgtcattaagctaggcgagaagcagtctgagagcaaatgtgctcttgtgtacggacaaatgaatgaacccccgggtgctcgtgctcgtgttggactgaccggtttaactgttgccgagtatttccgtgatgccgaaggccaagatgtcttgcttttcattgacaacattttccgtttcactcaggctaactctgaagtgtctgcgttgcttggacgtatcccatctgctgtgggttaccagccaactctggcttctgatcttggtgctcttcaagagcgaatcacaaccaccaagaaagggtctatcacctcagtccaagccatctatgtccctgctgatgatttgacagatcctgctcctgccacaacttttgctcacttggacgccacaactgtgttatcaagacagatttctgagcttggtatctatcctgctgtggatcctttggactcaacatcccgtatgctctcgcctcacattttgggtgaggagcactacaacactgctcgtggtgtgcagaaggtgctacagaactacaagaatttacaagatattattgccattttgggaatggatgagctaagtgaagatgacaagctgactgttgcccgggcccgtaagatccagagattcttgagtcagcctttccacgttgctgagatcttcactggtgcccccggaaaatacgtcgatctaaaggaaaacatcaacagtttccagggtctactcgacggtaagtacgatgatcttcctgaacaatcattttacatggttggtggcatcgatgaggttgttgcaaaagcagagaagatctccaaggagtcagcagcttaagagattaatttgtctctctcgtgcttaatactttcttcttttttctgacaataacgaaaaaaaaagccaatgcctgtgactctcccaccaaacggaatctgattccgtatttatagtttcttttggttcaaattccatgagagctggcaagatggtgctgaacagatccattcactgttactcaaatttttgtttattttgtttccattacttaatgaaataaagattaagtcttgcagattctaattgccatgtatgagaatgttctttattgttcttagtatctttgtaagcttagcacaagtcttgctatgatgttcatcgcaaagcaggatttttaatcatctcttcagaagctaacgtcccatttttgaaaacgtatcactatattgaaataatgtaaatgatctaacaacatgatgtctcattacaacttcttccatagccttcttttatgctctcttatatacacaaccaaacatgtatgcatcagaagcaattgagaccacaaaacaacactctccattatattttagaaactcaagctaccttctttcaacaatgctttataatctctgggcacatccacccgatcgatcacatccaacactttttccttgtcgataacaaaatcaagaagcgggaaacccgaaggatacctcttcaccgcagctgatttgcttcttctcactccacggaaatactcactgcttttgctcagagggtcacattttcttgcccgagaatattcataatcaaaatcttgactcttgctaacataaggagttctctttcgtttgcttgcctcttgcaacatattttgctcctcaactgttactaatgggacactgagtggttcagatatcaactgctggttcatagtattcaatggagacttgatagaagaaagtcgaggatctcctcgagcatcgctctttgcttttgtaaccaaatgttgtaaccaaattatttgtccaagaatgtagatctctgtcttttctttactagcgtgatagagtgtttcaatacgtaatatatctccaccactaggcttactagtgaaatcagtcctgtaagttaaaaaacgaaaccattggcggaaaagcacaaaaaaaaatgcaggagaaatagaaggtgcaggtctcatttcatccatcactcaccctgtatttgcccactccccaacccaaccaaaaccatgatgtgctctgcgaggaatccattcaacaatttagccattacaaaggagaatttatactccgagctgaacatgtaaagttggcgttatgaaagaagggtcagatttcttacttggttgtgttgcctgcaactgggacaagccaatgcagtgtcctctccatctcgtccttgatttgtgtaaccgaaagctgttcataaaaatcataaaccataacaccttgtgttcagtgtgagtagccaaagagccacgttatttcctcagttttgtacacccgtgactgtaaaacagatgacattacctccttatcgacattgaaagattttatcttggaacgtagagccaattttataccaggtggcaagctttggtatagagaatcccttgcatttgaagttatagagcttgcacgagcgacctatacccatcaaaacatcacatcctatatgattgcgagaatacacatagaccaaaccaaatgaagcaatcctacatttgtgcactcacaagtgtatcaatctgcacaattatgtttgcatagtgtaaagcaagaccagcaggtcccaatcttttatcataatctgcagcacctttccttgatggttggtcatctaggcaaagaaaatgaatttgatattatagttaaatttttgtgtgaaaacttaattgtgaggatcaattaactagagaaatcacagtatgtactaaaatagagcagtggacgactgaaaattcaaaagctttgatacatgctcagtcgcacatggtgaaagtacaaaaaggagaacttacataccagcaccaccgaaaatattatgaatctctagcaacaagaaatgtacaatatcaactagtttctccatcacctgaaatacaaattagcaggtaagccaaaaaaacatcatcataaaatctatttctgtatgcaagattttcctgacaatagatttagtttggctgaatttagttggtgaagtataatgtttgaagtgattgaaacgatgcattgacaccatacagctgaaggatttgatgatgttaaatgataaagctgtacacacctcttcaaaacctctggaccacaatgactttttctttaagcttttcactacctttctctgagctttaagttcagtttttaaaattgcaaggccatcgcc

SEQ ID NO: 63 - DNA, 2007 bp, Arabidopsis thaliana
gcatttcctcttccacacacccactcatggcgtctcgaagagttttatcatctcttctccgttcatcttccggtagatctgccgccaaattagtcaaccgaaaccccaggcttccttctccttcacccgcgcgtcacgccgctccatgtagctacctcctcggccgcgtcgccgagtatgcgacctcttcaccggcaagctctgctgcgccatcctctgctcctgctaaggatgaggggaaaaagacctatgattacggtggcaaaggtgcgatcgggcgtgtttgtcaggtcatcggtgccattgtcgatgtgagattcgaggatcaggaaggattgcctccgatcatgacatctcttgaggttcaggatcatcccacaaggctggtgcttgaggtgtctcatcatttgggtcaaaatgtcgttaggaccattgctatggatggtactgagggtcttgtccgtggaaggaaggttctcaacactggtgctccaatcactgtacctgttggaagagctacccttggccgtatcatgaatgtgcttggagaacccattgacgagagaggcgaaatcaagaccgagcattacttacctattcacagagatgctccggctttggttgatctagccactgggcaagagatcctggccactggtattaaggttgttgatcttcttgctccttaccaaagaggaggaaagattggtctctttggcggtgctggtgttgggaaaactgtgcttattatggagctgatcaacaatgttgccaaagctcatggtgggttctctgtgtttgctggtgtgggggaacgaacccgtgagggcaatgacttgtacagagaaatgattgagagtggtgtcatcaagctaggcgagaagcagtctgagagcaaatgtgccctagtgtatggacaaatgaatgagcccccgggtgctcgtgcccgtgttggactgactggtttgactgtggctgagtatttccgtgatgctgaaggccaagatgtcttgcttttcattgacaacattttccgtttcactcaggccaactctgaagtgtctgctttgctcggtcgtattccgtctgctgtgggttaccagccaactctggcttctgatcttggtgctctgcaagagcgaatcacaaccaccaagaaagggtctatcacctcagtccaagccatctatgtccctgctgatgatttgacggatcctgctcctgccacaacttttgctcacttggacgccacaaccgtgctgtcaagacagatttctgagctcggtatctatcctgctgtggatcctttggattcgacatcccgtatgctctccccccacattctgggtgaggagcactacaacactgctcgtggtgtgcagaaagtgttgcagaactacaaaaatttgcaagatattattgccattttgggaatggatgagctaagtgaagatgacaagctgactgttgcccgtgcccgtaagatccagagattcttgagtcagccattccatgttgctgagatcttcacgggtgcccctggaaaatacgttgatcttaaggaaaacatcaacagtttccagggtctactcgacggtaagtacgatgatctttccgaacaatcgttttacatggttggaggtatcgatgaggtggttgcaaaggcagagaagatcgctaaagagtcagcagcttaaggaattgattcatctctttcatatgcataatacttctcttcttcttttgttgacaataacgaaaaaacaacaagaccaatgctttaggttgtctgctgaggcattgtgatcattgtgagtgtgagagatagtacggagaaaacagattcattcgttgaagttttgacttggaataatggagacaggtgttgatgttttcaatctctccacatttgtattcaagcttttgtattaaggaaagtatcgtgtttttacaggattcgatgttagaaatgaatttgcacttttattagtcttcttcttctgtatt

SEQ ID NO: 64 - DNA, 1901 bp, Arabidopsis lyrata
tctctctcaaacacacacccactcatggcgtctcggagagtcttatcatcgcttctccgttcatcttccggcagatctgctgccaaattaggcaaccgtaaccccaggcttccttctccttcacccgctcgtcacgccgctccatgtagctacctcctcggccgtgtcgccgagtacgcgacctcttcaccggcaagctcagctgcgccatcttctgctcctgctaaggatgagggaaagaagacctatgattacggtggtaaaggtgcgatcgggcgtgtttgccaggtcatcggtgccattgtcgatgtgagattcgaggatcaggaaggattgcctccgatcatgacatccctcgaggtgcaggatcaccctacaaggctggtgcttgaggtgtctcaccatttgggtcaaaatgtcgtcaggaccattgctatggatggtactgagggtcttgtccgtggaaggaaggttctcaacactggtgctccgattactgtacctgttggaagagctactcttggacgtatcatgaatgttcttggagaacccatcgacgagagaggcgaaattaagaccgaacattacttacctattcacagagatgctccggctttggttgatctagctactgggcaagagatcctggccactggtattaaggttgttgatcttcttgctccttaccaaagaggaggaaagattggtctctttggcggtgctggtgttgggaaaactgtgcttattatggagctgatcaacaatgttgcaaaagctcatggtggtttctctgtgtttgctggtgtgggagaacgaacccgtgaaggcaatgacttgtacagagaaatgattgagagtggtgtcatcaagctaggcgagaagcagtctgagagcaagtgtgctctagtgtacggacaaatgaatgagcccccgggtgcccgtgcccgtgttggactgactggtttgactgttgccgagtatttccgtgatgccgaaggccaagacgtcttgcttttcattgacaacattttccgtttcactcaggccaactctgaagtgtctgctttgcttggtcgtatcccgtctgctgtgggctaccagccaactctggcttccgatcttggtgctcttcaagagagaattactaccaccaagaaagggtctatcacctcagtccaagccatctatgtacctgctgatgatttgactgatcctgctcctgctacaacttttgctcacttggacgccacaactgtgctttcaagacagatttctgagcttggtatctaccctgctgtggatcctttggattcaacatcccgtatgctgtcacctcacattctgggagaggagcactacaacacggctcgtggcgtgcagaaagtgctacagaactacaagaacttgcaagatattattgccattttgggaatggatgagctaagtgaagatgacaagttgactgttgcccgtgcccgtaagatccagagattcttgagtcagccattccacgttgctgagatcttcactggtgcccctggaaaatatgtcgaccttaaagaaaacatcaacagtttccagggtttgttggatggcaagtacgatgatctttctgaacaatcgttttacatggttggaggtatcgatgaggtggttgcaaaggcagagaagatcgctaaagagtcagcagcttaagggattgattcagctctttcatgcttaatacttctcttatttttctgacaataacgaaaaaaaaaaaacaatgcctgcgactccccgaccaaacggaatctgagttccgtaattatagtttcttttggttcaaattccatgagagctggcaagatgatactgaacagatccattcactgttactcaaatttttggttgttttgctt

SEQ ID NO: 65 - DNA, 1976 bp, Arabidopsis thaliana
ctctctctctcaaacacacacccactcatggcgtctcggagagtcttatcatctcttctccgttcatcttccggtagatctgccgccaaattaggcaaccgtaaccccaggcttccttctccttcacccgcgcgtcccgccgctccatgtagctacctcctcggccgcgtcgccgagtacgcgacctcttcacctgcaagctcagctgcgccatcttctgctcctgctaaggatgaggggaagaagacctatgattacggtggcaaaggtgcgatcgggcgtgtttgccaggtcattggtgccattgtcgatgtgagatttgaggatcaggaaggattgccaccaatcatgacatctctcgaggtgcaggatcaccccacaaggctagtgcttgaggtgtctcatcacttgggtcagaatgtcgtcaggaccattgctatggatggtactgagggtctcgttcgtggaaggaaagtcctcaacactggcgctccaatcactgtacctgtcggaagggctactcttggacgtatcatgaatgttcttggagaacctattgacgaaagaggcgaaattaagaccgaacattacttacctattcacagagatgcaccggctttggttgatctagctactgggcaagagatcctggccactggtattaaggttgttgatcttcttgctccttaccaaagaggaggaaagattggtctctttggcggtgctggtgttgggaaaactgtgcttattatggagctgatcaacaatgttgccaaagctcatggtggtttctctgtgtttgctggtgtgggagaacgaacccgtgaaggcaatgacttgtacagagaaatgattgagagtggtgtcatcaagctaggcgaaaagcagtctgagagcaagtgtgctctagtgtacggacaaatgaatgagcccccgggtgcccgtgcccgtgttggactgactggtttgactgttgccgagtatttccgtgatgctgaaggccaagacgttttgcttttcattgacaacattttccgtttcactcaggccaactctgaagtgtctgctttactcggtcgtatcccgtctgctgtgggataccagccaactctggcttctgatcttggtgctcttcaagagcgaattactaccaccaagaaagggtctatcacctcagtccaagccatctatgtacctgctgatgatttgactgatcctgctcctgctacaacttttgctcacttggacgccacaactgtgctctcaagacagatttctgagcttggtatctaccctgctgtggatcctttggattcaacatcccgtatgctgtcacctcacattctgggagaggagcattacaacacggctcgtggcgtgcagaaagttctacagaactacaagaacttgcaagatattattgcaattttgggtatggatgagctaagtgaagatgacaagttgactgttgcccgtgcccgtaagatccagagattcttgagtcagccgttccacgttgctgagatcttcactggtgcccctggaaaatatgtcgaccttaaagaaaatatcaacagtttccagggtttgttggatggcaagtacgatgatctttccgaacaatcgttttacatggttggaggtatcgatgaggtggttgcaaaggcagagaagatcgctaaagagtcagcagcttaagagattgattcagatctttcatgcttaatacttctcttttttttcctgacaataacgaaaaaaaaaaaccaatgcgtgcgactctccgaccaaacggaatctgagttccgtaattatagtttcttttggttcaaattccatgagagctggcaagatgatactgaacagatccattcactgttaatcaaatttttggttgttttgcttcctttgttgaaataaagatgaaaaatcttgcagattctaattgcaatatgtgtttaagatttcttgatgcc

SEQ ID NO: 66 - DNA, 2649 bp, Capsella rubella
atgttcccaacatttcaccaccaaaactatatgtctagcatgatctcaattctcaactttgatctcccaactttcttatttatattgtggatccccaagggccaagggtcactcttcttgattcggcaatcagcatatgttgttaccctagcatttcctctctgtctcttttctatctctctcacacacctactcatggcgtctcggagagtcttatcatcgcttctccgttcatcttccggtagatctgctgctaaattaggcaaccgtaaccccaggcttccttctccttcacccgcgcgtcacgctgctccatgtagttatctcctcggccgcgtcgccgagtacgccaccgcttcaccggcaagctcagctgcgccatcttctgctcctgctaaggatgagggcaagaagacctatgattacggtggtaagggtgcgatcgggcgtgtttgccaggtcattggtgccattgtcgatgtgagattcgaggatcaggagggattgcctccgatcatgacatctctcgaggtccaggatcaccccacgaggctggtccttgaggtttctcatcacttgggtcaaaatgtcgtcaggaccattgctatggatggtactgagggtctcgtccgtggaaggaaggttctcaacactggcgctccaatcactgttcctgttggaagagctactcttggacgtatcatgaatgttcttggagagcccattgacgagagaggcgaaattaagaccgaccattacctacctattcacagagatgctccagctttggttgacctagccactgggcaagagatcctggccactggtattaaggttgttgatctccttgctccttaccaaagaggaggaaaaattggtctctttggcggtgctggtgttgggaaaactgtgcttatcatggagctgatcaataatgttgccaaagctcatggtggtttctctgtgtttgctggtgtgggagaacgaactcgtgagggcaatgacttgtacagagaaatgattgagagtggtgtcattaagctaggcgagaagcagtctgagagcaaatgtgctcttgtgtacggacaaatgaatgaacccccgggtgctcgtgctcgtgttggactgaccggattgactgttgccgaatatttccgtgatgcggaaggccaagatgtcttgcttttcattgacaacattttccgatttactcaggccaactctgaagtgtctgctttgcttggacgtatcccatctgctgtgggttaccagccaactctggcttctgatcttggtgctcttcaagagcgaatcacaaccaccaagaaagggtctatcacctcagtccaagccatctatgtccctgctgatgatttgacagatcctgctcctgccacaacttttgctcacttggacgccacaactgtgttatcaagacagatttctgagcttggtatctatcctgctgtggatcctttggattcgacatcccgtatgctctcgcctcacattttgggtgaggagcactacaacactgctcgtggtgtgcagaaggtgttacagaactacaagaatttacaagatattattgccattttgggaatggatgagctaagtgaagatgacaagctgactgttgcccgtgcccgtaagatccagagattcttgagtcagcctttccacgttgctgagatcttcactggtgcccctggaaaatacgtcgatcttaaggaaaacatcaacagtttccagggtctacttgacggtaagtacgatgatcttcctgaacaatcattttacatggttggtggcatcgacgaggtggtggctaaggcggagaagatctccaaggaatcagcagcttaagggattgattcatctctttcatgtgcttaatacttctcttcttcttttgttgacaataacgaaaaaaaaacaagagcaatgcttccggctgtttctgctgatgcattgctaccattgtgagtgtgagagatatagtacggagaaattagattcattcgtcagagtttttgacttcgaataaaggagacaagtgtccatgttttcaatctggccacatttgtattgaagttttcgtaaggaaagtgcttttacaggattcaatgttttgctgttcaaagtacgactcagattcatttgaaaagaaatgttgaatttgtctcttctttatatattattttattcttacaagtcggagtcgaaaccgcaaggtttctctctttagaatgaattccgatgtacaatctccacactcgagtaatgaatatgagtataagacagaataaattaacaaaacatatgtatatgaagttatgaactaacactgagcagaaggagtcttcacaaatgttcgagaactgctgctgtatgattgaagccacaagatatatgcactgctcttatattctttcccaagtccaccatcgctggtcttgcatagtctacatcactaccatgcccctacatatttaacataccatattatttattctctttgatgccagatttcttggaaaaacatgaaattatatcgcattcacataagaaattaatctttcaggaactgatcaataagaaagatattgaaacataccagttgtccaccagaggaatgtccatcaacag

SEQ ID NO: 67 - DNA, 1677 bp, Capsella rubella
atggcgtctcggagagttttatcatcgctgctccgttcatcttccggtagatctgctgccaaattaggaaatcgaaaccctaggcttccttctccttcaccagctcgtcacgccgctccatgtagctaccttctcggccgcgtcgccgagtacgcgaccgcttctccggcaagctcagctaaggatgcgccatcgtctgctcctgctaaggatgagggcaagaagacctatgattacggtggtaaaggtgcgatcgggcgtgtttgccaggtcattggtgccattgtcgatgtgagattcgaggatcaggaaggattgcctccaatcatgacatctctcgaggtccaggatcaccccaccaggcttgtccttgaggtttctcatcatttgggtcaaaatgtcgtcaggaccattgctatggatggtactgagggtctcgtccgtggaaggaaggttctcaacactggcgctccaattactgtccctgttggaagagctactctaggacgtattatgaatgttcttggagagcccattgacgagagaggcgaaattaagaccgaccattacctacctattcacagagatgctccagctttggttgatctagccactgggcaagagatcctggccactggtattaaggttgttgatctccttgctccttaccaaagaggaggaaagattggtctctttggcggtgctggtgttggaaaaactgtgcttatcatggagctgatcaataatgttgccaaagctcatggtggtttctctgtgtttgctggggtgggagaacgaactcgtgagggcaatgacttgtacagagaaatgattgagagtggtgtcatcaagctaggcgagaagcagtctgagagcaaatgtgctcttgtgtacggacaaatgaatgagcccccgggtgctcgtgcccgtgttggactgactggtttgactgttgccgagtatttccgtgatgctgaaggccaagatgtcttgcttttcattgacaacattttccgtttcactcaggccaactctgaagtgtctgctttgcttggacgtatcccatctgctgtgggttaccagccaactctggcttctgatctcggtgctcttcaagagcgaattacaaccaccaagaaagggtctatcacctcagtccaagccatctatgtccctgctgatgatttgacagatcctgctcctgccacaacttttgctcacttggacgccacaaccgtgttgtcaagacagatttctgagctaggtatctatcctgctgtggatcctttggattcgacatcccgtatgctctcgcctcacatattgggtgaggagcactacaacacagctcgtggtgtgcagaaggtgctacagaactacaagaatttacaagatattattgccattttgggaatggatgagctaagtgaagatgacaagttgactgttgcccgtgcccgtaagatccagagattcttgagtcagcctttccacgttgctgaaatcttcactggtgcccctggaaaatacgtcgatctcaaggaaaacatcaacagtttccagggtctactcgacggtaagtatgatgatcttcctgaacaatcattttacatggttggtggcatcgacgaggtggtggcaaaggcagagaagatctccaaggaatcagcagcttaa

SEQ ID NO: 68 - DNA, 2316 bp, Eutrema salsugineum
atggagcttagattcaggttttgttgttaccctagcatttcctctctctcacacacaacaaacccactcatggcgtctcggagagtcttatcatcgctgctccgttcatcttccggcagatctgctgccaaattcgggagccggagccccaggattccttctccttcccctgctcgttgtgtttctcctttcggagacctcctcggccgcgtcgccgaatattcgacttcttcaccggcaaactcagctgctccatcgtctgcccctgccaaggatgaggggaagaagacctatgattacggtggtaaaggtgcgatcggaaaggtttgccaggtcattggtgccattgttgatgtaagattcgaagatcaggagggattgcctccgatcatgacatctctcgaggtgcaagaccatcccacgaggctggtgctcgaggtgtctcatcacttgggtcagaatgtcgtcagaaccattgcgatggatggtaccgagggtctcgtccgtggaaggcgcgttctcaacactggcgctccaatcactgtccctgttggaagagctacccttggacgtatcatgaatgttcttggtgaacccatcgatgagagaggcgaaattaaaaccgaccattacttacctattcacagagatgctccggctttagttgatctagccactgggcaagagatcctggcaactggtattaaggttgttgatctgcttgctccttaccaaagaggaggaaagattgggctctttggcggtgctggtgttgggaaaactgtgctcattatggagctgatcaacaatgtcgccaaggctcatggtggtttctctgtgtttgctggggtgggagaacgaacccgtgaaggcaatgacctgtacagagaaatgattgagagtggtgtcatcaagctaggcgagaagcagtcagagagcaaatgcgctcttgtgtatggacaaatgaatgagcccccgggtgctcgtgcccgtgttggacttactggattgactgtcgctgagtatttccgtgatgctgaaggccaagatgtcttgctttttatcgacaacattttccgtttcactcaggccaactcagaagtgtctgctttacttggacgtatcccgtctgctgtcggttaccagccaactttggcttccgatcttggtgctcttcaggagcgaatcacaaccactaagaaaggttctatcacctcagtccaagccatctatgtccctgctgatgatttgactgatcctgctccagccacgacttttgctcacttggacgccacaaccgtgctctcgagacagatatctgagcttggtatctatcctgctgtggatcctctggattcgacatcccgtatgctctcacctcacattctgggtgaggatcactacaacacggctcgtggggtgcagaaagtcctacagaactacaagaacttgcaagatattatcgccattttgggaatggatgagcttagtgaagatgacaagttgactgttgcccgtgcccgtaagatccagagattcttgagtcagccattccatgttgctgagatcttcacgggtgccccaggaaaatacgttgatcttaaggaaaacatcaacagtttccagggtttgctcgatggtaagtacgatgatcttcctgaacaatcgttttacatggttggtgggattgacgaggtggttgcaaaggcagagaagatctccaaggagtcagcagcttaagtgcttgatccatctttcttgggtgcttaatacttctcatcttcttcttttgttgacaataacgaaaacaaaaccaatgctttaggctgagctgctaatgcattgcgattcccatcattgtgagtgagagatatagtacggagaaaaaatgttcattagttggagattttgacttccaataaagaagacaaagtgttgatgttttccattttctccaaattgtattaagttatcgtattcttaagaaaagttattttgacagctttcactgttattctttttcaagtaagaaaccattgacaagaaatgtttttaaaaaaattagtgttgaatctgcacttcacatttaatttttcttgtccttctgtatttagggaatcttttatttttacaaatgcagcaccattagtttcttcctatgagttgattccgatgtacaacctcctcacttaggtataagaaagaataaacaaacgtaaaacatactgtatatagtcttcaataatgttcgagaaccgctcctgtatgattgaagccacaagatatatgcactgctcttacattctttcccaagtt

SEQ ID NO: 69 - DNA, 3772 bp, Cicer arietinum
tctctctctctctttctcttttctctcccgctcaagatggcttcacggagaatcgtttcatctctgattcgatcttccctccgtccatctcaatccaaatcatccatcactgcttcaacatcaagactctcttctcaatcacgcgcttctccacacgcttacattctcaaccgtgtaactgaatatgccacgtcagccgctgctgctgctgcacctcctccttctcaagcgcctccgtccaagaaagttgctggcggtggtggaaaggttactgatgagcacaccggtaagggtgcggttggacagatttgtcaggtgattggtgctgttgttgatgtgagattcgaagaaggtttgcctcccattttgaccgcgcttgaggttttggatcagcctaatcgcttggtgttggaggtggcacagcatttgggtgagggaattgttagaactattgctatggatgctactgaaggggttgttagaggatggcgtgtcctcaacaccggctctcctatcagcgttcctgttggcagagctactcttggacgtatcatgaatgttatcggagaacctatcgacgagaaaggcgatttcaaaaccgagcattatttgccaattcacagagaagctcctgattttgttgaacaagcaacagaacaagagattcttgttactggaatcaaggttgttgacttgcttgcaccataccaaagaggaggaaagattgggttgtttggtggtgctggtgtaggaaaaaccgtgcttattatggaacttattaataatgttgcaaaggctcacggtggtttctctgtttttgctggtgttggagaaagaacccgagagggtaatgacttgtacagagaaatgattgagagtggtgtcattaagctaggtgataagcagggtgaaagcaaatgtgctcttgtgtatggtcaaatgaatgagccacctggtgcacgtgcccgtgttggtcttaccggacttactgttgctgaacacttccgtgatgctgaaggacaagatgtgcttcttttcgttgacaacattttccgtttcactcaagcaaactcagaggtgtctgctttgcttggtcgcatcccatctgccgttggttaccaaccaacattgtctaccgatcttggaggtcttcaagagcgtattacaaccacaaagaagggttcaattacctctgtccaagctatctatgtgcctgctgatgacttgacagatcctgctcctgctaccacctttgctcacttggacgctacaacagtgttgtcaagacagatatctgagcttggtatctatcctgctgttgatccattggattcaacatctcgcatgctttctccacttattttgggtgatgaacactaccaaactgctcgtggtgtacaacaagtacttcaaaactacaagaatcttcaagatatcattgccattttgggaatggacgagcttagtgaagatgataagttgacggttgcacgtgcccgtaaaattcaacgattcttgagccagcctttccatgtggcagaagttttcactggtgccccagggaaatatgttgagttgaaggagaacactaatagtttccagggtgtgttgaatggcaaatatgatgacctcccagagcaagcattttacatggttggtggtattgatgaagtcattgcaaaggcagataagattgctaaggaaaatgccacgtcttactctctctctcctcttctctctctgaagatggcttcacgcagactcgcatcgtctctgattcgatcttctctccgtcgatcttcttccaaaccctccatttccgcctcaacatcaaggctctcttctcaatcacgtgcttctccttatggctaccttctaaaccgtgtcgctgattacgccacttccgccgcagctgctgcagctccttctacacctcctgcgaagaaggcggtgccaggaggtggaaaaatcaccgatgagttcactggtaaaggtgcgatcgggcatgtttgtcaggttattggtgccgtcgtcgatgtcagattcgaagagggtttgcctccgatcttgaccgcacttgagattttggatcatgagacacgattggtgttggaggttgcacagcatttgggtgaaggtgttgtcagaactattgctatggatgctactgaaggtgtcgttagaggatggcgcgtgctcaacactggttccccaatcactgttcctgttggtagggctacccttggtcgtatcatgaatgttattggagagcctattgatgagaagggtgaactaaaaaccgagcattatttgcccattcacagagaagcacctgcttttgttgagcaagcaactgaacaagagattcttgttactggtatcaaggttgttgacatgcttgcaccataccaaagaggaggaaagattggattgtttggtggtgccggtgtaggaaaaactgtgcttattatggaacttatcaacaatgttgctaaggctcatggtgggttctccgtgtttgctggtgttggagaacgaacacgagagggtaatgacttgtacagagaaatgattgagagtggtgtcattaagctaggcgataaacagagtgaaagcaaatgtgctctagtgtatggtcaaatgaacgaacccccgggtgctcgtgcccgtgttggtctaactggacttactgttgctgaacacttccgtgatgctgaaggacaggatgtgcttctttttgttgacaacattttccgctttacccaagcaaactcagaggtgtctgctttacttggtcgtatcccatctgctgttggttaccaaccaacattgtctaccgatcttggaggtcttcaagagcgtattacaaccacaaagaagggttcaattacctctgtccaagctatctatgtgcctgctgatgatttgacagatcctgctcctgccaccacctttgctcacttggatgccacaacagtgttgtcacgacagatctctgagcttggtatctatcctgctgtcgacccattggactccacatctcgtatgctttccccacttattttgggtgatgaacactatcaaactgctcgtggtgtacagaaagtacttcaaaactataagaatcttcaagatatcattgccattttgggaatggatgagctcagtgaagatgataagttgaccgttgcccgtgcccgtaaaattcagcgattcttaagccagcctttccatgtggcagaagtcttcactggtgccccaggaaaatatgttgaattgaaggagaacatcactagtttccagggtgtgttggatggcaaatatgatgacctttcagagcaggcattttacatggttggtggtattgacgaggtcattgctaaggcagagaagattgcaaatgaatcacatgcctcagcttaatcacaattttattatcctttctctataattgctaaatttaataatttaggaactctttcttggcaatgccaaacaacaaaattttccgttattaaattttttataatttgaatggtcattcctttcatttgagtgttgttgccgacggtgagatgaaccaagttctttggtcaactgcccacttttttccgttgtgttatttaataagtggaagaggcaagaggatgtacatttttacttgtttaagaacgttggatgcatcactgttaaattttataactggttcatgtgttgtgaagcattctatttccttgctgaattttcaaccgtacaaaaatattattatgaa

SEQ ID NO: 70 - DNA, 2094 bp, Populus trichocarpa
aaagcagtgaatcacgtctactattatctgggacttacgagtttatgagctcacaccctctcacataccctaacatttcatctgtctttctctgaagccctaaccaaatggcttcacgcagacttttatcatctcttctccgatcgtcttctcgccgatctatctccaaatctactttatcaagtaccaatcctaaggtctctgcatcttccacgcgccgcgcgtctccttacggttacctcctaaaccgcgcggctgagtacgcgacctctgcagccgccgcatcgcctccatcacaaccgcctcctgtccaatcggagggtaagaaggggaaggtcatcgacgagtttaccgggaagggttcgatcgggcatgtgtgtcaggttattggagccgtcgtggatgtgaaatttgatgagggtttgcctccgattttgaccgctttggaggttcagggtcactcaatccggttggttcttgaagtggcccagcatttgggagagagtgttgttaggactattgctatggatggtactgaaggtttggtcagaggccagcctgttcttaacactggatctccaattactgtgcccgtgggcagggccactcttggtcgcataatcaatgtcattggagaagccattgatgagaagggagacctcaagacggagcactacttgcctattcatagggaggctccatcttttgttgagcaagcaactgagcaacaggtccttgtaactggtatcaaggttgttgatctccttgcaccataccaaagaggagggaagattggactgtttggcggtgctggcgtgggaaaaactgtgcttattatggaacttatcaacaatgttgcaaaagctcatggtggtttctctgtctttgctggtgttggtgaacgtacccgtgagggaaatgacttgtacagagaaatgattgaaagtggtgtcattaagctaggagatcagcaggctgagagcaaatgtgctcttgtgtatggtcaaatgaatgagcctcctggtgctcgtgcccgtgttggtctcactggacttactgtggctgagcacttccgtgatgctgaagggcaagatgtgcttctattcattgacaacattttccgctttacccaggctaactcagaggtgtctgccttgcttggacgtatcccatctgctgttggttatcagccaaccttggctacagatcttggaggtcttcaagaacgaattacaacaaccaagaaaggttccattacttctgttcaagctatttatgtgcctgctgatgatttgacggatcctgctcctgccacaacttttgctcacttggatgccacaactgtgctgtcaagacagatttctgagcttggtatctatcctgctgtggatccccttgattctacatctcgtatgctctcacctcatattttgggtgaggaacactacaacactgctcgtggtgtgcagaaggttcttcaaaactataagaatttgcaagatatcattgccattttgggaatggatgagctcagtgaagacgacaagctgacggttgcccgtgctcgtaaaattcagaggttcttgagccagcccttccatgttgcagaagttttcactggtgctcctggaaagtatgtcgagttgaaggagggtgtacaaagcttccagggagtgttggatgggaaatacgatgaccttccagagcagtcattttacatggttggtggtatcgaggaggttattgccaaggctgagaagatttccaaggaatctgctgcttaaacagaccatcttttttctccttttattatcataaactgatttttgcgaaaatcaataattaaggtggtgctggtgatgccagcagcagggcgttcattttttgagatcaaatgtgttacgatttgcaatttctgttttaatgctattgtagagtgtggaagaggaatagaactgagacctgtccagccaaacccccttttttcatgttgcttaaataagggggaaacagaggggggtgaacctgtatatttttcgcttttatcttcaggacatcaaatatcagttatcccttgaaaagcatatttctaat

SEQ ID NO: 71 - DNA, 2242 bp, Populus trichocarpa
atcagatccactattagctgagccttagctacatataacagtatatcggtctcacaaaccctaacattcacccctccctatctgtctccatcttcctctgaagccctaaccatggcttcacgcaggcttttatcttctcttctccgatcaacttctcgccgatctctctccaaatctaccctatcaaattctcaccctaagctctctacatcctccacgcgccgcgcgtcaccttacggctaccttctaaaccgcgcggctgagtacgcaacctctgcagccgccgcatcgccttcgtcacaaccgcctcctgccaaatcggagggaacgaaggggaagatcatcgatgagtttaccggcaagggttcgatcgggcatgtgtgtcaggttattggtgccgttgtggatgtgaagttcgacgagggtttgcctccgattttgacggcgttggaggttcagggacactcgatccggttggtccttgaagtggcgcagcatttgggtgagagtgttgttaggactattgctatggatggtactgaaggtttgattagaggccagcctgtgctaaacactgggtccccaattactgtgcctgtgggtagggccacccttggtcgtatcatcaatgtcattggagaagccattgacgagaagggggatctcaagacagagcactacttgcctattcatagggaggctcctgcttttgttgagcaagcaactgagcaacagatccttgttaccggtattaaggttgtcgatctccttgcaccataccaaagaggaggaaagattggactgtttggtggtgctggtgttggaaaaactgtgcttattatggaacttatcaacaatgttgcaaaagctcatggtggtttctctgtctttgctggtgttggagaacgtacccgtgagggaaatgacttgtacagagaaatgattgaaagtggtgtcattaagctaggggatcagcagtccgagagcaaatgtgctcttgtgtatggtcaaatgaacgagccccctggtgctcgtgctcgtgttggtctcactgggcttactgtggctgagcacttccgtgatgctgaagggcaagatgtgcttctcttcattgacaacattttccgctttacccaggctaactcagaggtgtctgcattgcttggtcgtatcccatctgctgtcggttatcagccaaccttggctacagatcttggaggtcttcaagagcggattacaacaactaagaaaggttctattacttctgtccaagctatttatgtgcctgctgatgatttgacagatccagctcctgccacaacctttgctcacttggatgccacaactgtgctgtcacgacagatttctgagcttggtatctatcctgctgtggatccccttgattctacatctcgtatgctctcacctcatattttgggcgaggaccactataacactgctcgtggtgtgcagaaggttcttcagaactataagaatttgcaagatattattgccattttgggaatggatgagctcagtgaagatgataagctgacagttgcccgtgctcgtaaaattcagaggttcttgagccagcccttccatgttgcagaagttttcaccggtgctcctggaaagtacgtagagttgaaggagggtgtagaaagcttccagggtgtgttggacgggaaatacgatgaccttccagagcagtcattttacatggttggcggtattgaggaggttattgccaaggctgaaaagatttccaaggaatctgctactgcttaaacagaccaacttttttctccttttcttcccttcaactgataattgcaaaaatcaataattaaggtggtgctggtgatgctagcaacaagatgttcattttttgagatcaaatttgtcaggatttgcaatttctgttttaatgttgttgcagagtgtgaaagaggaatagagctgagacctgtccagccaagcccccctttctccgtgttgcttgaatgagggggcaacagagggaggtgggcttgtatatttttcctttatcaactgtcaattatctcttgagaatctttctgacagcgtttacggataatcctgttttgaatattgcaaaactggttcaattgaacgcacgtctgttaacttgttctagtcgtggctggtacaaatttgtttggacagggcttttgaacggaatagtttctggttttggtaatcagaaattctgtttgca

SEQ ID NO: 72 - DNA, 2095 bp, Glycine max
caaaccctagccctcttctttctttctctctctctctctctctctctctgaagatggcttcacgcaggctcgtatcttctctgattcgatcctcccttcgtagatctcaatcgaagccctcaatttccgcatcgacatcgaggctcacgtcatccaaccgtgcctcaccgcacggatacttgctgaaccgcgtcgccgagtacgccaccgccgcggctgctgctaccactcctccctctcctcctcctccggggaagaaggagctcggcggcggcgggaagatcaccgatgaattcaccgggaagggcgcgatcgggcaggtctgccaggtcattggtgccgtcgtcgatgtcagattcgacgagggtttgcctccgatcatgaccgcgctggaggttctggatcactcgtcgaggcttgtgttggaggtggcgcagcatttgggtgaaggcgttgtccgaaccattgctatggatgccaccgaaggtgtcgttagaggctggcgcgttctcaacactggctcccctattaccgttccagttggtagggctacccttggccgtatcataaatgtcattggagagcctattgatgccaagggagaaatcaatactgagcattatttgcccattcatagagaagctcctgcttttgttgagcaagaaactgcacagcagattcttgttactggaatcaaggttgttgacctgcttgcaccatatcaaagaggaggaaagattgggttgtttggtggtgctggtgtaggaaaaactgtgcttattatggaacttattaacaatgttgcaaaagctcatggtggtttctctgtgtttgctggtgttggagaacgaacccgagagggtaatgacttgtacagagaaatgattgagagtggtgtcattaagcttgatgataagcagagtgaaagcaagtgtgctcttgtgtatggtcaaatgaatgagccccctggtgcccgtgcccgtgttggtcttactgggcttactgtggctgaacacttccgtgatgctgaagggcaagatgtgcttcttttcgtagacaacattttccgttttacccaagctaactcagaggtgtctgctttgcttggtcgtatcccatctgctgttggttaccaaccaaccttgtctactgatcttggagctcttcaagagcgtattacaacaaccaagaagggttcaattacctctgtccaagctatctatgtgcctgctgatgacttgacagatcctgctcctgctaccacttttgctcacttggatgccacaacagtgttgtcacgacagatctccgagcttggtatctatcctgctgttgatcccttggattctacatctcgtatgctttccccccttattttgggtgcggatcactacgaaactgctcgtggtgtacagaaagtgcttcagaactacaagaatcttcaagatatcattgctattttgggaatggatgagctcagtgaagatgataaattgactgttgcccgtgcccgtaagattcagcgattcttaagccagcctttccatgttgctgaagtcttcactggtgccccaggaaaatatgttgagttgaaggagaacgttgccagcttccagggtgtgttggatggaaaatacgatgaccttccagagcagtcgttttacatggttggcggtattgaggaggtcattgcaaaggccgagaaaattgctaaggaatctgcagcgtcttaatcacacacagccctgttctcattttaatcccttttttttttgtaattgctaaatttaataatttaggaactcttggcaatgccaaacaacaaaattttccattttttgggtatgaaaatgtatcaggttattccttccttccattttaatgttgccgactgtgagatgaactacggtcaatgtctgtggccaaactgcccccctgtttttaagttgtgttatttaataaggggaaaagggcaagaggatgtacattttttactttctggttgagaacgttggatgcattaatgttaaaaactctgcaactgttccacctggtccatggccagcgtaatttgagttccttactgaatattgga

SEQ ID NO: 73 - DNA, 2013 bp, Fragaria vesca
cctccttactctttcctctctgaagccctaaccagatggcttcccgtcggctcttatccactctcgcccgttcctccgttcgccgatctccgtccaaatctccgatctccaactcccaccccaggctcacctccccctcccctccgccccaccgcgcatcgccctacggctacctcctctcgcgcgtggccgagtacgccaccgccgcagccgccgagaccaagtcggtgtcgccggccgggccgaaggatggcgccaagggaaaaatcaccgacgagttcaccgggaaaggcgcgatcgggcaggtgtgccaggtcatcggagccgtcgtcgatgtgagattcgaggaggggttgccgccgatcttgacggcgctggaagtgctggacaactcgatccggttggtgcttgaggtggctcagcacttgggtgagagcatggtcaggactattgctatggatgggactgaggggcttgttagaggacagcgcgtgctcaacaccggctctcccatcactgtgcctgttggtaggtctacccttggtagaatcatgaatgttattggagagcccattgatcatagaggcgatatcacaaccgaacactatctgcccattcatagagaagctccagcctttgttgagcaagctactgaacaacagatccttgttactggaattaaggttgttgaccttcttgctccataccaaagaggaggaaagattggattgtttggtggtgctggtgtaggaaagacagtgcttattatggaactgatcaacaatgttgcaaaagctcatggtggtttctccgtgtttgccggtgttggagaacgcactcgtgagggtaatgacttgtacagagaaatgattgagagtggtgtcattaagctcggtgaaaagcaggctgacagcaaatgtgctctagtgtacggtcaaatgaacgagccccctggtgcccgtgctcgtgttgggttgactggcctgactgtggcagaacatttccgtgatgctgaagggcaagatgtgctcctctttatcgacaacattttccgatttacccaagctaactctgaggtgtctgctttgcttggtcgtatcccatccgctgtcggataccaacccactctagctactgatcttggaggtcttcaagagcgtatcacaaccaccaagaagggttccattacttctgtccaagctatttatgtgcctgctgatgacttgacagatcctgctcctgcaactacttttgctcacttggatgccacaactgtgttgtctagacagatctctgagcttggtatctatcctgctgtcgatccccttgattccacatctcgtatgctctctccccatattttgggtgaggaacattacaacactgctcgtggagtgcagaaggtccttcagaactacaagaatttgcaagatatcattgctattttgggaatggacgagcttagtgaagatgataaattgactgtcgcccgtgctcgtaagattcaacgtttcttgagccagccgttccatgtcgcagaagttttcaccggtgcccccggaaagtacgttgagttgaaagaaagcattcaaagcttccagggagtgttggatgggaagttcgatgacctttcagaacaatcattctacatggttggaggtattgaggaggttattgccaaggctgagaagattgccaaggaaaatgcttaagccacaccgctatctttgttcctttaccttgataattgcgaaaaccaaataatttagatggcgctggctatgccggcgaacaggattttcatttttgagacaaattgtatgagcatttcccatttcagattcaaggatattgctgagagtgtgagagaaatggagccaatggcccatcccctttcttcttgttttatttaataaggggaaaaaagatagaggacatataaatttattttaccttttcaattttccaaacccttggatgcagcagcactggatgttcaactctttgtttatg

SEQ ID NO: 74 - DNA, 2084 bp, Glycine max
cacaaaccctagtagccctcttcattctttctctcttctctctctgtccgaagatggcttcacgcaggttcgtatcttctctgattcgatcctcccttcgtagatctcaatcgaaaccctcgatttccgcatccgcatcgaggctcacgtcatccaaccgtgcctctccgcacggttacttgctgaaccgcgtcgccgaatacgctaccgcggcggcggctgctaccgctcctccctctgctccgcctccgggcaagaaggaggttagcggcggcgggaagatcaccgatgagttcaccgggaagggctcgatcgggcaggtctgccaggtcatcggtgccgtcgtcgatgtcagattcgacgagggtttgcctccgatcatgaccgcgctggaggttctggatcactcctcgaggctcgtgttggaggtggctcagcatttgggtgagggcgttgtccgaaccattgccatggatgccaccgaaggggtcgttagagggtggcgcgtcctcaacactggctcccctattaccgttccagttggtagggctacccttggccgtatcataaatgtcattggagagcctattgatgacaagggagaaatcaataccgagcattatttgcccattcatagagaagctcctgcttttgttgagcaagaaaccgcacagcagattcttgttactggaatcaaggttgttgacctgcttgcaccatatcaaagaggaggaaagattgggttgtttggtggtgctggtgtaggaaaaactgtgcttattatggaacttattaacaatgttgcaaaagctcatggtggtttctctgtgtttgctggtgttggagagcgaacccgagagggtaatgacttgtacagagaaatgattgagagtggtgtcattaagcttggtgataagcagagtgaaagcaaatgtgctcttgtgtatggtcaaatgaatgagccccctggtgctcgtgcccgtgttggtcttactgggcttactgtggctgaacacttccgtgatgctgaagggcaagatgtgcttctttttgtagacaacattttccgttttacccaagctaactcagaggtgtctgctttgcttggtcgtatcccatctgctgttggttaccaaccaaccttgtctactgatcttggagctcttcaagagcgtattacaacaaccaagaagggctcaattacctctgtccaagctatctatgtgcctgctgatgacttgacagatcctgctcctgctaccacttttgctcacttggatgccacaacagtgttatcacgacagatctccgagcttggtatctatcctgctgttgaccccttggattctacatctcgtatgctttccccccttattttgggtgcggatcactatgaaactgctcgtggtgtacagaaagtacttcagaactacaagaatcttcaagatatcattgctattttgggaatggatgagctcagtgaagatgataaattgactgttgcccgtgcccgtaagattcagcgattcttaagccagcctttccatgttgcagaagtcttcactggtgccccaggaaaatatgttgagttgaaggagaacatcaccagtttccagggtgtgttggatggcaaatacgatgacctcccagagcagtcgttttacatggttggcggtattgaagaggtcattgctaaggctgagaaaattgctaaggaatctgcagcgtcttaattacatacaacagccatgttctcattttaatcctttttttgtaattgctaaatttaataatttaggaactcttggcaatgccaaacgacaaaattttcaattttttgggtatgaaaatgtgtcaggttattcccttcccattttaatgttgcctactgtgagatgaactaaggtcaaagtctctggccaactgcccccctttttttaagttgtgttatttaataaggggaaaagggcaagaggatgtaaatttttactttttctggttgagaacgttggatgcattaatgttaaaactttgtaattgttccatctggtccatggccagcgtaatttttgttccttactgaa

SEQ ID NO: 75 - DNA, 3384 bp, Medicago truncatula
atggcttcacggagaatcgtttcgtctttgatacgttcctccctccgtccatctctatccaaatcatccatcaccacctcaacctcgagactctcttcttctcaatcacgtgcatctccctatgcttaccttcttaaccgtgtcaccgagtatgccacagccgccgctgctgcagctcccgctcctcctcagactcctccggctaagaaagttcctgatggtggcggtaagattacagatgaatttactggtaaaggtgcgattggacaggtttgtcaggtgattggtgctgtggtggatgtgagattcgaagaaggtttgcctccaattcttactgcgcttgaggttcttgatcattctacacgattggtgttggaggttgcacaacatatgggtgaaggtgttgtgagaactattgctatggatgccactgaaggagttgttagagggtggcgtgttctcaacaccggctcccctatcagtgttcccgttggcaggtgtactcttggacgtatcatgaatgttattggagaacctattgatcataaaggcgactttaaaaccgagcattttttgcctattcatagagaagctcctctttttgttgaacaagcaaccgaacaacaaattcttgtgaccggtatcaaggttgttgatctccttgcaccataccaaagaggaggaaagattggattgtttggtggtgctggtgtaggaaaaactgtgcttattatggaacttatcaacaatgtcgcaaaggctcatggtggtttctctgtttttgccggtgttggagaacgaacccgagagggtaacgacttgtatagggaaatgattgagagtggtgtcattaagcttggcgatcagcagagtgaaagcaaatgtgctcttgtttatggtcaaatgaacgagccccctggtgctcgtgcccgcgttggtcttacaggattgactgttgctgaacatttccgtgatgctgaaggacaagatgtgcttcttttcgtcgataacattttccgctttactcaagcaaactcagaggtgtctgcattgcttggtcgcatcccatctgccgttggttatcaaccaacgttgtctactgatcttggaggtcttcaagagcgtattacaaccaccaagaagggttcaattacctctgtccaagccatctatgtgcctgctgatgacttgacagatcctgctcctgctaccacctttgctcacttggatgctacgacggtgttgtcaagacagatctctgagcttggtatctatcctgctgttgatccattggattcaacatctcgtatgctttcaccacttattttgggtgatgagcattacgaaactgctcgaggtgtacaacaagtacttcaaaattacaagaatcttcaagatatcattgctattttgggaatggatgaactaagtgaagatgataaattgaccgttgcacgtgctcgtaaaattcaacgattcttaagtcagcccttccatgtggcagaagtcttcactggtgcccctggcaaatatgttgacttgaaagagaactgtactagtttcaagggtgtgttggatggcaaatacgatgacctttcagaacaagcattttacatggttggtggtattgatgaagtcattgcaaaggcagagaagattgctaaggaaaatgcgcatcttcattctctttctctctcatcgaagatggcttcacgcagattggtatcatctctgattcgatcttcccttcgccgatcttcatcgaaaccatcaatcactgcctcaacatcgaggctaacctcccaatctcgcgcttcaccttatggctacctcttaaaccgcgtcgctgattatgcaaccgcggctgccgccgcaccagctccgtctgcgcctccggcgaagaaggaggttcccggaggtgggaaaattactgatgaatttactggaaaaggagcggtcggtcaagtttgtcaggttattggtgccgtcgttgacgtcagatttgaagaaggtttgcctccgatcttgactgctcttgaagttctagatcattcttcgagattggtgttggaagttgctcagcatttgggtgaaggtgttgtgagaacgattgctatggatgctactgaaggtgttgttcgtggatggcgtgttcttaacaccggttctcccatcagtgttcccgttggcagggctacccttggacgtatcatgaatgttattggagaacctattgatcataaaggcgaattcattaccgagcattatttgcctattcatagagaagctcctgcttttgttgagcaagcaactgaacaacagatccttgttactggtatcaaggttgttgacctgcttgcaccataccaaaggggagggaagattgggttgtttggtggtgctggtgtaggaaaaaccgttcttattatggaacttatcaacaatgttgctaaggctcatggtggtttctctgtgtttgctggtgttggagaaagaacccgagagggtaatgacttgtacagagaaatgattgagagtggtgtcattaagctgggtgaaaagcagagtgagagcaaatgtgctcttgtctacggtcaaatgaatgagccccctggtgctcgtgcccgtgtcggtcttactggacttactgttgctgagcatttccgtgatgctgaaggacaagatgtgcttcttttcgttgacaacattttccgctttactcaagctaactcagaggtgtctgccctgcttggtcgtattccatctgcggttggttaccaaccaacattgtctactgatcttggaggtcttcaagagcgtattacaaccaccaagaagggttcaattacatctgtccaggctatctatgtgcctgctgatgacttgacagatcctgctcctgctaccacctttgctcacttggatgctacaacagtgttgtctagacagatttctgagcttggtatctatcctgctgttgatcctttggattcaacatctcgtatgctctctccacttattttgggtgatgagcactaccaaactgctcgtggtgtacaacaagttcttcaaaactacaagaatcttcaagatatcattgctattttgggaatggatgagcttagtgaagatgataaattgactgttgctcgtgcccgtaaaattcaacgattcttgagtcagcctttccacgtggcagaagttttcactggtgccccaggcaaatatgttgagttgaaggagaacactcaaagtttccagggtgtgttggatggcaaatatgatgacctttcagagcaggcattttatatggtcggtggtattgatgaagtcattgcaaaggcagagaagattgccaaggaatcagcagcatcttcatcttag

SEQ ID NO: 76 - DNA, 1043 bp, Eutrema salsugineum
tctgagagcaaatgtgctcttgtgtatggacaaatgaatgagcccccgggtgcccgtgctcgtgttggactgactggtttgactgttgctgagtatttctgtgacgctgaaggccaagatgtcttgcttttcattgataacattttccgcttcacccaggccaactccgaagtgtctgctttgctcggtcgtatcccgtctgctgtgggttaccagccaactttggcttccgatcttggtgctcttcaagagcgaatcacaaccaccaagaaaggttcaatcacctcagtccaagccatctatgtccctgctgatgatttgacagatcctgctctagccacaactttcgctcacttggacgccacaaccgtgctctcgagacagatttcagagcttggtatctatcctgctgtggatcctctggattcaacatcccgtatgctctcgcctcacattctaggtgaggagcactacaacacggctcgtggcgtgcagaaagtgctacagaactacaagaacttgcaagatattatcgccattttgggaatggatgagcttagtgaagatgacaagctgactgttgcccgtgcccgtaagatccagagattcttgagtcagcccttccatgtcgctgaaatcttcacgggtgcccctggaaaataagtggacctcaaagaaaacatcaacagtttccagggtttgcttgatggtaagtacgatgatcttcccgaacaatcattttacatggttggaggtatcgatgaggtggttgcaaaggcagagaagatttccaaggagtctgcagcttagaagctggattcatgtctttcacatacttgatacttcgtctgattattatcttttttcgacaataacgaaaagaaaaaccttatgcctgtgagtagtgccatgcctatagatgggtgtagaaagaaacatcggtctgcaggcccattttttaataaaattttgaaggtcattgccaactactatccagcaacagcaacgatatttcaagaaaaatgtggataaaaatgtgttgc

SEQ ID NO: 77 - DNA, 1893 bp, Cucumis sativus
gagcagtgtaggcgttgcatatagtgccattccactctcattcatcgaaccctatgctgagcttctgaagccctaacaagcaatggcttcacgcagactcttatcttcccttctccgatcgtcggcacggcggtcttcttccagatctcccatctcttcccccaaccccagaattccattatccaacacttctcgcgcttccccttatggctacctcctcaaccgtgtcgctcaatacgccacctccgccgccgccgccactccaccaccttcctccccaccatctgccaagtcggacgtaggaccgagtggaaagatcactgacgagttcactggagccggctcgatcggccaggtttgtcaggtgattggtgccgtcgtcgatgtcagattcgatgaagggttgcctccaattcttacggcgcttgaggtgcttgatcactcgatccgtttggtgctggaggtggcgcagcatttgggggagaatatggttaggactattgccatggatgggacggaagggcttgttcgtgggcggagagtgctcaatactggttctcctatcactgttccagttggtagagctactcttggacgtattattaatgtcatcggagagcctattgacgagaaaggcgaccttaagaccgaccactatttgcctattcacagagaagctccagcctttgttgagcaagcaactgagcagcagattctcgtaactggtattaaggttgttgaccttcttgctccatatcaaagaggaggaaagattgggctttttggtggtgctggtgtgggaaaaactgtgcttatcatggaacttatcaacaatgttgcgaaagctcatggtggtttctctgtgtttgctggtgtcggagaacgcactcgagagggtaatgatttatacagagaaatgattgagagtggtgtcattaagcttggtgataaacaggctgaaagcaaatgtgctcttgtgtacggtcaaatgaatgagcccccgggtgctcgtgctcgtgttggtcttactggactaactgtggccgaacacttccgtgatgctgaaggacaggatgtgcttctcttcattgacaacattttccgctttacccaggctaactcagaggtgtctgcactgcttggacgtattccatctgctgttggttaccaaccaaccttggctactgatctgggaggccttcaagaacgtattaccaccactaagaaaggatctattacttctgtgcaagctatctatgtccctgctgatgatttgactgatcctgcccctgctaccacctttgctcacttggatgccacaactgtgttgtcgcgtcagatttcagagcttggtatttatcccgctgtggatccccttgattctacatctcgtatgctctctcctcatattttgggagaggaccactataatactgctcgtggagtacagaaggttcttcagaattacaagaatttgcaagatattattgccattcttggaatggatgagctcagtgaagatgataagttaactgttgctcgggctcgtaaaatccaacgattcctgagccagcctttccatgttgctgaagtatttactggagcaccgggcaagtatgtggaactgaaagaaagtatcaccagtttccagggagttttggatggaaagtatgacgaccttcccgaacaatcattctacatgattggaggaatcgaagaggtcattgctaaggcagagaagattgccagggaatctgctgcttaacattgaggttgtcgtatttcacaactccccattttaatctttgtaattgtgaagatcaataatatttaggcgatgttggcaaatgttattctgtcaacaagattttcatttatataattttttttaat

SEQ ID NO: 78 - DNA, 2095 bp, Cucumis sativus
gagcagtgtaggcgttgcatatagtgccattccactctcattcatcgaaccctatgctgagcttctgaagccctaacaagcaatggcttcacgcagactcttatcttcccttctccgatcgtcggcacggcggtcttcttccagatctcccatctcttcccccaaccccagaattccattatccaacacttctcgcgcttccccttatggctacctcctcaaccgtgtcgctcaatacgccacctccgccgccgccgccactccaccaccttcctccccaccatctgccaagtcggacgtaggaccgagtggaaagatcactgacgagttcactggagccggctcgatcggccaggtttgtcaggtgattggtgccgtcgtcgatgtcagattcgatgaagggttgcctccaattcttacggcgcttgaggtgcttgatcactcgatccgtttggtgctggaggtggcgcagcatttgggggagaatatggttaggactattgccatggatgggacggaagggcttgttcgtgggcggagagtgctcaatactggttctcctatcactgttccagttggtagagctactcttggacgtattattaatgtcatcggagagcctattgacgagaaaggcgaccttaagaccgaccactatttgcctattcacagagaagctccagcctttgttgagcaagcaactgagcagcagattctcgtaactggtattaaggttgttgaccttcttgctccatatcaaagaggaggaaagattgggctttttggtggtgctggtgtgggaaaaactgtgcttatcatggaacttatcaacaatgttgcgaaagctcatggtggtttctctgtgtttgctggtgtcggagaacgcactcgagagggtaatgatttatacagagaaatgattgagagtggtgtcattaagcttggtgataaacaggctgaaagcaaatgtgctcttgtgtacggtcaaatgaatgagcccccgggtgctcgtgctcgtgttggtcttactggactaactgtggccgaacacttccgtgatgctgaaggacaggatgtgcttctcttcattgacaacattttccgctttacccaggctaactcagaggtgtctgcactgcttggacgtattccatctgctgttggttaccaaccaaccttggctactgatctgggaggccttcaagaacgtattaccaccactaagaaaggatctattacttctgtgcaagctatctatgtccctgctgatgatttgactgatcctgcccctgctaccacctttgctcacttggatgccacaactgtgttgtcgcgtcagatttcagagcttggtatttatcccgctgtggatccccttgattctacatctcgtatgctctctcctcatattttgggagaggaccactataatactgctcgtggagtacagaaggttcttcagaattacaagaatttgcaagatattattgccattcttggaatggatgagctcagtgaagatgataagttaactgttgctcgggctcgtaaaatccaacgattcctgagccagcctttccatgttgctgaagtatttactggagcaccgggcaagtatgtggaactgaaagaaagtatcaccagtttccagggagttttggatggaaagtatgacgaccttcccgaacaatcattctacatgattggaggaatcgaagaggtcattgctaaggcagagaagattgccagggaatctgctgcttaacattgaggttgtcgtatttcacaactccccattttaatctttgtaattgtgaagatcaataatatttaggcgatgttggcaaatgttattctgtcaacaagattttcatttatataatttttttttaatctttttggagatgtcttgtaccaagatcctccccttgccattttagtgtgttgaagagaaatgaaacctctggcctgccccttgttttttgttgtttgaataaggggggaaaatggactcagaactttgtacactttttcccaatcgattccaaaacattggatgccagtttagaagtttattatggttattattaccatt

SEQ ID NO: 79 - DNA, 1138 bp, Eutrema salsugineum
tggtgtcatcaagctaggcgagaagcagtcagagagcaaatgcgctcttgtgtatggacaaatgaatgagcccccgggtgctcgtgcccgtgttggacttactggattgactgtcgctgagtatttccgtgatgctgaaggccaagatgtcttgctttttatcgacaacattttccgtttcactcaggccaactcagaagtgtctgctttacttggacgtatcccgtctgctgtcggttaccagccaactttggcttccgatcttggtgctcttcaggagcgaatcacaaccactaagaaaggttctatcacctcagtccaagccatctatgtccctgctgatgatttgactgatcctgctccagccacgacttttgctcacttggacgccacaaccgtgctctcgagacagatatctgagcttggtatctatcctgctgtggatcctctggattcgacatcccgtatgctctcacctcacattctgggtgaggatcactacaacacggctcgtggggtgcagaaagtcctacagaactacaagaacttgcaagatattatcgccattttgggaatggatgagcttagtgaagatgacaagttgactgttgcccgtgcccgtaagatccagagattcttgagtcagccattccatgttgctgagatcttcacgggtgccccaggaaaatacgttgatcttaaggaaaacatcaacagtttccagggtttgctcgatggtaagtacgatgatcttcctgaacaatcgttttacatggttggtgggattgacgaggtggttgcaaaggcagagaagatctccaaggagtcagcagcttaagcgcctgattcatctctcttggatgtttaatacttctctttttcttcttttgttgacaataacgaaaagaaaaaaaacaaaaccaatgctttaggctgagctgctaagtgcattgcgattcccatcattgtgagtgtgagagccgggagatgatgctgaacatattcatttacggttactcaaatttttggttcttttgcttcctttacttttgaaataaaggtcaagtctttcagattcgaaattgcaatgtgtgatttaagatttcgtgatatcaatctcttatttctctttgattca

SEQ ID NO: 80 - DNA, 2123 bp, Vitis vinifera
attaaaatcatcttgaggcgcacaggcgcagtggtaatagttaagcagagtaggtcgtatagagagaggcactaaccctattgtctctcctcggaaaccctagagccatggcttctcggaggctcttatcttctcttcttcgatcgtccgtacgccgatattcggccaaatctccggcaatcagccctagaactccatcgccttacccttcttctcgcccttctccaaagggttttctcctcaaccgcgctgttgactatgcaacgtcggccgctgccgcggcatcaccaccgccgccaccgcccgcgaagggagcggggccgagcgggaaaatcactgacgagttcactggcgccggtgcgattggcagtgtctgtcaggtgatcggtgccgtcgtcgatgtcagattcgatgaggggttgccaccgatcttgacggccctggaggttctggacaattcgattcggttggtgttggaggttgcccagcatttgggagagaacatggtgaggaccattgctatggatggaacggaagggctcgtgcgtggacagagagtcctcaatactggttctcctatcactgtgcctgttggtagggctacccttggccgcatcattaatgttattggagagcctatcgatgaaaggggcgaaatcaaaaccgaccactttttgcctattcatcgagaagctccatcctttgttgatcaagccactgaacaacagatcctcgttactggaatcaaggttgtcgatctccttgcgccataccaaagaggtggaaagattgggctgtttggtggtgctggagtaggaaaaactgtgcttattatggaacttataaacaatgttgccaaagctcatggtggtttttctgtgtttgctggtgttggagagcgtactcgagagggtaatgacttgtacagggaaatgattgagagtggtgtgattaagcttggggataagcagagcgaaagcaagtgtgcacttgtgtatggtcaaatgaatgagccccctggtgcccgtgctcgtgttgggcttactggattgactgtggctgagcactttcgtgatgctgaaggacaagatgtgcttctctttattgacaacattttccgctttacccaagctaactctgaggtgtctgctttgcttggtcgtattccatctgctgtcggttatcaaccaaccttagcaacagatcttggaggccttcaagagcgtattacaacaaccaagaaaggctccatcacatctgtccaagccatttatgtgcctgctgatgacttgactgatccagctcctgcaaccacctttgctcacttggatgccacaactgtgttgtcccgacagatctctgagcttggtatctatcctgctgttgatcctcttgattctacatctcgtatgctttccccacatattttaggagaggagcattacaacacagctcgtggggtacaaaaggttcttcagaactacaaaaatcttcaagatatcattgccattttgggaatggatgagctcagtgaagatgataagttgactgttgcccgtgctcgtaaaattcaacgattcttgagccagcctttccacgttgcagaagttttcactggtgcccctggaaagtatgtggagttgaaagagagcattaccagcttccagggagttttggatggaaaatatgatgacctttcagaacagtccttctacatggttggaggaattgaagaagtcattgctaaagcagagaagattgcaaaggaatctgctgcctaattaagttgcatttccccactgactattttgttcctttgtctcgataattgcgaaaatcaataatttagttggtgttggtaattccatcaccgggagtttcatttttttgaggtcaaatgtgtgaggatttccatttctgttatatattgttgcagggttgtgatagagaaatggatcccaaaggcttgccctgtcaagcccctttttattttgttattcaataagcaaaaacgaagagggctggcatgtatatttttcttgctttttatctattggaatcttggatgcccaacctacaaatacacatgcctttgactcacatcaaacatatcgtgcctttca

SEQ ID NO: 81 - DNA, 2057 bp, Fragaria vesca
acatttcgatagccccagtagctctctctctctctctctctcctcacagtttttggtgagtgattcaaactccaaatgagctcttgatccataaacccctcatttccgatcaaatttaccaaacctgccaccatgttctcctcccgcaagctcctctccaccctcctccgctcctctctccgccgcacctcctccgccgccgctaggcccccactctccagatcctcccccatcacgcgctcccccggctacctcctctcatgcgccgctcactactccacctcatccgccgccgccgtctccgcgcctcccaaatccggcacgccgtcgacgaccggcaagatcaccgatgagtttaccggcaaaggcgccatcggccaggtctgccaggtcatcggcgccgtcgtcgatgtccggttccaggacggcttgccgccgatcctgacggcgctcgaggttttggataactcgatccggttggtgctcgaggtggctcagcatctcggtgagaatatggttaggactatcgccatggatgggactgaagggctcgttagaggtcaacgagtcctcaacactggctctcccatcactgtgcctgttggaagagcaaccctgggccgaattatgaatgttattggcgagcccattgatcataggggtgaaataaataccgatcactctctgcccatccatagagaagcaccggcctttgttgagcaggccactgagcaacagattcttgtcactggaatcaaggttgttgatcttcttgctccttaccaaagaggaggaaagattgggctctttggtggtgctggtgttgggaagaccgtgcttattatggaacttatcaacaatgttgccaaagctcatggtggtttctctgtgtttgctggtgtgggagaacgtacaagagagggtaatgatttataccgagaaatgattgaaagtggtgtcattaagctcggagaaaagcagagtgatagcaagtgtgctcttgtatatggtcaaatgaatgaacctcctggtgcccgtgctcgtgttgggctcactgggctgactgtggctgagcatttccgtgacgctgaagggcaagatgtgctactcttcatcgataacatattccgctttacccaagctaactcagaagtgtctgctttgctcggtcgtatcccatctgctgtcggttaccaacctacgttggctactgatcttggaggacttcaagagcgtatcactacaaccaagaagggttccattacgtctgttcaagctatttatgtacctgctgatgacttgacagatccagctcctgcaaccacttttgcccatcttgatgccaccactgtgctgtcacgacaaatatctgagcttggaatctatcctgctgtcgaccccttggattctacatctcgcatgctctcacctcatattttaggagaagaacattacaacactgctcgtggtgttcagaaagttcttcaaaactataagaatctgcaggatattattgcaattttgggtatggatgagcttagtgaagatgacaaattaactgttgctcgtgctcgtaagatccaacgtttcttgagtcaacctttccatgtggctgaagtgttcacgggagctcctggaaaatatgtggagttgaaagagagcattaccagtttccagggagttctggatgggaagtatgatgatctttctgagcaatcgttctatatggttggtggaattgaagaagtcattgccaaggcagagaagatagccaaggaaaatgcttaggtcatgtggctattatctgtcatcctgagtttccaaagagctaataatgtcggcattggtaattctcaggaatttcatttgttttgaggagagaactggctttttcagttaaagcattaattttgttagggttgtattaggtaaagagatgaggagtgggcattttctgttaggccccttcccttgtgcaataaggtatgcacttgtgtgtatttacttgcaaattataatcagttcagttcacaaggaaatgcgcctcttgtcttgtt

SEQ ID NO: 82 - DNA, 1221 bp, Eutrema salsugineum
tctgagagcaaatgtgctcttgtgtatggacaaatgaatgagcccccgggtgcccgtgctcgtgttggactgactggtttgactgttgctgagtatttctgtgacgctgaaggccaagatgtcttgcttttcattgataacattttccgcttcacccaggccaactccgaagtgtctgctttgctcggtcgtatcccgtctgctgtgggttaccagccaactttggcttccgatcttggtgctcttcaagagcgaatcacaaccaccaagaaaggttcaatcacctcagtccaagccatctatgtccctgctgatgatttgacagatcctgctctagccacaactttcgctcacttggacgccacaaccgtgctctcgagacagatttcagagcttggtatctatcctgctgtggatcctctggattcaacatcccgtatgctctcgcctcacattctaggtgaggagcactacaacacggctcgtggcgtgcagaaagtgctacagaactacaagaacttgcaagatattatcgccattttgggaatggatgagcttagtgaagatgacaagctgactgttgcccgtgcccgtaagatccagagattcttgagtcagcccttccatgtcgctgaaatcttcacgggtgcccctggaaaataagtggacctcaaagaaaacatcaacagtttccaggtacatgtttactggccgtggattgagctaaaatttgtttttagtatgcaagaatcctttgtgcttgctaggtctatgatgggctagttttattctgccgtagagaaattttgttatcaaatgtaataagtcaccaggctgacccagatcatcttgtggggttcttgatttgtttaagggtttgcttgatggtaagtacgatgatcttcccgaacaatcattttacatggttggaggtatcgatgaggtggttgcaaaggcagagaagatttccaaggagtctgcagcttagaagctggattcatgtctttcacatacttgatacttcgtctgattattatcttttttcgacaataacgaaaagaaaaaccttatgcctgtgagtagtgccatgcctatagatgggtgtagaaagaaacatcggtctgcaggcccattttttaataaaattttgaaggtcattgccaactactatccagcaacagcaacgatatttcaagaaaaatgtggataaaaatgtgttgc

SEQ ID NO: 83 - DNA, 1973 bp, Oryza sativa
aaaagtctaaccctagatccaggccccgcgtctccggcgatctcccggccatggcgactcgccgggccctctcctccctcgtccgcgccgcctccaggctccgcggggcctcgcccgccccgcgcccgcgcgggccgctccaccgaccgtcgccatcggggtacctcttcaaccgcgccgccgcgtacgccacggccgccgcggcgaaggaggcggcgcctcccgcgcccgcgacggggaaggccacgggtggaggtaagatcaccgacgagttcaccggcgccggcgccgtcgggcaggtgtgccaggtcatcggcgccgtcgtcgacgtgcggtttgacgaggggctgcctcccatcctcacggcgctcgaggtgctcgaccacaacatccgcctcgtgctcgaggtggcgcagcaccttggcgagaacatggtgcgcaccatcgctatggacgggactgaggggcttgtccgcggtcagcgcgtcctcaacaccggctccccaatcactgttcctgttggcagggccacgcttggacgtatcatgaatgttattggtgagccaattgatgagaagggtgacataacaacgaaccacttccttcccatccatcgtgaggcgcctgcttttgttgagcaagccacagaacagcaaattcttgttactggaattaaggttgtggatctgcttgcgccctaccaaagaggtggaaagatcggtctttttggtggtgcaggagtcggcaaaactgtccttattatggagttgatcaacaatgttgctaaggcccatggtggtttctctgtgtttgctggtgttggtgaacgtacccgtgaaggtaatgatctttacagggaaatgattgaaagtggtgtcatcaagctaggtgacaaacagagtgaaagcaagtgtgctcttgtctacgggcaaatgaatgagcccccgggtgctcgtgctcgtgttgggttgaccggtttgactgttgcggaacatttccgtgatgccgaaggacaagatgtgcttttgttcattgacaacattttccgtttcactcaggcgaactctgaggtgtctgctcttcttggacgtattccatctgctgtgggatatcaaccaactcttgctactgatcttggaggacttcaagagcgaattacaactacaaagaagggttccattacatctgtccaagctatttatgtgcctgctgatgacttgacggatcctgctcctgctactacttttgcacatcttgatgctactactgtgttgtcacgacagatctctgagcttggtatttaccctgctgtcgatcctctggactccacatccagaatgctctccccccatgttttgggtgaggatcactacaacactgctcgtggtgtccaaaaggttcttcagaactacaagaatcttcaggatattattgcaattttgggtatggacgagctcagtgaagatgacaagttgaccgtcgctcgcgcaaggaagatccagcgtttcttgagccagcccttccatgtggctgaagttttcacgggtgctcctgggaagtacgtggagctgaaggagagcgtcaacagtttccagggtgttttggatgggaaatatgatgaccttcccgagcagtcattctatatggtgggaggcattgaggaagtcattgctaaagctgagaagatcgccaaggagtcggcttcataagatctcttcattgttaagttctgtacaagttaaatttttggattctggtttctatatgcccgttctgtagccaggacgtgattacaagcaggggtgacatcttctgatgagcgatgtttttttgtcttccttttctccttttacccctaataaggaaccgcagtgacattgtattgtgctgcacaaaagtactcggaacttcgaaagcatgtgtcctttataatgcagatccatgaagcataggatcattgagttcttgttttt

SEQ ID NO: 84 - DNA, 1643 bp, Oryza brachyantha
gcgcttcgacgagggcctgccgcctatcctgacggcgctcgaggtgctcgacaacaacatccgcctcgtgctcgaggtggcgcagcaccttggcgagaacatggtgcgcaccatcgctatggacgggactgaggggcttgtccgcgggcagcgcgtcctcaacacaggctccccgatcactgttcctgttggcagggccacacttggacgcatcatgaatgttattggtgagccgattgatgagaagggtgacataacaacgaaccacttccttcccatccatcgtgaggctcctgcttttgtcgagcaagccacagaacagcaaattcttgttactggtattaaggttgtggatctgcttgcgccctaccaaagaggtggaaagattggtcttttcggtggtgcaggagtgggcaaaactgtccttattatggagttgatcaacaatgttgctaaggcccatggtggtttctctgtgtttgctggtgtcggtgaacgcacacgtgaaggtaatgatctttacagagagatgattgagagtggtgtcatcaagctaggtgacaaacagagtgaaagcaagtgtgctcttgtctatgggcaaatgaatgagccaccgggtgcccgtgctcgtgttgggttgactggtttgactgttgctgaacatttccgtgatgctgaaggacaagatgtgcttctgttcattgacaacattttccgtttcactcaggcaaactctgaggtgtctgctcttcttgggcgtattccatctgctgtgggataccaacctactcttgccactgatcttggtggattacaagagcgtattacaactaccaagaagggttctattacatcagtccaagctatttacgtgcctgctgatgacttgacggatcctgctcctgctaccacttttgcacatcttgatgctactaccgtgttgtcacgacagatctctgaacttggtatttaccctgctgtcgatcctctggactccacatccagaatgctctcaccccacgtgttgggtgaggatcactacaacactgctcgtggtgtccaaaaggttctccagaactacaagaatcttcaggatattattgcaattttgggtatggatgaactcagtgaagatgataagttgactgtcgctcgtgcaaggaagatccagcgtttcttgagccagcctttccatgtggccgaagttttcacgggtgctcctgggaagtatgtggagctgaaggagagtgtcaacagtttccagggtgttctggatgggaaatatgatgaccttcccgagcagtcattctacatggttggaggcattgaggaagtcattgctaaagctgagaagattgccaaggagtcggcttcataagatctcttcattgtttgagttctgtacaagttaaatttttggattccggtttctatatgcccgttactgtagccaggacgtgcttacaagcagaggggtgacatcttctcatgaggaatgttttttgtcttctttttttgcccttttacccctaataagaaatcgcagtgacattgtttgttctgcacaaaagtacccgacttggaaaacatgtgccctttataatgcagatccatcaaatcatcggatcatgaagttcttgttt

SEQ ID NO: 85 - DNA, 1692 bp, Oryza brachyantha
cccgcgcctccccccgcgtcgggcaaggccaccggtggtggcaagatcaccgatgagttcacgggcgccggcgccgtggggcaggtgtgccaggtgattggtgccgtcgtggacgtgcggttcgacgagggcctgcccccgatcctgacggcgctggaggtgctcgacaacaacatccgcctcgtgctcgaggtggcgcagcatctcggggagaacatggtgcgcaccatcgccatggacgggaccgagggcctcgttcgtggccagcacgtgctcaacaccggctctcccatcactgttcctgttggtagggctacacttgggcgtattatgaatgtcattggtgagcctattgatgagaagggagatataacgacaaaccactttctccctatccatcgtgaagctcctgcttttgttgagcaagctacagagcaacagatcctcgttactggaattaaggtcgtggatctccttgcaccgtatcaaagaggtggtaaaattggtctcttcggtggtgcaggggtgggcaaaactgtgcttattatggagttgatcaacaatgtcgcgaaggctcatggtggtttttctgtctttgctggtgttggagaacgtactcgtgagggtaatgacttgtacagggaaatgattgagagtggtgtgattaagcttggtgacaagcagagtgagagcaaatgtgctcttgtctacgggcaaatgaatgagccccccggtgctcgtgctcgtgttggactgactggtttgactgttgccgagcatttccgtgatgctgaagggcaggatgtgcttctcttcatcgataacattttccgtttcactcaggcaaactctgaggtgtctgccctgcttggacgtatcccatctgccgtgggataccagccaacccttgctactgatcttggaggactgcaggagcggattaccaccacaaagaagggttctatcacatctgttcaagctatttatgtgcctgctgatgacttgacagatcctgcccctgctactacatttgcccatcttgatgctactactgtgttgtcacgacagatttctgagcttggtatttatcctgctgtcgatcctttggattccacatccagaatgttgtcaccccatgtgctgggtgaggaccactacaacacagctcgtggtgttcagaaggttcttcagaactacaagaatcttcaagatattattgccattctgggaatggatgagctcagtgaggatgataagttgacagtcgctcgtgctaggaagattcagcggttccttagccagcccttccatgttgctgaagttttcacgggtgcacctggaaagtatgtcgagctaaaggagagtgttcaaagtttccagggtgtgttggatgggaaatatgatgacctgccggagcagtcattctacatggttggaggaattgatgaggtcattgccaaggcagagaagattgccaaggagtccgcttcataagcgatgcaatttttaacatttggaggtcaacatgttgtcttttatgtttgttagccaggacatgtacttacatgcaggaggcccggccctgctgatcgatgttttcccctccactttttttccaaataaggatttgcagtgacctatttgtctggcttactgcccttcaaggaagaattgagttctctttttgtttga

SEQ ID NO: 86 - DNA, 2059 bp, Brachypodium distachyon
atcctttcccgaagtgcggagccgccactccccaccccgcggaccatttggaaaacctagatccaaatcctcgccgccggccatggcgacccgccgggccatctcatccatcgtgcgttccgcttcccgcatccgtgccgcctccccgtccccgttaccccgcgcgccgctccaccgcccgtcccccgccgggttccttctcaaccgcgccgccgcctacgcgacctccgccgcggcgcaggctgctcccgccacgccgccccccacctccgctaagcacactggtggtaagattacagatgagttcaccggcgccggctcggtggggcaggtttgccaggtgattggcgctgtggtcgacgtgcggttcgacgaggggctcccgcctatcctaacggcgctggaggtgctcgacaacagtatccgccttgtgctcgaggtggcgcaacatctcggggagaacgtggtccgcaccatcgctatggacgggacggaagggctcgtccgcggtcagcgcgtgctcaacaccggctctcccatcactgtccctgttggtagggcaactcttgggcgtattataaatgttattggtgagccaatcgatgagaggggagatataaagacaaaccatttcctacctatccatcgtgaagcccctgcttttgttgagcaagctactgagcaacaaatccttgttactggaattaaggttgtggatttgcttgcaccctatcaaagaggtggtaaaattggtctctttggtggtgcaggggtgggcaaaactgtccttattatggagttgatcaacaacgttgccaaggcccatggtggtttctctgtctttgctggtgttggagaacgtactcgtgagggtaacgacttgtatagggaaatgattgagagtggtgtgattaagcttggtgacaagcagagtgagagcaagtgtgctcttgtctacgggcaaatgaatgagccccctggtgctcgtgcccgtgttggactgactggtttaactgttgctgagcacttccgtgatgctgaaggacaggatgtgcttctcttcattgataacattttccgtttcactcaggcaaactctgaggtgtctgccctgcttggacgtattccatctgctgtgggataccaaccaacccttgctactgatctgggaggactacaggaacggattaccaccaccaaaaagggttccattacatctgtgcaagctatttatgtgcctgctgatgacttgacagatcctgcacctgctacaactttcgctcatcttgatgctactactgtgctctcacgacagatttctgagcttggtatttatcctgctgtcgatcctttggactccacatccagaatgttgtctccccacgttcttggtgaggagcactacaacactgctcgtggtgtccagaaggttctccagaattacaagaatcttcaagatattattgccattttgggaatggatgagctcagtgaggatgataagttaacagttgctcgcgctaggaaaattcagcggttccttagccagccttttcatgttgctgaagtgttcacaggtgcacctggaaagtatgttgaactaaaggagagtgttcaaagtttccagggtgtattggatggcaagtacgatgatctttctgagcagtcattttacatggttggtggaatagatgaggtcattgccaaggcagagaagatcgccaaggagaatgcgtagaaatctcctcacattttaactttttgtggtcaacctgttgtgccttttattttatgttagccaggatgtacttacaggcaggaggcccggccctgctgatggatgttttcacccctccactttttcccgaataaggacttgcagtgacctatttgtttggcttactgcccttcaaggaagaattgagttctcttttttgtgtgacccatgagcggcgaggccgttaacctgttacttctcattaaaaaaaaactgaaatatttgtcttcgatagtagtgctctcctgtctatacgtggtacgtgttgatgtttctattcaa

SEQ ID NO: 87 - DNA, 4390 bp, Setaria italica
tccccaccccgaccccgattcccaaaccctccaccctttcccgtagcgcggagccgccactccccacacccgcacaccactctgaggaaaacctagatccggacccctcgccgccggccatggcgacgcgccgggccctctcctccatcctccgctccgcctctcgcctccgcgcagcctccccgtccccggtcccgcgcgcgccgctccaccaccgcccgtcccccgcgggcttcctcctcaaccgcgccgcctcctacgcctcctccgccgcggcccaggcggcgcccgccccgccgcccccgtccactggcaagacaaccggtggcggcaagatcaccgatgagttcaccggcgccggcgccatcggccaggtgtgccaggtcatcggtgccgtcgtggacgttcggttcgacgagggtctgcccccgatcctgacggcgctcgaggtgctcgacaacaacatccgcctcgtgctcgaggtggctcagcatctcggggagaacatggtccgcaccatcgccatggacgggactgaggggctcgtccgcggccagcgtgtgctcaacaccggctctcccatcactgtccctgttggtagggcaacacttggacgcattatgaatgttattggcgagcctattgatgagaagggagatataacgacgaaccacttcctccctatccatcgtgaagccccagctttcgttgagcaagctacagagcagcaaattcttgttactggaatcaaggttgtggatctccttgcaccctaccaaagaggaggtaaaatcggtctctttggtggtgcaggggtgggcaaaactgtccttatcatggagctcattaacaacgttgccaaggcccatggtggtttctctgtcttcgctggtgttggagaacgtactcgtgagggtaatgacttgtacagggaaatgattgagagtggtgtcattaagcttggtgacaagcagagtgagagcaagtgcgctcttgtctacgggcaaatgaatgagccccctggtgctcgtgctcgtgttggactgactggtttgactgttgccgagcatttccgtgatgctgaaggacaggatgtgcttctcttcattgataacattttccgtttcacccaggcgaactctgaggtgtctgctctgctgggtcgtattccatctgctgtgggataccaacccacccttgccacggatcttggaggactgcaggagcggattaccaccacaaagaagggttctattacatctgttcaagctatttatgttcctgctgatgacttgacagatcctgcccctgcaaccaccttcgcccatcttgatgccactaccgtgttgtcacgtcagatttctgagcttggtatttaccctgctgtcgaccctctggactccacatccagaatgttgtccccccacgtgctgggtgaggaccactacaacactgctcgtggtgttcagaaggttcttcagaattacaagaacctccaagatattattgccattttgggaatggatgagctcagtgaagatgacaagttgacggtcgctcgtgctaggaagattcagcggttccttagccagcctttccatgttgctgaagttttcacgggtgcacctggaaagtatgttgagctgaaggagagtgttaaaagtttccagggtgttttggatggcaagtatgatgacctcccagagcagtcattctacatggttggaggaatagaggaggttattgctaaggcagagaagattgccaaggagtctttaaagatatcccccaacggctctctgctgctgccgactgtctccttcccgaaccaacggatttccaaaccccaaatctcctccgcccttcttgcctcctcctactccccctaccctatttcaaaacatttcaaacactctccgcctccttcctcccatccaatccccttcccattccgttctctcctctcgcccctgaggcgccggatcgcgcgattccctccacggcgacagccggccgccccctccccgtcccgcccgctcgacgaggccacgccgagcggtgaagagggagccactggggtcgctcgccctcacgcgatgagtagcgccgtgaaggaacagctccaacagatgtcgacgacgtgcgattcgctcctgctggagctcaacgtgatttgggatgaggtcggggagccccactcggcgagggaccggatgctgctggagctcgagcaggagtgtctcgaggtctacaggaggaaggtcgacatggcaaaccgctgcagggcacagctgcggcaggccatcgccgaggcagaggccgagctcgcaggtatctgctcggccatgggtgagccgccgatacatgttagacagtcaaatcagaagttgcaaggtttaagggaggaattgaacgcgattgtcccatacttggaggagatgagaaagaagaaagttgaaagatgggaccaatttgttgatgtcatagatcaaattaagaaggttgcatctgaaatcaggcctgcagattttgtgccctttaaatttcctgtggatcaatccgatctgtcagtaagaaagcttgaggagttaacaaaggagctacaatcccttcagaaggagaagagtgatcggctgaagcaagtgatggaacatttgaacactttgcattccttgtgcgaggtgcttggtatagacttcaaacaaacagtacatgatgtgcatcctagcctgggcgaggctgatggatcaaagaacctgagcaacagtacaattgagagactcgcatcagctgtaaatggattacgtgaaatgaaagtccagaggatgcaaaagcttcaagatttggcatctagcatgcttgaactttggaatctcatggatacaccacttgaagagcagcagatgtttcagaatgtaacatgcaatattgctgcttcggaacatgaaataactgagcccaacaccctctctgttgactacctcagctacgtcgaatctgaagttttaaggcttgaacaattgaaagcgagcaagatgaaagacctggttctgaaaaagaagacagaactcgaagaacataggagacgtgctcatctgatcggtgaggaaggttatgcagctgaatttagcactgaggctattgaagcaggagctgttgatcctgcgctggttctggaacaaattgaggctcacattgctacagtgaaagaggaagcttttagccgaaaggatattcttgagaaggttgaaagatggctgaatgcatgtgaggaggaagcctggttggaagattataacaaagatgacaatcgttataatgctgggaggggggcccatctgacactcaagagagcagaaaaggctcgtattttggttaacaagatcccaggaatggtagatgttttgaccacgaaaattttagcttggcagaaagaaagagaaaaggaattcacatatgatggtgtccgccttctgtcaatgcttgaagagtacatgatcgttcgtcaggagaaagagctagagaagaagaggcaaagggatcagaagaaaatccaggatcaaatcaaagctgagcaggaagcactctacggatcaaaaccaagtccatccaagcctcaaagtacaaagaaggtgcctaggaactccatgggtggtgcaaaccgaaggctgtctcttggcggagccacaatgcaagccccaaagacagacatactgcattcaaagactgctcgtgctgccaagaagactgaagaattgggcactttatctcctagtagtagaggcttggacattgctggtcttcccatcaagaagttaactttcaatgcaagtactctgcgtgagacagaaacaccgcgcaagccttttgcccagatcatgccaggaaacaatgtctcatcgacacctgcacggccaaccttcaatgacaacaccgaggaagagaacaaaacccccaagacatttaatgcagcgcttgatctcaaaacgccgatgacggtgacggctccgatgcagctggcgatgacgccttctgtgggtaacaaggtcatagccactcctgtctcccttttccaggagaaaccagagcagccgatgctgccggaggagatcgagtactcgttcgaagagaggcggctcgccgtttaccttgccaggcaaatggcttagctcagccatttgccccgtcattacatagttgaaagtcgaactgactgccatttctcgtcagaggccaatggtacctgtttgctggtcgacaatagtcggcctttttcgcactttgccgtactgttctgattcacagccatatttggtacaattagttaaggagttccacacttgtgagcttgtgctccgtgccgccattggttatcttaagttgtatagct

SEQ ID NO: 88 - DNA, 2049 bp, Oryza sativa
attccccgcagcgcggagccgccgccactcccccccaccctcaccccgcagaccattcggaaaacctagatccgagccttcccccttcccctcccccggcggcggcggccatggcgacccgccgggccctcacctccgtcctccgctccgcctcccgcctccgcgccgcctccccttccccgtgcccgcgccgcgcgccgctccacccccaccgccgcccgtcccccgcgggcttcctcctgaaccgcgccgccgccgcctacgcgtcgtccgccgcggcgcaggcggcccccgcgcctccccccgcgtcgggcaaggccaccgggggcggcaagatcaccgatgagttcacgggcgctggcgccgtcgggcaggtgtgccaggtgattggcgccgtcgtggacgtgcggttcgacgagggcctgcccccgatcctgaccgcgctggaggtgctcgaccacaacatccgcctcgtgctcgaggtggcgcagcatctcggggagaacatggtgcgcaccatcgccatggacgggaccgaggggctcgttcgcggccagcgcgtgctcaacaccggctctcccatcactgttcctgttggtagggccacacttgggcgtattatgaatgtcatcggtgagcctattgatgagaagggagatatcacaacaaaccacttcctccctatccatcgtgaagcccctgcttttgttgagcaagctacagagcaacagattcttgttactggaattaaggtcgtggatctccttgcaccctatcaaagaggtggtaaaattggtctcttcggtggtgctggggtgggcaaaactgtacttattatggagttgatcaacaatgttgcgaaggctcatggtggtttctctgtctttgctggtgttggagaacgtactcgtgagggtaatgacttgtacagggaaatgattgagagtggtgtgattaagcttggtgacaagcagagtgagagcaagtgtgctcttgtctacgggcaaatgaatgagcccccgggtgctcgtgctcgtgttggactgactggtttgactgttgccgagcatttccgtgatgctgaagggcaggatgtgcttctcttcattgataacattttccgtttcactcaggcaaactccgaggtgtctgccctgcttggacgtatcccatctgctgtgggataccagccaacccttgctactgatcttggaggactgcaggagcggattaccaccacaaagaagggttctatcacatctgttcaagctatttatgtgcctgctgatgacttgacagatcctgcccctgctactacatttgcccatcttgatgctactactgtgttgtcacgacagatttctgagcttggtatttatcctgctgtcgatcctttggattccacatccagaatgttgtctccccatgtactgggtgaggaccactacaacacagctcgtggtgttcagaaggttcttcagaattacaagaatcttcaagatattattgccattctgggaatggacgagctcagtgaggatgataagttgacagtcgctcgtgctaggaagattcagcggttccttagccagcccttccacgttgccgaagttttcacgggtgcacctggaaagtatgttgagctgaaggagagcgttcaaagtttccagggtgtattggacgggaagtatgatgaccttcctgagcagtcattctacatggttggaggaatcgatgaggtcattgccaaggcagagaagattgccaaggagtccgcttcataagcgatgcaatttttaacatttggaggtcaacatgttgtgccttttatgtttgttagccaggacatgtacttacatgcaggaggcccggccctgctgatcgatgtttttcccctccacttttttccaaataaggatttgcagtgacctatttgtttggcttactgcccttcaaggaagaattgagttctctttttgtgtgatgtgcggccagtccggcagtccgtgaacttgttacttgctgaaaacactgaaatattggtctccc

SEQ ID NO: 89 - DNA, 1900 bp, Solanum tuberosum
ttcacctctatcctcactggtgtcactcgcatttccacagccatggcttctcgaaggttcttctcctccatgctccgctcatccatacgtcactcttcaactaaatcctcactcacaagttctatttatcgctcctcccctgcaggccaccttctccaccgcgccgttaaatatgctaccgctgcggccgcgaaggaagcgccggcgcctcagaagaaaccgacgacgattaaggaaaccggtggcaagatcaccgatgagtacaccggcgccggtgcacttgggagtgtatgtcaggtgatcggcgctgttgtggatgttcggttcgatgaaggactgccgccgattttgacggctttagaggtgttggatcatgatattagggtggtacttgaagttgctcaacatatgggggaaaatatggttaggactattgctatggatggaactgaaggacttgtgcgtggtcaaagagtcctcaatactggctcccctattagagtgcctgttggcagggctacacttggtcgtattataaatgtcattggagagcccattgatgaaaggggtgatctaaaaacggaacattatctcccaattcaccgtgaagctccatcttttgttgagcaagcaacagaacaacaaatccttgtgactggaatcaaggtggtagatctacttgctccgtatcaaagaggtggaaagattggactttttggtggtgcaggtgttggaaagacggtgcttattatggagcttattaacaatgttgcaaaggcccatggtggtttctcagtgtttgctggtgttggtgaacgtactcgggagggtaatgatttgtacagagaaatgattgagagtggtgttattaagcttggtgaaaagcagggtgagagcaaatgtgctttggtatatggtcaaatgaatgaacctcctggtgctcgtgctcgcgttgggctcactggactgacagttgccgaacacttccgagatgctgaagggcaagatgtgctccttttcatcgataatatttttcgcttcactcaagccaactctgaggtgtctgctttgcttggtcgtatcccctctgcagtcggttaccagccaactttagctacagatcttggaggacttcaagaaaggattactacaaccaagaaggggtctatcacatcagtccaagctatctatgtgcctgctgatgatttgactgatccagcacctgctaccacctttgctcaccttgatgctacaactgtgttgtctcggcagatttctgagcttggtatttatcctgctgtggatcctttagattctacgtcccgtatgctttctcctcatatcctaggtgaagatcattacaacactgcacgaggtgtacaaaaggttctccagaactacaagaatctccaggatattattgccattctgggaatggatgaattgagtgaagatgacaaactaactgttgctcgtgcccgtaaaattcagaggttcttgagtcagcctttccacgttgcagaagtatttactggtgcccctggaaagtatgtagagttgaaagagagcatccaaagttttcagggtgtcctggacggtaaatatgatgacctatcggagcaatcgttttatctggttgggggtatagaagaagtcattgctaaggctgagaagatagcaaaggagtcagctagttgatttagctgcaatttcttgtttctgtttgttttgtttttcctatatggataaatagcacattaaatagcagagagactggagatgctcaaacggccttttagttcttataagttagtctacactaagtattgattttcttctcttctccttgtagttttctagttaagttgattgaaatgaattacaggatgtcagaaggtca

SEQ ID NO: 90 - DNA, 1974 bp, Solanum lycopersicum
aacccatacaaatgtgaattcacctgtaacctcactgctctgtcactcgcatttccacaaccatggcttctcgaaggttcttctcctccatgctccgatcatccatacgtcactcttcaactaaatcatcactcacaaattctattcatcgctcctcccctgtaggccaccttctccaccgcgccgtaaaatacgctaccgctgcggccgcgaaggaagcgccggcgcctcagaagaaaccgacgacgattaagggaactggaggcaagatcaccgatgagtacaccggtgccggtgcacttgggagcgtatgtcaggtgatcggggctgttgtggatgttcggttcgatgaagggctaccgccgattttgacggctttagaggtgttggatcatgatattagggtggtacttgaagttgctcaacatatgggagaaaatatggttaggactattgctatggatggaactgaagggcttgtgcgtggtcaaagagtcctcaatactggctcccctattaaagtgccagttggcagggctacacttggtcgtattataaatgtcattggagagcccattgatgaaaggggtgatctaaaaacggaacattatctcccaattcatcgtgaagctccatcttttgttgagcaagcaacagaacagcagatccttgtgactggaatcaaggtggtagatctacttgctccgtatcaaagaggtggaaagattggactttttggtggtgcaggtgttggaaagacggtgcttattatggagcttattaacaatgttgcaaaggcccatggtggtttctcagtgtttgctggtgtcggtgaacgtactcgggagggtaatgatttgtacagagaaatgattgagagtggtgttattaagcttggtgaaaagcagggtgaaagcaaatgtgctttggtatatggtcaaatgaatgaacctcctggtgctcgtgctcgtgttgggctcactgggctgacagttgcagaacacttccgagatgctgaagggcaagatgtgctccttttcattgataatatttttcgcttcactcaagccaactctgaggtgtctgctttgcttggtcgtattccctctgcagtcggttaccagccaactttagctacggatcttggagggcttcaagagaggattactacaaccaagaaggggtcaatcacatcagtccaagctatctatgtgcctgctgatgacttgactgatccagcccctgctaccacctttgctcatcttgatgctacaactgttttgtctcggcagatttctgagcttggtatttatcctgctgtggatcctttagattccacgtcccgtatgctttctcctcatatcctaggggaagatcattacaacactgcacgaggtgtacaaaaggttctccagaactacaagaatcttcaggatattatcgccattctgggaatggatgaattgagtgaagatgacaaattaactgttgctcgtgcccgtaaaattcagaggttcttgagtcagcctttccacgttgcagaagtatttactggtgcccctggaaagtatgtagagttgaaagagagcatccaaagttttcagggtgtccttgacggtaaatatgatgacctatcggagcaatcgttctacctggttgggggtatagaagaagtcattgctaaggctgagaagatagcaaaggagtcagctagttgattatttagctgcaatttcttctttctgtttgttttgtttttcctacatggatagatagctcattaaatagcagagagactggagatgctcacacggccttttatagttcttataagttagtctacactaagaattgattttcttcttttctccttgtaattttctggctgagttgattgaaatgagttacaggatgtcagaaggtcattgcttcttttcgtgtttctcttcaatacaatggattaatttgtgttta

SEQ ID NO: 91 - DNA, 851 bp, Eutrema salsugineum
ccctagcatttcctctttcacacacacacccacaaactcatggcgtctcggagagtcttatcatcgcttctccgttcatcttccggcagatctgctgccaaattcgggagccggagccccaggattccttctccttcccctgctcgttgcgcttctcctttcggagacctcctcggccgagtcgccgagtattcgacctcttcaccggcaagctcagctgcgccatcgtctgcccctgctaaggatgaggggaagaagacctatgattacggtggaaaaggtgcgatcgggaaggtttgccaggtcattggtgccattgttgatgtaagattcgaggatcaggagggattgcctccgatcatgacatctctcgaggtgcaagaccatcccacgaggctggtgctcgaggtgtctcatcacttgggtcagaatgtcgtcaggaccattgcgatggatggtaccgagggtctcgtccgtggaaggcgcgttctcaacactggcgctccaatcactgtccctgttggaagagctacccttggacgtatcatgaatgttcttggtgaacccatcgatgagagaggcgaaattaagaccgaccattacttacctattcacagagatgctccggctttagttgatctggccactgggcaagaaatcctggcaactggtattaaggttgttgatctgcttgctccttaccaaagaggaggaaagattgggctttttggcggtgctggtgttgggaaaactgtgctcattatggaactgattaacaatgtcgctaaagcgcatggtggtttctctgtgtttgctggtgtgggagaacgaacccgtgaaggcaatgacct

SEQ ID NO: 92 - DNA, 2129 bp, Sorghum bicolor
aaacccctaccacccttccccgcagagtggagccgccactgccacccgcaccattctgaggaaaacctagatccggacccctcgccgccggccatggccacgcgccgggccctctcatctatcctccgctccgcctctcgcctccgctccgcctcaccgtccccgtgcccgcgcgcgccgctccaccaccgcccgtcccccgcgggcttcatactcaaccgcgcagccgcctacgcatcctccgccgcggcccaggccgcgcccgctccgccgcctccttccaccggcaagaaaaccggtggcggcaagatcaccgatgagttcaccggcgccggtgccatcggccaggtgtgccaggtcatcggcgctgtcgtggacgtgcgcttcgacgaggggctgcccccgatcctgacggcgctcgaggtgctcgacaacaacattcgcctcgtgctcgaggtggcgcaacatctcggtgagaacatggtccgtaccatcgccatggacgggacagaggggctcgtccgcggccagcgtgttctcaacaccggctctcccatcactgtccctgttggtagggcaacgcttggacgcattatcaatgttatcggtgagcctattgatgagaggggagatataacaacaaatcacttccttcctatccatcgtgaagccccagcctttgtcgagcaagctacagagcagcaaattcttgttactggaatcaaggtcgtggatcttcttgcaccttaccaaagaggtggtaaaattggtctctttggtggtgcaggggtggggaaaactgtccttatcatggagctgattaacaatgttgccaaggcccatggtggtttctctgtctttgctggtgttggagaacgtactcgcgagggtaatgacttgtacagagaaatgattgagagtggtgtcattaagcttggtgataagcagagtgagagcaagtgcgctcttgtctacggtcaaatgaatgagcctcctggtgctcgtgctcgagttgggctgactggtttgactgttgctgagcatttccgtgatgctgaaggacaggatgtgcttctcttcatcgataacattttccgtttcacccaggcgaactctgaggtgtctgctctgcttgggcgtattccatctgctgtgggataccaacccacccttgctactgatcttggaggactgcaggagcggattaccaccacaaagaagggttctattacatctgttcaagctatttatgtgcctgctgatgacttgacagatcctgctcctgcaaccacctttgcccatcttgatgctactactgtgttgtcacgtcagatttctgagcttggtatttaccctgccgtcgatcctctcgactccacatccagaatgttgtccccccatgttctgggtgaggatcactacaacactgctcgtggtgttcagaaggttcttcagaattacaagaatctccaagatattattgccattctgggaatggatgagctcagtgaagatgacaagttgacggtcgctcgtgctaggaagattcagcggttccttagccagccattccatgttgctgaagttttcacgggtgcacctggttatgtgttgtttagcggtgttttggatggcaagtatgatgaccttccagaacagtcattctacatggttggaggaatagaggaggtcatcgccaaggcagagaagattgccaaggaaaatgcatcataaacagtctccacaggcaatgcaaatttaactttggaggtcaacatgttgtgccttttattctgcgttatcaagaacatgtatttacgtgagcaggaggccctgccctgccaatggatgtgttttccctccgtttccccccataataaggattcactatgacattttcatttggcttactgccctttgaggaagaattatcctttttgatgacggcgaaactatgaacttgttaattctcaaagaaatactgtagtctcacatgttcatagctgttttgaggttacctgttcaatttttttctgaaccgattccacagtttgttaactgtgcagtctattttttgtgatctgtttccaactccagtgtttggttcatgggacgataagggatgagat

SEQ ID NO: 93 - DNA, 1461 bp, Physcomitrella patens
atgtccacagataacaaatcgacggacttcaccggcgctggaagcgtgggttccattgcgacagtgatcggagccgtggtggacgtgaagtttgagaagggcttgccacccattctgaccgccttggaggtgcaggaccacaacctgagggttgtattggaagtggcgcagcatttgggagagaacactgtgcggacgattgctatggaaacgaccgatgggcttgttcgcgggcagcgagtgttgaacaccgggtctcctatcatggtccccgtggggcgtgcaactttgggtcgtattatcaatgtcattggtgaagctatcgacgagagaggcgatatcagccgagagcatgttcttggaattcatcgtgaggcccctgccttcgtggaccaaggaacagagatggagattttggagactggaatcaaggtcgttgacctattggcgccctaccagcgaggtggtaagattggactgttcggcggtgcaggagtggggaagacggtgctgatcatggagctgatcaataacattgcaaaggcccacggaggattttcagtgttcgctggagtaggagagcgcacccgtgagggtaacgatctgtacaaggagatgattgagagtggtgtgatcaagctgggtgacaagcagatggagagcaagtgcgctctggtgtatggacagatgaacgagccccctggtgcccgtgcccgagtgggtctgactggactgactgtggcagagcacttccgtgatgctgaggggcaggatgtgttgttctttatcgacaacattttccgtttcacccaagcgaattcggaggtgtctgctctgttgggtcgtatcccatctgctgtggggtaccagcctactctggcgactgatctgggaggactccaagagcgaatcacgacaacgcagaagggatcgatcacctcggttcaggctatttatgtgcccgccgatgatttgacggatcctgccccagcgactacttttgcccatctggacgccacgactgtgctgtctcgtcaaatttctgagctgggtatttaccctgctgtggatcctttggattcgacctcccgtatgctgtccccgcgcattttgggagaggagcattactactgcgcacgagatgtgcagaaggtgctgcaaaactacaagaacttgcaggatatcattgccatcttgggtatggacgagctgagcgaagacgacaagttgactgtggcacgcgcgaggaagatgcagcgatttttgagtcaacccttccacgtggccgaggtattcacagggtcgcctggaaagtacgtggacttgaaagacagcgttgctggattccggggtgtgttggatggaaagtacgacgacttgcctgaacaggccttctacatggttggcggcattgaggaggtttcacagaaggcagagaagctggctaaggatatggcataa

SEQ ID NO: 94 - DNA, 342 bp, Eutrema salsugineum
atggcgagtcggcgaatcttatcatcgcttctccgttcatcttcagctgcgccatcgtcttctcctgctaaggatgaggggaagaagacctatgattacggtggaaaaggtgcgatcgggaaggtttgccaggtcattggtgtcattattgatgtgagattcgaggatcaggagggattgcctccgatcatgacatctctcgaggtgcaagaccatcccacgaggttggtgctcgaggtgtctcatcacttgggtcagaatgtcgtcagaaccattgctatggatggtaccgagggtctcgttcgtgggaggcgcgttctcaacactggcgctccaatcact

SEQ ID NO: 95 - DNA, 342 bp, Eutrema salsugineum; identical to SEQ ID NO: 94

SEQ ID NO: 96 - DNA, 326 bp, Arabidopsis lyrata
ataaaaggcaaacagcgagcatgtacaagaatgctaagaagaagattgagagagatgttgctttccctacatgcatttctgtgattcatttctcaccgcttgccagtgatgagtctgtgttggaagatggtgatatggttaaaataaggtggctgcagcttatgactgcaaaattgttgaaggtggctgcagcttaatcgtccaacccattatccagaaccccaagggtttgttggatggcaagtacgatgatctttccgaacaatcgttttacatggttggaggtatcgatgaggtggttgcaaaggcagagaagatcgctaaag

SEQ ID NO: 97 - protein, 287 aa, Arabidopsis lyrata
MGNLFCCVLVKQSDVAVKERFGKFQKILNPGLQFVPWVIGDYVAGTLTLRLQQLDVQCETKTKDNVFVTVVASIQYRVLVDKASDAFYRLSNPTTQIKAYVFDVIRACVPKLNLDDVFEQKNEIAKSVEEELDKAMTAYGYEILQTLIIDIEPDQQVKRAMNEINAAARMRVAANEKAEAEKIIQIKRAEGEAESKYLSGLGIARQRQAIVDGLRDSVLGFAGNVPGTSAKDVLDMVMMTQYFDTMRDIGATSKASAVFIPHGPGAVSDVATQIRNGLLQANNASSS

SEQ ID NO: 98 - protein, 284 aa, Capsella rubella
MGNLFCCVLVKQSDVAIKERFGKFQKVLNPGLQFVPWVIGDYVAGTLTLRLQQLDVQCETKTKDNVFVTVVASIQYRVLVDKASDAFYRLSNPNTQIKAYVFDVIRACVPKLNLDDVFEQKNEIAKSVEEELDKAMTAYGYEILQTLIIDIEPDQQVKRAMNEINAAARMRVAANEKAEAEKIIQIKRAEGEAESKYLSGLGIARQRQAIVDGLRDSVLGFAGNVPGTSAKDVLDMVMMTQYFDTMRDIGATSKSSAVFIPHGPGAVSDVATQIRNGLLQANNA

SEQ ID NO: 99 - protein, 285 aa, Eutrema salsugineum
MGNLFCCVLVKQSDVAIKERFGKFEKVLNPGLQFVPWIVGDYVAGHLTLRLQQLDVQCETKSKDNVFVTVVASIQYRVLVDKASDAFYRLSNPTTQIKAYVFDVIRACVPKLNLDDVFEQKNEIAKSVEEELDKAMTAYGYEILQTLIIDIEPDQQVKRAMNEINAAARMRVAANEKAEAEKIIQIKRAEGEAESKYLSGLGIARQRQAIVDGLRDSVLGFAGNVPGTSAKDVLDMVMMTQYFDTMRDIGAHSKSSSVFIPHGPGAVADVATQIRNGLLQAHQTS

SEQ ID NO: 100 - protein, 285 aa, Citrus sinensis
MGNLFCCVQVDQSTVAIKERFGKFEDVLEPGCHFLPWILGHQLAGHLTLRLQQLDVRCETKTKDNVFVNVVASVQYRALAHKANDAFYKLSNTRTQIQAYVFDVIRASIPKLNLDDAFEQKNEIAKAVEEELEKAMSAYGYEIVQTLIVDIEPDEHVKRAMNEINAAARLRVAANEKAEAEKILQIKRAEGEAESKYLSGLGIARQRQAIVDGLRDSVLGFSINVPGTTAKDVMDMVLVTQYFDTMKEIGAASKSSAVFIPHGPGAVRDVATQIRDGLLQASQHQ

SEQ ID NO: 101 - protein, 285 aa, Citrus clementina
MGNLFCCVQVDQSTVAIKERFGKFEDVLEPGCHFLPWILGHQLAGHLTLRLQQLDVRCETKTKDNVFVNVVASVQYRALAHKADDAFYKLSNTRTQIQAYVFDVIRAGIPKLNLDDAFEQKNEIAKAVEEELEKAMSAYGYEIVQTLIVDIEPDEHVKRAMNEINAAARLRVAANEKAEAEKILQIKRAEGEAESKYLSGLGIARQRQAIVDGLRDSVLGFSINVPGTTAKDVMDMVLVTQYFDTMKEIGAASKSSAVFIPHGPGAVRDVATQIRDGLLQASQHQ

SEQ ID NO: 102 - protein, 290 aa, Solanum tuberosum
MGNLFCCVQVDQSTVAIKERFGKFDDVLEPGCHFLPWCIGSQVAGYLTLRLQQLDVRCETKSKDNVFVTVVASIQYRALADKATDAFYKLSNTRSQIQAYVFDVIRASVPKLNLDDVFEQKNQIAKAVEDELEKAMSAYGYEIVQTLIVDIEPDEHVKRAMNEINAAARLRVAANEKAEAEKIVQIKRAEGDAEAKYLAGLGIARQRQAIVDGLRDSVLGFSVNVPGTTAKDVLDMVLITQYFDTMKEIGASSKSSAVFIPHGPGAVSDIAGQIRQGLLQGSAVEQQNLL

SEQ ID NO: 103 - protein, 285 aa, Solanum tuberosum
MGNLLCCVQVDQSTVAIKEQFGKYQDVLQPGCHFLPWCFGFQLAGHLSLRLQQLDVRCETKTKDNVFVNVVASIQYRALADKANDAFYKLSNTKGQIQAYVFDVIRASVPKLNLDDVFEQKNEIAKAVEDELEKAMSAYGYEIVQTLIVDIEPDEHVKRAMNEINAAARMRVAANEKAEAEKILQIKRAEGEAESKYLSGLGIARQRQAIVDGLRDSVLGFSVNVPGTSAKDVMDMVLLTQYFDTMKEIGASSKSSAVFLPHGPGAVRDVASQIRDGLLQASVDH

SEQ ID NO: 104 - protein, 294 aa, Setaria italica
MGKLPAAIGRFFCFVQVNQSTVGIKERFGKFEEVLNPGCHFMPWIIGNRVTGQLTLRLRQLDVRCETKTKDNVFVTVVASIQYRAMEDKASDAYYKLSNPKAQIQSYVFDVIRASVPKLELDDAFEQKNEIAKAVEEELEKAMSAYGYEIVQTLIVDIEPDEKVKRAMNEINAAARLRVAANEKAEAEKIIQIKRAEGEAEAKYLSGLGIARQRQAIVDGLRDSVLGFSGNVPGTSAKDVMDLVLLTQYFDTMKEIGAASKSSAVFLPHGPGAVADIASQIRDGFLQASTQQAK

SEQ ID NO: 105 - protein, 290 aa, Solanum lycopersicum
MGNLFCCVQVDQSTVAIKERFGKFDDVLEPGCHFLPWCIGSQVAGYLTLRLQQLDVRCETKSKDNVFLTVVASIQYRALADKATDAFYKLSNTRSQIQAYVFDVIRASVPKLNLDDVFEQKNQIAKAVEDELEKAMSAYGYEIVQTLIVDIEPDEHVKRAMNEINAAARLRVAANEKAEADKIVQIKRAEGDAEAKYLAGLGVARQRQAIVDGLRDSVLGFSVNVPGTTAKDVVDMVLITQYFDTMKEIGASSKSSAVFIPHGPGAVSDIAGQIRQGLLQGSAVEQQNLL

SEQ ID NO: 106 - protein, 287 aa, Capsella rubella
MGNLFCCVQVDQSTVAIKETFGKFEDVLEPGCHFLPWCLGSQVAGYLSLRLQQLDVRCETKTKDNVFVNVVASIQYRALANKANDAFYKLSNTRSQIQAYVFDVIRASVPKLLLDDVFEQKNEIAKAVEEELEKAMSAYGFEIVQTLIVDIEPDEHVKRAMNEINAAARMRLAANEKAEAEKILQIKRAEGEAEAKYLSGLGIARQRQAIVDGLRDSVLGFAVNVPGTTAKDVMDMVLVTQYFDTMKEIGAHSKSSAVFIPHGPGAVRDVATQIRDGLLQGSSANLS

SEQ ID NO: 107 - protein, 284 aa, Amborella trichopoda
MGQLFCCLQVDQSNVAIREQFGKFDDVLEPGCHFLPWCLGSQVAGYLTLRVQQLDVRCETKTKDNVFVTVVASIQYRALADKASDAFYKLSNTKNQIQAYVFDVIRASVPKLNLDDAFEQKNEIAKAVEEELEKAMSAYGYEIVQTLIVDIEPDARVKQAMNEINAASRMRVAANEKAEAEKILQIKRAEGEAESKYLSGLGIARQRQAIVDGLRESVIAFSSNVPGTSAKDVMDMVLVTQYFDTMKEIGASAKSSSVFIPHGPGAVRDVASQIRDGLLQANLG

SEQ ID NO: 108 - protein, 294 aa, Oryza brachyantha
MGKLVAAIGKLLCCVQVNQSTVGIKERFGKYEEVLDPGCHCVPWIIGSRVAGELTLRLRQLDVRCETKTKDNVFVTVVASIQYRAMEDKASDAYYKLSNPKTQIQSYVFDVIRASIPKLELDDAFLQKNEIAKAVEEELEKAMLAYGYEIVQTLIVDIEPDEKVKRAMNEINAAARLRVAANEKAEAEKIIQIKRAEGEAEAKYLSGLGIARQRQAIVDGLRDSVLGFSGNVPGTSAKDVMDLVLLTQYFDTMKEIGSTSKSSAIFLPHGPGAVADIASQIRDGCLQASSHHTK

SEQ ID NO: 109 - protein, 286 aa, Arabidopsis thaliana
MGNLFCCVQVDQSTVAIKETFGKFEDVLEPGCHFLPWCLGSQVAGYLSLRVQQLDVRCETKTKDNVFVNVVASIQYRALANKANDAYYKLSNTRGQIQAYVFDVIRASVPKLLLDDVFEQKNDIAKAVEEELEKAMSAYGYEIVQTLIVDIEPDEHVKRAMNEINAAARMRLAANEKAEAEKILQIKRAEGEAESKYLSGLGIARQRQAIVDGLRDSVLGFAVNVPGTTAKDVMDMVLVTQYFDTMKEIGASSKSSAVFIPHGPGAVRDVASQIRDGLLQGSSANL

SEQ ID NO: 110 - protein, 322 aa, Capsella rubella
SSRESAPPKRVFLFISFLCFKISTKKEENFWFVKKMGNLFCCVQVDQSTVAIKETFGKFEDVLEPGCHFLPWCLGSQVAGYLSLRLQQLDVRCETKTKDNVFVNVVASIQYRALANKANDAFYKLSNTRSQIQAYVFDVIRASVPKLLLDDVFEQKNEIAKAVEEELEKAMSAYGFEIVQTLIVDIEPDEHVKRAMNEINAAARMRLAANEKAEAEKILQIKRAEGEAEAKYLSGLGIARQRQAIVDGLRDSVLGFAVNVPGTTAKDVMDMVLVTQYFDTMKEIGAHSKSSAVFIPHGPGAVRDVATQIRDGLLQGSSANLS

SEQ ID NO: 111 - protein, 284 aa, Citrus clementina
MGNAFCCVQVDQSTVAIRERFGKFDEVLDPGCHCVPWLLGCQIAGDLSLRLQQLDVRCETKTKDNVFVNVVASVQYRALAKKASDAFYKLSNTRTQIQAYVFDVIRASVPKLDLDDVFEQKNDIAKAVEEELEKAMSAYGYEIVQTLIVDIEPDVHVKRAMNEINAAARLRVAANEKAEAEKIVQIKRAEGEAESKYLAGVGIARQRQAIVDGLRDSVLGFSENVPGTTAKDILDMVLITQYFDTMKEIGASSKSSSVFIPHGPGAVQDIATQIRNGLLQASSH

SEQ ID NO: 112 - protein, 291 aa, Vitis vinifera
MSAENMGNLLGCIQVDQSTVAIKERFGKFEEVLEPGCHCLPWCFGSQLAGHLSLRLQQLDVRCETKTKDNVFVNVVASIQYRALADKANDAFYKLSNTRSQIQAYVFDVIRASVPKLNLDDAFEQKNEIAKSVEDELEKAMSAYGYEIVQTLIVDIEPDEHVKRAMNEINAAARMRVAANEKAEAEKILQIKRAEGEAESKYLSGLGIARQRQAIVDGLRDSVLGFSVNVPGTTAKDVMDMVLVTQYFDTMKEIGAASKSSAVFIPHGPGAVRDVATQIRDGLLQSTLSHQ

SEQ ID NO: 113 - protein, 286 aa, Vitis vinifera
MGNLLGCIQVDQSTVAIKERFGKFEEVLEPGCHCLPWCFGSQLAGHLSLRLQQLDVRCETKTKDNVFVNVVASIQYRALADKANDAFYKLSNTRSQIQAYVFDVIRASVPKLNLDDAFEQKNEIAKSVEDELEKAMSAYGYEIVQTLIVDIEPDEHVKRAMNEINAAARMRVAANEKAEAEKILQIKRAEGEAESKYLSGLGIARQRQAIVDGLRDSVLGFSVNVPGTTAKDVMDMVLVTQYFDTMKEIGAASKSSAVFIPHGPGAVRDVATQIRDGLLQSTLSHQ

SEQ ID NO: 114 - protein, 287 aa, Arabidopsis lyrata
MGNLLCCVQVDQSTVAIKETFGKFEEVLEPGCHFLPWCLGSQVAGYLSLRVQQLDVRCETKTKDNVFVNVVASIQYRALANKANDAYYKLSNTRSQIQAYVFDGIRASVPKLLLDDVFEQKNDIAKAVEEELEKAMSAYGYEIVQTLIVDIEPDEHVKRAMNEINAAARMRLAANEKAEAEKILQIKRAEGEAESKYLSGLGIARQRQAIVDGLRDSVLGFAVNVPGTTAKDVMDMVLVTQYFDTMKEIGASSKSSAVFIPHGPGAVRDVATQIRDGLLQGSSANLS

SEQ ID NO: 115 - protein, 292 aa, Oryza sativa
MGKLVAAIGKLLCCVQVDQSTVGIKERFGKYEEVLDPGCHCVPWIIGSRVAGELTLRLRQLDVRCETKTKDNVFVTVVASIQYRAMEDKASDAYYKLSNPKSQIQSYVFDVIRASIPKLELDDAFLQKNEIARAVEEELEKAMLAYGYEIVQTLIVDIEPDEKVKRAMNEINAAARLRVAANEKAEAEKIIQIKRAEGEAEAKYLSGLGIARQRQAIVDGLRDSVLGFSGNVPGTSAKDVMDLVLLTQYFDTMKEIGSTSKSSAIFLPHGPGAVADIASQIRDGCLQAHQTK

SEQ ID NO: 116 - protein, 285 aa, Populus trichocarpa
MGNLLGCVKVDQSTVVIKERFGKFNEVLEPGCHCMPWFLGSQVAGHLTLRLQQLDVRCETKTKDNVFVNVVASVQYRALAHKASDAFYKLTNTRSQIQAYVFDVIRASVPKLLLDDVFEQKNEIARAVEEELEKAMSAYGYEIVQTLIVDIEPDEHVKRAMNEINAAARMRLAANEKAEAEKIIQIKRAEGEAESKYLAGLGIARQRQAIVDGLRDSVLGFSDNVPGTSAKDVLDMVLITQYFDTMKEIGASSKSSAVFIPHGPGSVRDIATQIRDGLLQASAPQ

SEQ ID NO: 117 - protein, 284 aa, Glycine max
MGNLFCCVQVDQSTVAIREGFGRFEKVLQPGCHCMPWFLGKQLAGHLSLRLQQLDLRCETKTKDNVFVNVVASIQYRALAEKANDAFYKLSNTKTQIQAYVFDVIRASVPKLNLDDAFEQKNEIAKAVEEELEKAMSAYGYEIVQTLIVDIDPDEHVKRAMNEINAAARLRMAANEKAEAEKILLIKRAEGEAESKYLSGLGIARQRQAIVDGLRDSVLGFSVNVPGTTAKDVMDMVLVTQYFDTMKEIGAASKSSAVFIPHGPGAVRDVAGQIREGLLQASHQ

SEQ ID NO: 118 - protein, 284 aa, Fragaria vesca
MGNLLCCVQVDQSTVAMREQFGKFVDVMEPGCHCMPWFLGHQVAGHLSLRLQQLDVRCETKTKDNVFVNVVASIQYRALADKANDAFYKLSNTRSQIQAYVFDVIRASVPKLLLDDAFEQKNEIAKAVEDELEKAMSAYGYEIVQTLIVDIEPDEHVKRAMNEINAAARMRVAANEKAEAEKILQIKRAEGEAESKYLSGLGIARQRQAIVDGLRDSVLGFAVNVPGTTAKDVMDMVLVTQYFDTMKEIGASSKSSAVFIPHGPGAVRDVASQIRDGLLQGSQQ

SEQ ID NO: 119 - protein, 287 aa, Eutrema salsugineum
MGNLFCCVQVDQSTVAIKETFGKFEDVLEPGCHFLPWCLGSQVAGYLSLRLQQLDVRCETKTKDNVFVNVVASIQYRAIANQANDAFYKLSNTKGQIQAYVFDVIRASVPKLLLDDVFEQKDEIAKAVEEELEKAMSAYGFEIVQTLIVDIEPDEHVKRAMNEINAAARMRLAANEKAEAEKILQIKRAEGEAEAKYLSGLGIARQRQAIVDGLRDSVLGFSVNVPGTTAKDVMDMVLVTQYFDTMKEIGASSKSSAVFIPHGPGAVRDVATQIRDGLLQGSFADQS

SEQ ID NO: 120 - protein, 287 aa, Oryza brachyantha
MGNLFCCVQVDQSTVAIRETFGKFDSVLEPGCHCLPWFIGKRIAGHLTLRLQQLDVRCETKTKDNVFVNVVASIQYRALAGKANDAFYKLSNTRSQIQAYVFDVIRASVPKLNLDDAFEQKNDIAKAVEDELEKAMSAYGFEIVQTLIVDIEPDEHVKRAMNEINAAARLRVAANEKAEAEKIVQIKRAEGEAEAKYLSGLGIARQRQAIVDGLRDSVLGFSVNVPGTTAKDVMDMVLITQYFDTMKEIGASSKASSVFIPHGPGAVRDIATQIRDGLLQGHAAAAI

SEQ ID NO: 121 - protein, 286 aa, Cucumis sativus
MGNLFCCVKVDQSTVAIRETFGKFDEVVEPGCHCLPWFLGAQIAGTLSLRLQQLDVRCETKTKDNVFVNVVASIQYRALADKANDAFYKLSNTRSQIQAYVFDVIRASVPKLNLDDAFEQKNDIAKAVEDELEKAMSAYGFEIVQTLIVDIEPDEHVKRAMNEINAAARLRVAANEKAEAEKILQIKRAEGEAEAKYLSGLGIARQRQAIVDGLRDSVLGFSVNVPGTTAKDVMDMVLVTQYFDTMKEIGAASKSTSVFIPHGPGAVRDVASQIRDGLLQGAATSH

SEQ ID NO: 122 - protein, 285 aa, Solanum lycopersicum
MGNLFCCVQVDQSTVAIKEQFGKYQDVLEPGCHCVPWFLGSQLAGHLSLRVQQLDVRCETKTKDNVFVNVVASIQYRAIADKANEAFYKLSNTKGQIQAYVFDVIRASVPKLILDDVFEQKNEIAKAVEEELEKAMSAYGYEIVQTLIVDIVPDEHVKRAMNEINAAARLRVAANEKAEAEKILQIKRAEGEAESKYLSGLGIARQRQAIVDGLRDSVLGFSVNVPGTTAKDVMDMVLVTQYFDTMKEIGASSKSSAVFIPHGPGAVKEVAQQIRDGLLQATVGH

SEQ ID NO: 123 - protein, 291 aa, Solanum lycopersicum
MLCFQNMGNLFCCVQVDQSTVAIKEQFGKYQDVLEPGCHCVPWFLGSQLAGHLSLRVQQLDVRCETKTKDNVFVNVVASIQYRAIADKANEAFYKLSNTKGQIQAYVFDVIRASVPKLILDDVFEQKNEIAKAVEEELEKAMSAYGYEIVQTLIVDIVPDEHVKRAMNEINAAARLRVAANEKAEAEKILQIKRAEGEAESKYLSGLGIARQRQAIVDGLRDSVLGFSVNVPGTTAKDVMDMVLVTQYFDTMKEIGASSKSSAVFIPHGPGAVKEVAQQIRDGLLQATVGH

SEQ ID NO: 124 - protein, 285 aa, Solanum tuberosum
MGNLFCCVQVDQSTVAIKEQFGKYQDVLEPGCHCVPWFLGSQLAGHLSLRVQQLDVRCETKTKDNVFVNVVASIQYRAIADKANEAFYKLSNTKGQIQAYVFDVIRASVPKLILDNVFEQKNEIAKAVEEELEKAMSAYGYEIVQTLIVDIVPDEHVKRAMNEINAAARLRVAANEKAEAEKILQIKRAEGEAEAKYLSGLGIARQRQAIVDGLRDSVLGFSVNVPGTTAKDVMDMVLVTQYFDTMKEIGASSKSSAVFIPHGPGAVKDVAQQIRDGLLQASVGH

SEQ ID NO: 125 - protein, 285 aa, Solanum lycopersicum
MGNLLGCVQVDQSTVAITEQFGKYQDVLQPGCHCLPWCFGFQLAGHLSLRVQQLDVRCETKTKDNVFVNVVASIQYRALADKANDAFYKLSNTKGQIQAYVFDVIRASVPKLNLDDVFEQKNEIAKAVEDELEKAMSAYGYEIVQTLIVDIEPDENVKRAMNEINAAARLRVAANEKAEAEKILQIKRAEGEAESKYLSGLGIARQRQAIVDGLRDSVLGFSVNVPGTSAKDVMDMVLLTQYFDTMKEIGASSKSSAVFLPHGPGAVRDVASQIRDGLLQASVNH

SEQ ID NO: 126 - protein, 284 aa, Cicer arietinum
MGNLFCCVQVDQSTVAMKEGFGRFEEVLQPGCHCMPWVLGKRIAGHLSLRLQQLDIRCETKTKDNVFVNVVASIQYRALVNKANDAFYKLSNTRSQIQAYVFDVIRGTVPKLNLDDAFEQKNEIAKAVEDELEKAMSAYGYEIVQTLIVDIEPDEHVKRAMNEINAAARLRMAAKDKAEAEKILQIKRAEGEAESKYLSGLGIARQRQAIVDGLRDSVLGFSVNVPGTTAKDVMDMVLVTQYFDTMKEIGAASKASAVFIPHGPGAVRDVASQIRDGLLQGSYH

SEQ ID NO: 127 - protein, 287 aa, Setaria italica
MGNLCCCVQVDQSTVAIREQFGKFDSVLEPGCHCMPWFIGKRVAGHLTLRLQQLDVRCETKTKDNVFVNVVASIQYRALAGKASDAFYKLSNTRSQIQAYVFDVIRASVPKLILDDAFEQKDEIAKAVEEELEKAMSAYGFEIVQTLIVDIEPDEHVKRAMNEINAAARLRVAANEKAEAEKIVQIKRAEGEAEAKYLSGLGIARQRQAIVDGLRDSVLGFSVNVPGTTAKDVMDMVLITQYFDTMKEIGASSKASSVFIPHGPGAVRDIATQIRDGLLQGSAVSHH

SEQ ID NO: 128 - protein, 288 aa, Oryza sativa
MGNLFCCVQVDQSTVAIREQFGKFDAVLEPGCHCLPWFAGKRIAGHLTLRLQQLDVRCETKTKDNVFVNVVASIQYRALAGKANDAFYKLSNTRSQIQAYVFDVIRASVPKLNLDDAFEQKNDIAKAVEDELEKAMSAYGFEIVQTLIVDIEPDEHVKRAMNEINAAARLRVAANEKAEAEKIVQIKRAEGEAEAKYLSGLGIARQRQAIVDGLRDSVLGFSVNVPGTTAKDVMDMVLITQYFDTMKEIGASSKASSVFIPHGPGAVRDIATQIRDGLLQGQATTTSH

SEQ ID NO: 129 - protein, 357 aa, Brachypodium distachyon
MGWKLDGAASRGLFHKLGEKDGRVGPENRVNKAEKAEKKIPRSKHFVGAALLPDSALLAGTTRRDLKVSIMGNLCCCVQVDQSTVAIREQFGRFDSVLEPGCHCLPWMIGKRIVGHLTLRLQQLDVRCETKTKDNVFVTVVASIQYRPLAGKESDAFYKLTNTRSQIQAYVFDVIRASVPKLNLDDAFEQKNDIAKAVEDELEKAMSAYGFEIVQTLIVDIEPDEHVKRAMNEINAAARMRVAANEKAEAEKIVQIKRAEGEAEAKYLSGLGIARQRQAIVDGLRDSVLGFSVNVPGTTAKDVMDMVLITQYFDTMKEIGASSKSSAVFIPHGPGAVRDIATQIRDGLLQGQSAAHQ

SEQ ID NO: 130 - protein, 286 aa, Vitis vinifera
MGQAFCCIQVDQSNVAIKEQFGKFDEVLEPGCHCLPWCFGSQLAGHLSLRVQQLDVRCETKTKDNVFVTVVASIQYRALAEKASDAFYKLSNTRAQIQAYVFDVIRASVPKLDLDSTFEQKNEIAKAVEEELEKAMSAYGFEIVQTLIVDIEPDEHVKRAMNEINAASRMRLAATEKAEAEKILQIKRAEGDAESKYLAGLGIARQRQAIVDGLRDSVLAFSVNVPGTSSKDVMDMILVTQYFDTLKDIGASSKASSVFIPHGPGAVGDIASQIRVGLLQAETAKH

SEQ ID NO: 131 - protein, 284 aa, Zea mays
MGQILGLVQVDQSTVAIKENFGKFSEVLEPGCHFLPWCIGQQIAGYLSLRVRQLDVRCETKTKDNVFVTVVASVQYRALADKASDAFYKLSNTREQIQSYVFDVIRATVPKLGLDDAFEQKNEIAKAVEEELEKAMSTYGYQIVQTLIVDIEPDDRVKRAMNEINAAARMRVAASEKAEAEKILQIKKAEGEAESKYLAGVGIARQRQAIVDGLRDSVLAFSENVPGTTAKDIMDMVLVTQYFDTMREIGASSKSSSVFIPHGPGAVKDVSAQIRDGLLQANLH

SEQ ID NO: 132 - protein, 283 aa, Setaria italica
MGQVLGLVQVDQSTVAIKETFGKFSEVLEPGCHFLPWCIGQQVAGYLSLRVRQLDVRCETKTKDNVFVTVVASVQYRALADKASDAFYKLSNTREQIQSYVFDVIRATVPKLELDDAFEQKNDIAKAVEEELEKAMSTYGYEIVQTLIVDIEPDDRVKRAMNEINAAARMRLAASEKAEAEKIIQIKKAEGEAESKYLAGVGIARQRQAIVDGLRDSVLAFSENVPGTTAKDIMDMVLVTQYFDTMKEIGASSKSSSVFIPHGPGAVKDVAAQIRDGLLQASQ

SEQ ID NO: 133 - protein, 286 aa, Fragaria vesca
MGQCLGCVQVDQSTVAIRETFGKFDDVLEPGCHCLPWCLGSAIAGHLSLRLQQLDVRCETKTKDNVFVTVVASIQYRALADKASDAFYKLSNTRGQIQSYVFDVIRASVPKLDLDSTFEQKNDIAKAVEEELEKAMSHYGFEIVQTLIVDIEPDEHVKRAMNEINAAARMRLAATEKAEAEKILQIKRAEGEAESKYLSGLGIARQRQAIVDGLRDSVLAFSENVPGTSSKDVMDMVLVTQYFDTMKEIGASSKSNSVFIPHGPGAVKDIASQIRDGLLQGNSSRS

SEQ ID NO: 134 - protein, 285 aa, Populus trichocarpa
MGNLCCCVQVDQSSVAIKETFGKFEAVLDPGCHCLPWFLGSQLAGHLSLRLQQLDVRCETKTKDNVFVNVVASIQYRALADKASDAFYKLTNTRTQIQAYVFDVIRASVPKLNLDDVFEQKNEIAKAVEDELGKAMSAYGYEIVQTLIVDIEPDEHVKRAMNEINAAARLRLAANEKAEAEKILQIKRAEGEAESKYLSGLGIARQRQAIVDGLRDSVLGFSENVPGTSAKDVMDMVLVTQYFDTMKEIGAASKSSAVFIPHGPGAIRDVATQIRDGLLQASAHK

SEQ ID NO: 135 - protein, 285 aa, Populus trichocarpa
MGQAFGCLQVDQSNVAIKEQFGKFVDVLEPGCHCLPWCFGYQVAGGLSLRVQQLDVRCETKTKDNVFVTVVASIQYRAMAEKASDAFYKLSNTKAQIQAYVFDVIRASVPKLLLDDTFEQKNDIAKAVENELEKAMSAYGYEIVQTLIVDIEPDINVKRAMNEINAAARLRVAANEKAEAEKILQIKRAEGEAESKYLSGLGIARQRQAIVDGLRDSVLAFSENVPGTSAKDVMDMVLVTQYFDTMKEIGASSKSSSVFIPHGPGAVRDITSQIRDGLLQGNSAQ

SEQ ID NO: 136 - protein, 284 aa, Oryza sativa
MGQALGLVQVDQSTVAIKESFGKFDEVLEPGCHFLPWCIGKQIAGYLSLRVQQLDVRCETKTKDNVFVNVVASVQYRALAEKASDAFYRLSNTREQIQSYVFDVIRASVPKMNLDDAFEQKNEIAKAVEDELEKAMSMYGYEIVQTLIVDIEPDEHVKRAMNEINAAARLRVAANEKAEAEKILQIKRAEGDAESKYLAGLGIARQRQAIVDGLRDSVLAFSENVPGTSAKDVMDMVLVTQYFDTMKEIGASSKSSSVFIPHGPGAVKDIAAQIRDGQLQAKLI

SEQ ID NO: 137 - protein, 284 aa, Sorghum bicolor
MGQALGLVQVDQSTVAIKENFGKFSEVLEPGCHFLPWCIGQQIAGYLSLRVRQLDVRCETKTKDNVFVTVVASVQYRALAEKASDAFYKLSNTREQIQSYVFDVIRATVPKLDLDDAFEQKNDIAKAVEEELEKAMSMYGYEIVQTLIVDIEPDDRVKRAMNEINAAARMRVAASEKAEAEKILQIKKAEGEAESKYLAGVGIARQRQAIVDGLRDSVLAFSENVPGTTAKDIMDMVLVTQYFDTMKEIGASSKSSSVFIPHGPGAVKDVAAQIRDGLLQAKLQ

SEQ ID NO: 138 - protein, 284 aa, Brachypodium distachyon
MGQVLGLVQIDQSTVAIKESFGKFDAILEPGCHCLPWCFGKQIAGYLSLRVQQLDVRCETKTKDNVFVNVVASVQYRALADKASDAFYRLSNTREQIQSYVFDVIRASVPKMNLDDVFEQKNEIAKAVEDELEKAMSAYGYEIVQTLIVDIEPDEHVKRAMNEINAAARMRLAATEKAEAEKILQIKRAEGDAESKYLAGLGIARQRQAIVDGLRDSVLAFSENVPGTSSKDVMDMVLVTQYFDTMKDIGATSKTSAVFIPHGPGAVKDIASQIRDGQLQAKMG

SEQ ID NO: 139 - protein, 284 aa, Oryza brachyantha
MGQALGLIQVDQSTVAIKESFGKFDEVLEPGCHFLPWCIGKQIAGYLSLRVQQLDVRCETKTKDNVFVNVVASVQYRALADKASDAFYKLSNTREQIQSYVFDVIRASVPKMNLDDAFEQKNEIAKAVEDELEKAMSMYGYEIVQTLIVDIEPDEHVKRAMNEINAAARLRVAANEKAEAEKILQIKRAEGDAESKYLAGLGIARQRQAIVDGLRDSVLAFSENVPGTSAKDVMDMVLVTQYFDTMKEIGASSKSSSVFIPHGPGAVKDIAAQIRDGQLQAKFV

SEQ ID NO: 140 - protein, 284 aa, Setaria italica
MGQALGLIQVDQSTVAIKESFGKFDEILGPGCHFLPWCIGKQIAGYLSLRVQQLDVRCETKTKDNVFVNVVASVQYRALADKASDAFYRLTNTREQIQSYVFDVIRASVPKMNLDDTFEQKNEIAKAVEDELEKAMSMYGYEIVQTLIVDIEPDEHVKRAMNEINAAARLRVAANEKAEAEKILQIKRAEGDAESKYLAGLGIARQRQAIVDGLRDSVLAFSENVPGTSAKDVMDMVLVTQYFDTMKEIGASSKSSSVFIPHGPGAVKDIAAQIRDGSLQAKLV

SEQ ID NO: 141 - protein, 286 aa, Glycine max
MGQVLGCVQVDQSSLAIKEVFGKYDDVLEPGCHCVPWCFGSRVAGALSLRVKQLDVRCETKTKDNVFVTVVASIQYRALAEKAVDAYYKLSNTRSQIQSYVFDVIRASVPKMELDATFEQKNEIAKAVEEELEKAMSAYGYEIVQTLIVDIEPDEHVKRAMNEINAAARLRVAANEKAEAEKILQIKRAEGDAESKYLAGLGIARQRQAIVDGLRDSVLAFSENVPGTTSKDIMDMVLMTQYFDTMKDIGASSKSNAVFIPHGPGAVQDVASQIRNGLLQGNATQS

SEQ ID NO: 142 - protein, 285 aa, Ricinus communis
MGQALGCVQVDQSTVAIKETFGKFDDVLEPGCHCLPWCLGSQLAGHLSLRVQQLDVRCETKTKDNVFVTVVASIQYRALAEKAADAFYKLSNTRAQIQAYVFDVIRASVPKLDLDSAFEQKNDIAKAVENELEKAMSHYGFEIVQTLIVDIEPDEHVKRAMNEINAAARMRVAASEKAEAEKILQIKRAEGDAESKYLAGLGIARQRQAIVDGLRDSVLAFSENVPGTSSKDVMDMVLVTQYFDTMKEIGASSKSSSVFIPHGPGAVRDIATQIRDGLLQANSTR

SEQ ID NO: 143 - protein, 284 aa, Cucumis sativus
MGQALGCIQVDQSTVAIRETFGRFDDVLQPGCHCLPWCLGSQIAGHLSLRLQQLDVRCETKTKDNVFVTVVASIQYRALADKASDAFYKLSNTREQIQAYVFDVIRASVPKLDLDSTFEQKNDIAKAVEDELEKAMSAYGYEIVQTLIVDIEPDEHVKRAMNEINAAARLRVAATEKAEAEKILQIKRAEGDAESKYLAGLGIARQRQAIVDGLRDSVLAFAENVPGTTSKDVMDMVLVTQYFDTMKEIGASSKSNSVFIPHGPGAVKDIASQIRDGLLQASQT

SEQ ID NO: 144 - protein, 299 aa, Medicago truncatula
MLLHLSLATTEKKMGNLVCCVQVDQSQVAMKEGFGKFEKVLQPGCHCMPWFLGKRIAGHLSLRVQQLDIKCETKTKDNVFVNVVASIQYRALADKANDAFYKLSNTRNQIQAYVFDVIRASVPKLNLDDTFEQKNEIAKAVEEELEKAMSAYGYEIVQTLITDIEPDVHVKRAMNEINAAARMRLAAKEKAEAEKILQIKRAEGEAESKYLSGLGIARQRQAIVDGLRDSVIGFSVNVPGTTAKDVMDMVLVTQYFDTMKEIGAASKSSAVFIPHGPGAVRDVASQIRDGLLQGSLSHQ

SEQ ID NO: 145 - protein, 299 aa, Medicago truncatula
MGNIVCCVQVDQSQVAMKEGFGKFEKVLHPGCHCMPWFLGKRIAGHLSLRVQQLDIKCETKTKAGLLLSKTSIIPNLHDNVFVNVVASIQYRALANKANDAFYKLSNTRGQIQAYVFDVIRASVPKLNLDDTFEQKNEIAKAVEEEREKAMSAYGYEIVQTLITDIEPDQHVKTAMNEINAAARMRIAANEKAEAEKILQIKRAEGEAESKYLSGMGIARQRQAIVDGLRDSVIGFSENVPGPSAKDVMDMVLVTQYFDTMKEIGAASKSSAVFIPHGPGAVRDVASQICDGLLQGSHQ

SEQ ID NO: 146 - protein, 286 aa, Solanum tuberosum
MGQTLGCIQVDQSTVAVKEQFGKFDQVLEPGCHCLPWCLGYQVAGSLSLRVQQLDVRCETKTKDNVFVTVVASIQYRALAENAADAFYKLSNTKGQIQSYVFDVIRASVPRMGLDAAFEQKNEIAKAVEEELEKAMSAYGYEIVQTLIVDIEPDIHVKRAMNEINAAARLRVAANEKAEAEKILQIKKAEGEAESKYLSGLGIARQRQAIVDGLRDSVLAFSENVPGTSPRDVMDMVLVTQYFDTMKEIGASSKSSAVFIPHGPGAVKDVASQIREGLLQAESIQH

SEQ ID NO: 147 - protein, 284 aa, Brachypodium distachyon
MAGILGLVQVDQSTVAIKETFGKFSEVLEPGCHFLPWCIGQRIVGYLSLRVKQLDVRCETKTKDNVFVTVVASIQYRALADKASDAFYKLSNTKQQIQSYVFDVIRATVPKLGLDDAFEQKDDIAKAVEEELEKAMSMYGYEIVQTLIVDIEPDVHVKRAMNEINAAARMRSATNEKAEAEKIIQIKRAEGEAESKYLAGVGIARQRQAIVDGLRDSVLAFSENVPGTTAKDVMDMVLVTQYFDTMKEIGASSKSSSVFIPHGPGAVKDVASQIRDGLLQSNIV

SEQ ID NO: 148 - protein, 284 aa, Glycine max
MGNLFCCVKVDQSTVAMREGFGRFEKVLQPGCHCMPWFLGKQLAGHLSLRLQQLDLRCETKTKDNVFVNVVASIQYRALAEKANDAFYKLSNTKTQIQAYVFDVIRASVPKLNLDDAFEQKSEIARAVEEELEKAMSAYGYEIVQTLIVDIDPDVHVKRAMNEINAAARLRLAANEKAEAEKILLIKRAEGEAESKYLSGLGIARQRQAIVDGLRDSVLGFSVNVPGTSARDVMDMVLVTQYFDTMKDIGAASKSSAVFIPHGPGAVRDVASQIRDGLLQASHQ

SEQ ID NO: 149 - protein, 286 aa, Selaginella moellendorffii
MGQMCCCFQVPQSRVAIKERWGKFDEVLDPGCHCVPWIFGSNITGSLNLRIQQLDVRCETKTKDNVFVTVVASVQYAVVQADAMDAYYKLSNPREQIQAYVFDVVRACVPKMILDDVFEQKNEVAKSVEDELEKAMAAYGYRIVQTLIVDVEPDKTVRNAMNEINAAARLRVAANEKAEAEKILQVKRAEAEAESKYLSGVGVARQRQAIVDGLRESVLAFSHNVPGTSAKDVMDMVLLTQYFDTMKEIGATSKSSTVFLPHGPGAVRDVAEQIRNGMLQAEAFTQ

SEQ ID NO: 150 - protein, 346 aa, Medicago truncatula
MIITPNMGFKDRCELLFFKSLIIGPPTASLVNRPYTHTHTQFFCCFAERQPNQTTNVEKKMGNLVCCVQVDQSQVAMKEGFGKFEKVLQPGCHCMPWFLGKRIAGHLSLRVQQLDIKCETKTKDNVFVNVVASIQYRALADKANDAFYKLSNTRNQIQAYVFDVIRASVPKLNLDDTFEQKNEIAKAVEEELEKAMSAYGYEIVQTLITDIEPDVHVKRAMNEINAAARMRLAAKEKAEAEKILQIKRAEGEAESKYLSGLGIARQRQAIVDGLRDSVIGFSVNVPGTTAKDVMDMVLVTQYFDTMKEIGAASKSSAVFIPHGPGAVRDVASQIRDGLLQGSLSHQ

SEQ ID NO: 151 - protein, 358 aa, Medicago truncatula
MIITPNMGFKDRCELLFFKSLIIGPPTASLVNRPYTHTHTQFFCCFAERQPNQTTNVTLPSQLYFEHCSEKKMGNLVCCVQVDQSQVAMKEGFGKFEKVLQPGCHCMPWFLGKRIAGHLSLRVQQLDIKCETKTKDNVFVNVVASIQYRALADKANDAFYKLSNTRNQIQAYVFDVIRASVPKLNLDDTFEQKNEIAKAVEEELEKAMSAYGYEIVQTLITDIEPDVHVKRAMNEINAAARMRLAAKEKAEAEKILQIKRAEGEAESKYLSGLGIARQRQAIVDGLRDSVIGFSVNVPGTTAKDVMDMVLVTQYFDTMKEIGAASKSSAVFIPHGPGAVRDVASQIRDGLLQGSLSHQ

SEQ ID NO: 152 - protein, 285 aa, Oryza brachyantha
MGQCLGLVQIDQSNVAIKETFGKFSGVLEPGCHCLPWCFGQQIAGYLSLRVKQLDVRCDTKTKDNVFVTVVASVQYRALADKACDAFYKLSNTKGQIQSYVFDVIRATVPKLNLDDAFEQKNDIAKAVEDELEKAMSTYGYEIVQTLIIDIEPDVHVKRAMNEINAAARLRVAANEKAEAEKILQIKKAEGEAESKYLAGVGVARQRQAIVDGLRDSVLAFSENVPGTSAKDIMDMVLVTQYFDTMKEIGANSKSSSVFIPHGPGAVRDVAAQIRDGLLQANAQQ

SEQ ID NO: 153 - protein, 284 aa, Medicago truncatula
MGNLLCCVQVDQSTVAMREGFGKFEEVLQPGCHCMPWFLGKRIAGHLSLRLQQLDIKCETKTKDNVFVNVVASIQYRALADKANEAFYKLSNTRGQIQAYVFDVIRASVPKLYLDDAFEQKNEIAKAVEEELEKAMSAYGYEIVQTLITDIEPDEHVKRAMNEINAAARMRLAAKEKAEAEKILQIKRAEGEAESKYLSGMGIARQRQAIVDGLRDSVIGFSVNVPGTSAKDVMDMVLVTQYFDTLKEIGAASKSSAVFIPHGPGAVRDVASQIRDGLLQGSHQ

SEQ ID NO: 154 - protein, 289 aa, Glycine max
MGQMFSMFGFVQVEQSSVAIKEVFGKYNDVLDPGCHCVPWYFGNRVAGALSLRIQQLDVRCETKTKDNVFVTVVASIQYRALAEKASDAYYKLSNTRAQIQSYVFDVIRASVPKMELDATFEQKTDIAKTVEEELEKAMSAYGYEIVQTLIVDIEPDERVKRAMNEINAAARLRVASNEKAEAEKIVQIKRAEGDAESKYLAGLGVARQRQAIVDGLRDSVLAFSENVPGTTSKDIMDMVLMTQYFDTMKDIGASSKSNAVFIPHGPGSVHDVASQIRDGLQQGNATKS

SEQ ID NO: 155 - protein, 289 aa, Glycine max
MGQMFSMCGFVQVEQSSVVIKEVFGKYDDVLDPGCHCVPWYFGNRVAGALSLRIQQLDVRCETKTKDNVFVTVVASIQYRALAEKASDAYYKLTNTKAQIQSYVFDVIRASVPKMELDATFEQKNDIAKTVEEELEKAMSAYGYEIVQTLIVDIEPDERVKRAMNEINAAARLRVASNEKAEAEKILQIKRAEGDAESKYLAGLGVARQRQAIVDGLRDSVIAFSENVPGTTSKDILDMVLMTQYFDTMKDIGASSKSNAVFIPHGPGSVHDVASQIRDGLLQGNATKS

SEQ ID NO: 156 - protein, 317 aa, Oryza sativa
MELSKPQSKSKLQPLTNHLLSVFTSANLNSSSDSPLPRSKIDQSTVAIKENFGKFSEVLEPGCHFLPWCIGQQIAGYLSLRVKQLDVRCETKTKDNVFVTVVASVQYRALADKASDAFYKLSNTREQIQSYVFDVIRATVPKLNLDDAFEQKNDIAKAVEDELEKAMSAYGYEIVQTLIIDIEPDVHVKRAMNEINAGKLRVAANEKAEAEKILQIKKAEGEAESKYLAGVGIARQRQAIVDGLRDSVLAFSENVPGTTAKDIMDMVLVTQYFDTMKEIGASSKSTSVFIPHGPGAVKDVAAQIRDGLLQANAERND

SEQ ID NO: 157 - protein, 287 aa, Glycine max
MGQVLGCVQVKQSTVAVKECFGKYDDVLQPGCHFVPWCLGCGVAGVLSTRVMQLSLRCETKTKDNVFVNVVASIQYRALAEKASDAYYKLTNTKAQIQSYVFDVIRATVPKMELDAVFEQKNTIAKAVDEELGKAMSAYGYEIVQTLIVDIVPDEHVKKAMNEINAAARLRVATNDKAEAEKIMQIKRAEGDAESKYLAGLGVSRQRQAIVDGLRDSVLAFSGNVPGTSSKDIMDMVLMTQYFDTMKEIGASSKSNAVFIPHGPGAVSDVASQVRNGLLQGNATTES

SEQ ID NO: 158 - protein, 286 aa, Glycine max
MGQAFGCYQVDQSNVAIKEHFGKFDDVLEPGCHCLPWCLGYQIAGSLSLRVQQLDVRCETKTKDNVFVNVVASVQYRAVSEKASDAFYRLTNTREQIQSYVFDVIRASVPKLELDSVFEQKNDIAKAVEEELVKAMSTYGFEIVQTLIVDIEPDVNVKRAMNEINTAARLRLAANEKAEAEKILQIKKAEGEAESKYLSGLGIARQRQAIVDGLRDSVLAFSENVPGTSAKDVMDMVLVTQYFDTMKEIGASSRSSSVFIPHGPGAVKDIAVQIRDGLLQATASRN

SEQ ID NO: 159 - protein, 284 aa, Zea mays
MGQALGLIQVDQSTVAIKETFGKFDEVLEPGCHFLPWCIGKQIAGYLSLRVQQLDVRCETKTKDNVFVNVVASVQYRALADKASDAFYRLSNTREQIQSYVFDVIRASVPKMNLDDAFEQKNEIAKAVENELEKAMSMYGYEIVQTLIVDIEPDEHVKRAMNEINAAARLRLAASEKAEAEKILQIKRAEGDAESKYLAGLGIARQRQAIVDGLRDSVLAFSENVPGTSAKDVMDMVLVTQYFDTMKEIGASSKSSSVFIPHGPGAVRDIAAQIRDGQLQASLL

SEQ ID NO: 160 - protein, 252 aa, Amborella trichopoda
MPWFLGSQLAGHLTLRLQQLDVRCETKTKDNVFVTVVASIQYRALADKASDAFYKLSNTRTQIQAYVFDVIRASVPKLDLDDVFEQKNDIAKAVEDELEKAMSAYGYQIVQTLIVDIEPDEHVKRAMNEINAASRMRLATKEKAEAEKIIQIKRAEGEAESKYLAGLGIARQRQAIVDGLRDSVLGFSVNVPGTTAKDVMDMVLVTQYFDTMKEIGATSKSSAVFIPHGPGAVRDVAAQIRDGLLQSQVHHE

SEQ ID NO: 161 - protein, 287 aa, Zea mays
MGNLCCCVQVDQSTVAIREQFGKFDSVLEPGCHCMPWFAGKRVAGQLTLRLQQLDVRCETKTKDNVFVNVVASIQYRALADKASDAFYKLSNTRSQIQAYVFDVIRASVPKLHLDDAFEQKDEIARAVEEELEKAMSAYGFEIVQTLIVDIEPDEHVKRAMNEINAAARLRAAANEKAEAEKIVQIKRAEGEAEAKYLSGLGIARQRQAIVDGLRDSVLGFSVNVPGTTAKDVMDMVLITQYFDTMKEIGASSKASSVFIPHGPGAVRDIATQIRDGLLQGSSVAKH

SEQ ID NO: 162 - protein, 285 aa, Citrus clementina
MGQALGCIQVDQSTVAIRETFGKFDEVLEPGCHCLPWCLGSQVAGQLSLRVQQLDVRCETKTKDNVFVNVVASVQYRALAEKASDAFYKLSNTRSQIQAYVFDVIRASVPKLDLDATFEQKNDIAKAVEEELEKAMSHYGYEIVQTLIVDIEPDEHVKRAMNEINAAARLRLAANEKAEAEKILQIKRAEGEAESKYLAGLGIARQRQAIVDGLRDSVLAFSENVPGTSSKDVMDMVLVTQYFDTMKEIGASSKTNSVFIPHGPGAVKDIASQIREGLLQANATM

SEQ ID NO: 163 - protein, 286 aa, Citrus clementina
MGQTLGCIQVEQSKVVIREQFGKFDHVLEPGCQCLPWCLGYKVAGRLSLRVQQLDVKCETKTKDNVFVNVVASVQYRALADKAYDAFYKLSNTRGQIQAYVFDVIRASVPKLNLDAAFEQKNEIAKAVEEELEKAMSHYGYEIVQTLIVDIEPDVHVKRAMNEINAAARLRLAANEKAEAEKILQIKRAEGEAEAKYLSGLGIARQRQAIVDGLRDSVLAFSENVPGTTSKDVMDMVLVTQYFDTMKEIGASSKSSSVFIPHGPGAVKDIATQIREGLLQANQVQK

SEQ ID NO: 164 - protein, 306 aa, Citrus clementina
MKSRNCRGLVDDSSNELYNQKMGQALGCIQVDQSTVAIRETFGKFDEVLEPGCHCLPWCLGSQVAGQLSLRVQQLDVRCETKTKDNVFVNVVASVQYRALAEKASDAFYKLSNTRSQIQAYVFDVIRASVPKLDLDATFEQKNDIAKAVEEELEKAMSHYGYEIVQTLIVDIEPDEHVKRAMNEINAAARLRLAANEKAEAEKILQIKRAEGEAESKYLAGLGIARQRQAIVDGLRDSVLAFSENVPGTSSKDVMDMVLVTQYFDTMKEIGASSKTNSVFIPHGPGAVKDIASQIREGLLQANATM

SEQ ID NO: 165 - protein, 270 aa, Medicago truncatula
MKEGFGKFEKVLQPGCHCMPWFLGKRIAGHLSLRVQQLDIKCETKTKDNVFVNVVASIQYRALADKANDAFYKLSNTRNQIQAYVFDVIRASVPKLNLDDTFEQKNEIAKAVEEELEKAMSAYGYEIVQTLITDIEPDVHVKRAMNEINAAARMRLAAKEKAEAEKILQIKRAEGEAESKYLSGLGIARQRQAIVDGLRDSVIGFSVNVPGTTAKDVMDMVLVTQYFDTMKEIGAASKSSAVFIPHGPGAVRDVASQIRDGLLQGSLSHQ

SEQ ID NO: 166 - protein, 286 aa, Glycine max
MGQALGCYQVDQSNVAIKEHFGKFDDVLEPGCHCLPWCLGYQIAGSLSLRVQQLDVRCETKTKDNVFVTVVASVQYRAVSEKASDAFYRLTNTREQIQSYVFDVIRASVPKLELDSVFEQKNDIAKAVEEELEKAMSTYGFEIVQTLIVDIEPDVNVKRAMNEINAAARLRLAANEKAEAEKILQIKKAEGEAESKYLSGLGIARQRQAIVDGLRDSVLAFSENVPGTTAKDVMDMVLVTQYFDTMKEIGASSKSSSVFIPHGPGAVKDIAMQIRDGLLQATTSQN

SEQ ID NO: 167 - protein, 286 aa, Citrus sinensis
MGQTLGCIQVEQSKVVIREQFGKFDHVLEPRCQCLPWCLGYKVAGRLSLRVQQLDVKCETKTKDNVFVNVVASVQYRALADKAYDAFYKLSNTRGQIQAYVFDVIRASVPKLNLDAAFEQKNEIAKAVEEELEKAMSHYGYEIVQTLIVDIEPDVHVKRAMNEINAAARLRLAANEKAEAEKILQIKQAEGEAEAKYLSGLGIARQRQAIVDGLRDSVLAFSENVPGTTSKDVMDMVLVTQYFDTMKEIGASSKSSSVFIPHGPGAVKDIATQIREGLLQANQVQK

SEQ ID NO: 168 - protein, 286 aa, Glycine max
MGQAFGCYQVDQSNVAIKEHFGKFDDVLEPGCHCLPWCLGYQIAGSLSLRVQQLDVRCETKTKDNVFVNVVASVQYRAVSEKASDAFYRLTNTREQIQSYVFDVIRASVPKLELDSVFEQKNDIAKAVEEELEKAMSTYGFEIVQTLIVDIEPDVNVKRAMNEINAAARLRLAANEKAEAEKILQIKKAEGEAESKYLSGLGIARQRQAIVDGLRDSVLAFSENVPGTSAKDVMDMVLVTQYFDTMKEIGASSRSSSVFIPHGPGAVKDIAVQIRDGLLQATASQN

SEQ ID NO: 169 - protein, 286 aa, Arabidopsis thaliana
MGQALGCIQVDQSNVAIKETFGKFDEVLEPGCHCLPWCLGSQVAGHLSLRVQQLDVRCETKTKDNVFVTVVASIQYRALAESAQDAFYKLSNTRNQIQAYVFDVIRASVPKLDLDSTFEQKNDIAKTVETELEKAMSHYGYEIVQTLIVDIEPDVHVKRAMNEINAASRMREAASEKAEAEKILQIKRAEGEAESKYLSGMGIARQRQAIVDGLRNSVLAFSESVPGTSSKDVMDMVLVTQYFDTLKEIGASSKSNSVFIPHGPGAVRDIASQIRDGLLQGNSAAE

SEQ ID NO: 170 - protein, 286 aa, Eutrema salsugineum
MGQVLGCVQVDQSTVAIKETFGKFDDVLEPGCHCLPWCLGSQVAGHLSLRVQQLDVRCETKTKDNVFVTVVASIQYRALAESAQDAFYKLSNTRNQIQAYVFDVIRASVPKLDLDSTFEQKNDIAKTVESELEKAMSHYGYEIVQTLIVDIEPDVHVKRAMNEINAASRMREAASEKAEAEKILQIKRAEGEAESKYLSGMGIARQRQAIVDGLRNSVLAFSESVPGTSSKDVMDMVLVTQYFDTLKEIGASSKSNSVFIPHGPGAVKDIASQIRDGLLQGNAADS

SEQ ID NO: 171 - protein, 286 aa, Arabidopsis lyrata
MGQALGCIQVDQSNVAIKETFGKFDEVLEPGCHCLPWCLGSQVAGHLSLRVQQLDVRCETKTKDNVFVTVVASIQYRALAESAQDAFYKLSNTRNQIQAYVFDVIRASVPKLDLDSTFEQKNDIAKTVETELEKAMSHYGYEIVQTLIVDIEPDVHVKRAMNEINAASRMREAASEKAEAEKILQIKRAEGEAESKYLSGMGIARQRQAIVDGLRNSVLAFSESVPGTSSKDVMDMVLVTQYFDTLKEIGASSKSNSVFIPHGPGAVKDIASQIRDGLLQGNSVAE

SEQ ID NO: 172 - protein, 286 aa, Cicer arietinum
MGQALGCYQVDQSNVAIKEQFGRFVDVLEPGCHCLPWCLGYQIAGGLSLRVQQLDVKCETKTKDNVFVMVVASVQYRAVADKASDAFYRLTNTREQIQSYVFDVIRASVPKLELDAVFEQKNDIAKAVEDELEKAMSNYGYEIVQTLIVDVEPDVNVKRAMNEINAAARLRLAANDKAEAEKILQIKKAEGEAESKYLSGLGIARQRQAIVDGLRDSVLAFSENVPGTSAKDVMDMVLVTQYFDTMKEIGASSKSSSVFIPHGPGAVRDIAVQIRDGLLQGNAANL

SEQ ID NO: 173 - protein, 268 aa, Medicago truncatula
MREGFGKFEEVLQPGCHCMPWFLGKRIAGHLSLRLQQLDIKCETKTKDNVFVNVVASIQYRALADKANEAFYKLSNTRGQIQAYVFDVIRASVPKLYLDDAFEQKNEIAKAVEEELEKAMSAYGYEIVQTLITDIEPDEHVKRAMNEINAAARMRLAAKEKAEAEKILQIKRAEGEAESKYLSGMGIARQRQAIVDGLRDSVIGFSVNVPGTSAKDVMDMVLVTQYFDTLKEIGAASKSSAVFIPHGPGAVRDVASQIRDGLLQGSHQ

SEQ ID NO: 174 - protein, 286 aa, Glycine max
MGLALGCLQVEQSTVAIKEVFGKFDDVLEPGFHCVPWFFGTQVAGYLSLRVQQLDVRCETKTKDNVFVTVVASIQYRAMAERAVDAFYRLSNTREQIQAYVFDVIRACVPKMDLDSSFEQKKEIARAVEEELEKAMSAYGYEIVQTLIVDIEPDERVKRAMNEINAAARMREAANEKAEAEKILQIKKAEGDAESKYLSGLGIARQRQAIVDGLRDSVLAFSENVPGTTSKDVMDMVLVTQYFDTLKEIGASSKSNSIFVPHGPGTVRDIASQFRDGLLQGKVAQS

SEQ ID NO: 175 - protein, 286 aa, Capsella rubella
MGQALGCIQVDQSNVAIKETFGKFDEVLEPGCHCLPWCLGSQVAGHLSLRVQQLDVRCETKTKDNVFVTVVASIQYRAKPESAQDAFYKLSNTRNQIQAYVFDVIRASVPKLDLDSTFEQKNDIAKTVENELEKAMSHYGYEIVQTLIVDIEPDVHVKRAMNEINAASRMREAASEKAEAEKILQIKRAEGEAESKYLSGMGIARQRQAIVDGLRNSVLAFSESVPGTSSKDVMDMVLVTQYFDTLKEIGASSKSNAVFIPHGPGAVRDIASQIRDGLLQGNAAAE

SEQ ID NO: 176 - protein, 289 aa, Physcomitrella patens
MGQTLGLICVDQSTVAVKEQFGRYTGTIGPGCHCVPWCIGINVAGILSLRVQQLDVRCETKSRDNVFVTLVASVQYRCHTETAKDAFYKLTNPREQIKAYVFDVVRATVPKLLLDDVFEQKNEIANSVKEELEKSMKTYGYEIVQTLIVDIEPDETVKRAMNEINAAARMRLATLEKAEGEKILQVKRAEAEAESKYLSGVGIARQRQAIVDGLRESVMVFSDNVPGTTPREVMDMVLVTQYFDTMRDIGSHSKNSTVFIPHGVGGARDVADQIRNGLMQADAGKTHAH

SEQ ID NO: 177 - protein, 288 aa, Cicer arietinum
MGLALGCLQVEQSTVAIREVFGKYDDVLEPGCHCVPWCMGNQIAGYLSLRVQQLDVWCETKTKDNVFVTVVASVQYRALAEKVVDAFYRLTNTREQIQAYVFDVIRATVPKMELDSSFEQKNEIAKAVEEELGKAMSAYGYEIVQTLIVDIEPDEHVKKAMNEINAASRFRAAANEKAEAEKILLIKRAEGDAESKYLAGLGIARQRQAIVDGLRDSVLAFAENVPGTSSKDVMDMVLVTQYFDTLKEIGASSKSNSVFVPHGPGAVKDIASQVRDGLLQGSAACHEV

SEQ ID NO: 178 - protein, 254 aa, Ricinus communis
MGNLFCCITVDQSTVSIKERFGKFDEVLDPGCHCMPWILGSQLAGHLSLRLQQLDVRCETKTKDNVFVNVVASVQYRALADKASDAFYKLSNTRTQIQAYVFDVIRASVPKLNLDDVFEQKNEIAKAVEEELEKVTARLRVAANEKAEAEKIVQIKKAEGEAEAKYLSGVGIARQRQAIVDGLRDSVLGFSVNVPGTTAKDVLDMVLITQYFDTMKEIGATSKSSAVFIPHGPGAVNDVATQIRDGLLQATSNR

SEQ ID NO: 179 - protein, 289 aa, Physcomitrella patens
MGNTLGLYQVDQATVAVKERFGKFEGILTPGCHCTPWCIGVNVAGTLSLRVQQLDVRCETKTKDNVFVTVVASVQYRCHIETAEDAFYKLTNPREQIKSYVFDVVRASVPKMLLDDVFEQKNEIANNVKEELEKAMRTYGYEIVQTLIVDIEPDETVKRAMNEINAAARMRVAAVEKAEAEKILQVKRAEAEAESKYLSGMGIARQRQAIVNGLRESVMAFSDNVPGTSPAEVMDMVLVTQYFDTLRDIGASSKNSTVFIPHGVGGARDVADQIRNGLLQGDAGKAHAI

SEQ ID NO: 180 - protein, 286 aa, Selaginella moellendorffii
MACWVCVDQASVGILEKWGRFVRVLEPGFSCIVPCLGEFVAGTLSLKVQYLDVRCETKTKDNVFVSLDCSIQYRVVRGNADDAFYELQNPEQQIRSYVFDVIRASVPKLSLDDVFEQKSEIAKSVSEELEKVMSAYGYSIEQILIVDILPDAAVRRAMNEINAAQRMRMAAVEKGEAEKILQVKRAEGDAESKYLSGVGVARQRQAITDGLRESVLTFSQDVPGTSAKEVMEMVMITQYFDTLKDIGASSKTSAVFIPHGPAHVSDIAQQLRDGVLQANTAASLMD

SEQ ID NO: 181 - protein, 286 aa, Selaginella moellendorffii
MACWVCVDQASVGILEKWGRFVRVLEPGFSCIVPCLGEFVAGTLSLKVQYLDVRCETKTKDNVFVSLDCSIQYRVVRGNADDAFYELQNPEQQIRSYVFDVIRASVPKLSLDDVFEQKSEIAKSVSEELEKVMSAYGYSIEQILIVDILPDAAVRRAMNEINAAQRMRMAAVEKGEAEKILQVKRAEGDAESKYLSGVGVARQRQAITDGLRESVLTFSQDVPGTSAKEVMEMVMVTQYFDTLKDIGASSKTSAVFIPHGPAHVSDIAQQLRDGVLQANTAVSLMD

SEQ ID NO: 182 - protein, 272 aa, Solanum lycopersicum
MGQAFGCIQVDQSTIAIKENFGKYDDVLGPGFHFMPWCLGSQLAGYLSLRVQQLDITCESKTKDNVFVTLVASIQYRALADKAADAFYKLSNTKEQIQAYVFDVIRATIPTLELDKVFEQKTEIAKTVGERLEKVMSLYGYEILQALIVDVEPDDQVKSSMNEINAASRLREAAFEKAEGEKILQIKQAEGEAESKYLAGLGIARQRQAIVEGLKESVIGFSTSVPGASTKDVMDMILITQYFDAMKEISVSSNTSAVFIPHEPEDASSVQR

SEQ ID NO: 183 - protein, 266 aa, Medicago truncatula
MKEGFGKFEEVLQPGCHCMPWFLGKRIAGHLSLRLQQLDIKCETKTKDNVFVNVVASIQYRALADNANDAFYKLSNTRSQIQAYVFDEHMFQNSTWMILLSRKNEIAKAVEEELEKAMSAYGYEIVQTLIVDIEPDEHVKRAMNEINAAARMRVAAKEKAEAEKILQVKRAEGEAESKYLHGVGIARQRQAIVDGLRDSVIGFSVNVPGTSAKDVMDLVLVTQYFDTMKEIGAASKSSAVFIPHGPGAVRDVVSQIRDGLLQGSHE

SEQ ID NO: 184 - protein, 286 aa, Physcomitrella patens
MEGFGCLICVAQSTVGVIEKWGKFSGLAQPGLHCLNPFTGEWLAGRLSLRVQSLDVRCDTKTKDNVFVSVVCSIQYRVVRQNADDAFYELQNPKEQIQSYVFDVVRACVPRMILDDVFEQKDDIAKAVSEELEKVMGAYGYSIEQTLIVDIIPDSTVRRAMNEINAAQRMRMAAFDKAEAEKILQVKKAEGEAEAKYLNGRGIARQRQAITDGLRESVLQFSNNVPGTTSKDVMDLVLITQYFDTMKEIGAGSKNTTVFLPHGPGHVGDITQQIRDGMMQASASSD

SEQ ID NO: 185 - protein, 295 aa, Sorghum bicolor
MGAGSSSSIHSADLPKLLCCACVEQSTVAMEETCGRYDTVLQPGCHFMPWCVGRRVAGYLSLRVQQLDVRCETKSKDNVFVTVVASVQYRAIADKAYDAFYRLSNAREQIQSYVFDVIRASVPNMNLDQVFEQKNEVARAVEEELAKAMTMYGYEIVQTLIIDIEPDEVVKRAMNDINAAARLRVAAAERAEADKIQQVKRAEGEAESKYLAGVGVARQRQAIVEGLRRFVPDEKSVMDMVLATQYFDTIRDIGATSRAATVFIPHGPAAVHDMAAQVRDGVLQAAAYAPGAGTK

SEQ ID NO: 186 - protein, 292 aa, Oryza brachyantha
CAAEAAQALCCACVGQSTVAVGESWGRYDAVLGPGCHFVPWCVGRRVAGYLSLRVQQLDVRCETKTKDNVFVTVVASVQYRALADKAYDAFYRLTNARAQIQSYVFDVIRASVPNMNLDEVFGQKKEVAQAVEEELAKAMTMYGYEIVQTLIVDVVPDEVVKRAMNDINAAARLRVAAAERAEAEKIQQVKRAEGEAEAKYLAGVGVARQRQAIVEGLKRFVPNEKDVMDMVLVTQYFDTIRDIGATSRSSTVFIPHGPSAVRDMAAQVRDGLLQATATAAGGGGPAALKSL

SEQ ID NO: 187 - protein, 209 aa, Setaria italica
MEDKASDAYYKLSNPKAQIQSYVFDVIRASVPKLELDDAFEQKNEIAKAVEEELEKAMSAYGYEIVQTLIVDIEPDEKVKRAMNEINAAARLRVAANEKAEAEKIIQIKRAEGEAEAKYLSGLGIARQRQAIVDGLRDSVLGFSGNVPGTSAKDVMDLVLLTQYFDTMKEIGAASKSSAVFLPHGPGAVADIASQIRDGFLQASTQQAK

SEQ ID NO: 188 - protein, 290 aa, Amborella trichopoda
MGNTIGCICIDQASIGIVEKWGRFQRLAEPGFHLINPLAGEWIAGILSTRLSYLDVRVETKTKDNVFVQLLCSIQYRIVRQNADDAFYELQNPQEQIQAYVFDVVRAHVPRMTLDDLFEQKGDVGKAVLEELEKVMGAYGYNIEQILMVDIIPDPSVRKAMNEINAAQRLQLASVYKGEAEKVLQVKKAEGEAEAKFLGGVGVARQRQAITEGLRENVLDFSHGVPGTSAKDVMDLIMITQYFDTIRDLGNSSKNTTVFIPHGPGHVRDISDQIRNGLMEAASSSLENAN

SEQ ID NO: 189 - protein, 311 aa, Oryza sativa
MGGVTSSTSSSSPATAAGGSSYSAEAAQALCCACVGQSTVAVEEAWGRYDAVLGPGCHFVPWCVGRRVAGYLSLRVQQLDVRCETKTRDNVFVTVVASVQYRALADRAYDAFYCLTNAHAQIQSYVFDVIRASVPNMNLDEVFGQKKEVARAVEEELARAMTMYGYEIVQTLIVDIVPDEVVRRAMNDINAAARLRVAAAERAEADKIQQVKRAEGEAEAKYLAGVGVARQRQAIVEGLKRFVPNEKDVMDMVLVTQYFDTIRDIGATSRSSTVFIPHGPSAVRDMAAQVRDGLLQATAAAGGGAATLKAL

SEQ ID NO: 190 - protein, 291 aa, Eutrema salsugineum
MGNTFCIFGGCIDQASVGVVERWGRFEHIAEPGCHFFNPLAGQWLAGVLSTRINSLDVKIETKTKDNVFVQLVCSIQYRVVKTSADDAFYELQNPREQIQAYVFDVVRALVPMMTLDALFEQKGEVAKSVLEELEKVMGAYGYSIEHILMVDILPDPSVRKAMNEINAAQRLQLASVYKGEAEKILQVKRAEAEAESKYLGGVGVARQRQAITDGLRENIMNFSSKVEGTSAKEVMDLIMITQYFDTMRDLGNSSKNTTVFLPHGPGHVRDISDQIRNGLMEAASAHVSDA

SEQ ID NO: 191 - protein, 245 aa, Glycine max
MGQALGCYQVDQSNVAIKEHFGKFDDVLEPGCHCLPWCLGYQIAGSLSLRVQQLDVRCETKTKDNVFVTVVASVQYRAVSEKASDAFYRLTNTREQIQSYVFDVIRASVPKLELDSVFEQKNDIAKAVEEELEKAMSTYGFEIVQTLIVDIEPDVNVKRAMNEINAAARLRLAANEKAEAEKILQIKKAEGEAESKYLSGLGIARQRQAIVDGLRDSALAFSENVPGTTAKDVMDMVLVTHTLTP

SEQ ID NO: 192 - protein, 292 aa, Cicer arietinum
MGNTFCFFCGCVEQSSVGVVEQWGRFHRLAQPGFQFFNPFAGECLAGILSTRIASLDVKIETKTKDNVFVQLLCSIQYRVVKENADDAFYELQNPQEQIQAYVFDVARAIVPKMSLDDLFEQKGEVAKAVLDELEKVMGEYGYSIEHILMVDIIPDPSVRRAMNEINAAQRMLLASEFKGEAEKLLLVKKAEAEAESKYLGGVGVARQRQAITDGLRENILEFSNKVEGTSAKEVMDLIMITQYFDTIRDLGNSSKNTTIFIPHGPGHVRDIGEQIRNGMMEASCAQVTDIE

SEQ ID NO: 193 - protein, 292 aa, Arabidopsis thaliana
MGNTYCILGGCIEQASVGVVERWGRFEHIAEPGCHFFNPLAGQWLAGVLSTRIKSLDVKIETKTKDNVFVQLVCSIQYRVVKASADDAFYELQNPKEQIQAYVFDVVRALVPMMTLDALFEQKGEVAKSVLEELEKVMGAYGYSIEHILMVDIIPDPSVRKAMNEINAAQRLQLASVYKGEAEKILQVKRAEAEAEAKYLGGVGVARQRQAITDGLRENILNFSDKVEGTSAKEVMDLIMITQYFDTIRDLGNSSKNTTVFLPHGPGHVRDISDQIRNGMMEAAASTQVNDV

SEQ ID NO: 194 - protein, 290 aa, Citrus sinensis
MGNSFCMLCGCIDQASVGIVERWGRFEKLAEPGLHFFNPCAGQWLAGILSTRINSLDVRIETKTKDNVFVQLLCSIQYRIVRANADDAFYELQNPKEQIQAYVFDVVRALVPRMTLDELFEQKGEVAKAVLEELEKVMGAYGYSIEHILMVDIIPDPAVRKAMNEINAAQRLQLASVYKGEAEKILQVKRAEAEAEAKYLGGVGVARQRQAITDGLRENILNFSHKVEGASAKEVMDLIMITQYFDTIKDLGNSSNNTTVFLPHGPGHVRDISDQIRNGLMEASAAQVDG

SEQ ID NO: 195 - protein, 309 aa, Capsella rubella
IYKPGTWSSSSFFECRVEMGNAYCILGGCIDQASVGVVERWGRFEHIAEPGCHFFNPLAGQWLAGVLSTRINSLDVKIETKTKDNVFVQLVCSIQYRVVKASADDAFYELQNPREQIQAYVFDVVRALVPMMTLDALFEQKGEVAKSVLEELEKVMGAYGYSIEHILMVDILPDPSVRKAMNEINAAQRLQLASVYKGEAEKILQVKRAEAEAEAKYLGGVGVARQRQAITDGLRENILNFSDKVTGTSAKEVMDLIMITQYFDTIRDLGNSSKNTTVFLPHGPGHVRDISDQIRNGLMEAASAQVNDD